(19) United States
(12) Patent Application Publication
(10) Pub. No.: US 2024/0010681 A1
(43) Pub. Date: Jan. 11, 2024

(54) PEPTIDES AND METHODS OF USE

(71) Applicant: REALTA LIFE SCIENCES, INC., Norfolk, VA (US)

(72) Inventors: Neel K. KRISHNA, Norfolk, VA (US); Kenji CUNNION, Norfolk, VA (US); Ulrich THIENEL, Norfolk, VA (US)

(73) Assignee: REALTA LIFE SCIENCES, INC., Norfolk, VA (US)

(21) Appl. No.: 18/029,206

(22) PCT Filed: Sep. 27, 2021
(86) PCT No.: PCT/US2021/052174

§ 371 Date: Mar. 29, 2023

Related U.S. Application Data
(60) Provisional application No. 63/185,831, filed on May 7, 2021.
(60) Provisional application No. 63/085,556, filed on Sep. 30, 2020.

(51) Int. Cl. C07K 7/08; A61P 37/02; A61K 47/10

(52) U.S. Cl. CPC C07K 7/08; A61P 37/02; A61K 47/10

(57) ABSTRACT

The present invention provides peptides that are synthetic modifications of Polar Assonant (PA) peptide including C-terminal PEGylation. The invention further provides methods of using least one synthetic peptide for regulating the complement system and interacting with neutrophils to alter their binding and activity.

### CROSS-REFERENCE TO RELATED APPLICATIONS

This application is a U.S. National Phase of International Patent Application No. PCT/US2021/052174, filed on Sep. 27, 2021, which claims priority to U.S. Provisional Application No. 63/085,556, filed on Sep. 30, 2020, and 63/185,831, filed on May 7, 2021, the disclosures of which are herein incorporated by reference in their entirety.

### SEQUENCE LISTING

The instant application contains a Sequence Listing which has been submitted electronically in ASCII format and is hereby incorporated by reference in its entirety. Said ASCII copy, created on Sep. 1, 2023, is named 251110_000188_SL.txt and is 1293 bytes in size.

### BACKGROUND OF THE INVENTION

### 1. Field of the Invention

Embodiments of the present invention relates generally to synthetic peptides and uses thereof for therapy and diagnostics, and more specifically to a PEGylated form of the synthetic peptide.

### 2. Background

The Complement System

The complement system, an essential component of the innate immune system, plays a critical role as a defense mechanism against invading pathogens, primes adaptive immune responses, and helps remove immune complexes and apoptotic cells. Three different pathways comprise the complement system: the classical pathway, the lectin pathway and alternative pathway. C1q and mannose-binding lectin (MBL) are the structurally related recognition molecules of the classical and lectin pathways, respectively. Whereas IgM or clustered IgG serve as the principal ligands for C1q, MBL recognizes polysaccharides such as mannan. Ligand binding by C1q and MBL results in the sequential activation of C4 and C2 to form the classical and lectin pathway C3-convertase, respectively. In contrast, alternative pathway activation does not require a recognition molecule, but can amplify C3 activation initiated by the classical or lectin pathways. Activation of any of these three pathways results in the formation of inflammatory mediators (C3a and C5a) and the membrane attack complex (MAC), which causes cellular lysis.

While the complement system plays a critical role in many protective immune functions, complement activation is a significant mediator of tissue damage in a wide range of autoimmune and inflammatory disease processes. (Ricklin and Lambris, “Complement-targeted therapeutics.” Nat Biotechnol 2007; 25(11):1265-75).

A need exists for complement regulators. On the one hand, the complement system is a vital host defense against pathogenic organisms. On the other hand, its unchecked activation can cause devastating host cell damage. Currently, despite the known morbidity and mortality associated with complement dysregulation in many disease processes, including autoimmune diseases such as systemic lupus erythematosus, myasthenia gravis, and multiple sclerosis, only two anti-complement therapies have recently been approved for use in humans: 1) eculizumab (Soliris™) and 2) ultomiris (Ravulizumab™), two humanized, long-acting monoclonal antibodies against C5 used in the treatment of paroxysmal nocturnal hemoglobinuria (PNH) and atypical hemolytic uremic syndrome (aHUS). PNH and aHUS are orphan diseases in which very few people are afflicted. Currently, no complement regulators are approved for the more common disease processes in which dysregulated complement activation plays a pivotal role. Dysregulated complement activation can play a role in both chronic disease indications and acute disease indications.

Developing peptides to inhibit classical, lectin and alternative pathways of the complement system is needed, as each of these three pathways have been demonstrated to contribute to numerous autoimmune and inflammatory disease processes. Specific blockade of classical and lectin pathways is particularly needed, as both of these pathways have been implicated in ischemia reperfusion-induced injury and other diseases in many animal models. Humans with alternative pathway deficiencies suffer severe bacterial infections. Thus, a functional alternative pathway is essential for immune surveillance against invading pathogens.

The PIC1 family of molecules comprise a collection of rationally designed peptides, based on a scrambled astroviral coat protein, that have several anti-inflammatory functional properties including inhibition of the classical pathway of complement, myeloperoxidase inhibition, neutrophil extracellular trap (NET) inhibition and antioxidant activity. The original compound is a 15 amino acid peptide sequence, IALILEPICCQERAA (SEQ ID NO: 1), with a C-terminal monodisperse 24-mer PEGylated moiety (IALILEPICCQERAA-dPEG24; PA-dPEG24; SEQ ID NO: 2) increasing its aqueous solubility. Additional characteristics of the PA-dPEG24 molecule are discussed herein.

The Complement System and Ocular Diseases

The complement system is active in maintaining immune homeostasis and protection of the eye from pathogens, which involves a complicated interplay between complement activation molecules and complement regulatory molecules to control potential infections. While the complement system is necessary for immune surveillance, excessive and dysregulated complement activation has been implicated in many intraocular inflammatory and corneal inflammatory diseases such as autoimmune and infectious uveitis, acute macular degeneration (AMD), dry eye disease (DED), infectious and non-infectious keratitis, corneal injury and repair, retinopathy of prematurity (ROP), ocular graft versus host disease (GvHD), diabetic retinopathy (Jha et al., 2007), macular edema following retinal vein occlusion (RVO) and diabetic macular edema (DME).

Neutrophils, Neutrophil Extracellular Traps, and Ocular Diseases

Recently it has been reported that neutrophils have been demonstrated to play a critical role in the pathogenesis of AMD as seen in a mouse model and ex vivo studies on cadaveric human eye from AMD patients (Ghosh et al., 2019). In these studies, elevated interferon lambda in both human and mouse eyes were identified, and this high expression of interferon lambda induced the transmigration of neutrophils from the venous circulation to the retina eventually leading to pathological damage to the eyes.

In addition to a generalized role of neutrophils in eye disease, neutrophil extracellular traps (NETs) have been demonstrated to specifically play a pathogenic role in various other eye disease, such as chronic inflammation of the cornea, DED, infectious keratitis, corneal injury, ocular GvHD, non-infectious uveitis (e.g., Behcet's disease) as well as infectious uveitis, diabetic retinopathy, and finally AMD (Estua-Acosta et al., 2019; Ghosh et al., 2019). Specifically, in the case of AMID, NETosis biomarkers, myeloperoxidase (MPO), neutrophil elastase and citrullinated histone H3 have been demonstrated to contribute to pathogenesis in a mouse model of AMD (Ghosh et al., 2019).

The Complement System and Acute Lung Injury (ALI) and Acute Respiratory Distress Syndrome (ARDS)

ALI is often a complication of severe trauma that can progress to ARDS resulting in significant morbidity and mortality [Mica J, Jor O, Holub M, Sklienka P, Bursa F, Burda M, et al. Past and Present ARDS Mortality Rates: A Systematic Review. Respir Care 2017; 62(1):113-122]. To date, there are no pharmacological interventions to prevent ALI with current standard of care being supportive in nature. ALI may result from a combination of the underlying clinical condition of the patient (e.g., inflammation, trauma, hypotension) with a secondary insult such as a blood transfusion (transfusion-related ALI (TRALI), resuscitation, radiation) [Cho M S, Modi P, Sharma S. Transfusion-related Acute Lung Injury. 2020; In: StatPearls. Treasure Island (FL): StatPearls Publishing; 2020 January; Kumar A K, Anjum F. Ventilator-Induced Lung Injury (VILI). 2020 Dec. 15. In: StatPearls. Treasure Island (FL): StatPearls Publishing; 2020 January; Arroyo-Hernández M, Maldonado F, Lozano-Ruiz F, Muñoz-Montano W, Nunez-Baez M, Arrieta O. Radiation-induced lung injury: current evidence. BMC Pulm Med 2021; 21(1):9.] or viral pneumonia (e.g., influenza, respiratory syncytial virus or coronavirus-related ALI) [Klomp M, Ghosh S, Mohammed S, Nadeem Khan M. From virus to inflammation, how influenza promotes lung damage. J Leukoc Biol 2020; September 8; Alvarez A E, Marson F A, Bertuzzo C S, Arns C W, Ribeiro J D. Epidemiological and genetic characteristics associated with the severity of acute viral bronchiolitis by respiratory syncytial virus. J Pediatr (Rio J)2013; 89(6):531-43; Lee C, Choi W J. Overview of COVID-19 inflammatory pathogenesis from the therapeutic perspective. Arch Pharm Res 2021; January 4:1-18]. While the secondary insult may differ, the rapidly progressive disease process leading to pulmonary failure is typically mediated by an exaggerated and overwhelming innate immunological or inflammatory response driven by excessive complement and neutrophil-mediated inflammatory responses. In addition to ALI, dysregulated neutrophil and complement activation are key mediators of acute exacerbations in chronic lung diseases such as COPD and steroid resistant neutrophilic asthma [Pandya P H, Wilkes D S. Complement system in lung disease. Am J Respir Cell Mol Biol 2014; 51:467-473; Khan M A, Nicolls M R, Surguladze B, Saadoun I. Complement components as potential therapeutic targets for asthma treatment. Respir Med 2014; 108:543-549].

In the case of TRALI, which represents one of the leading causes of transfusion-related mortality, this disease process is complex and not fully understood, however a ‘two-hit’ model is currently believed to most accurately exemplify the clinical situation with the first hit mediated by the underlying clinical condition of the patient and the second hit triggered by a component in the transfused unit [Silliman C C, Paterson A J, Dickey W O, Stroneck D F, Popovsky M A, Caldwell S A, et al. The association of biologically active lipids with the development of transfusion-related acute lung injury: a retrospective study. Transfusion 1997; 37(7):719-26; Silliman C C, McLaughlin N J. Transfusion-related acute lung injury. Blood Rev 2006; 20(3):139-59]. Various in vitro, in vivo and ex vivo studies have implicated neutrophils as a key player in the pathogenesis of TRALI through direct activation, formation of reactive oxygen species (ROS) and neutrophil extracellular trap (NET) formation resulting in acute lung injury (ALI) [Rebetz J, Semple J W, Kapur R. The Pathogenic Involvement of Neutrophils in Acute Respiratory Distress Syndrome and Transfusion-Related Acute Lung Injury. Transfus Med Hemother 2018; 45(5):290-298]. Additionally, it has previously been postulated that the complement system may play a role in TRALI through C3a and C5a interaction with neutrophils resulting in neutrophil activation as well as ROS and NET formation [Jongerius I, Porcelijn L, van Beek A E, Semple J W, van der Schoot C E, Vlaar A P J, et al. The Role of Complement in Transfusion-Related Acute Lung Injury. Transfus Med Rev 2019; 33(4):236-242].

### Asthma

Bronchial asthma is a chronic, heterogeneous, inflammatory disease mediated by distinct immunopathologic mechanisms that include eosinophilic, neutrophilic, mixed granulocytic and paucigranulocytic asthma. It is estimated that between 3.6-10% of patients with asthma have severe, uncontrolled disease that is refractory to corticosteroids and β2-agonists which represent the standard drugs used for the treatment of asthma [Syabbalo N (2020) Clinical Features and Management of Neutrophilic Asthma. J Pulm Med Respir Res 6: 036]. Neutrophilic asthma is the most common form of acute severe asthma seen in adults. Patients with neutrophilic asthma are characterized by frequent emergency department visits, hospitalization, and intubation with sudden-onset fatal asthma in approximately 23% of patients [Fahy J V, Kim K W, Liu J, Boushey H A (1995) Prominent neutrophil inflammation in sputum from subjects with asthma exacerbations. J Allergy Clin Immunol 95: 843-852; Sur S, Crotty T B, Kephart G M, Hyama B A, Colby T V, et al. (1993) Sudden-onset fatal asthma: A distinct entity with few eosinophils and relatively more neutrophils in the airway mucosa? Am Rev Respir Dis 148:713-719]. The inability to control steroid-refractory, neutrophilic asthma currently represents an unmet clinical need.

The pathophysiological role of neutrophils in severe asthma has been demonstrated in human ex vivo studies. Recently, NETs, extracellular DNA and other neutrophil-derived products have been considered as possible biomarkers and therapeutic targets for severe asthma [Lachowicz-Scroggins M E, Dunican E M, Charbit A R, Raymond W, Looney M R, Peters M C, et al. (2019) Extracellular DNA, Neutrophil Extracellular Traps, and Inflammasome Activation in Severe Asthma. Am J Respir Crit Care Med. 199(9):1076-1085; Varricchi G, Modestino L, Poto R, Cristinziano L, Gentile L, Postiglione L, et al. (2021) Neutrophil extracellular traps and neutrophil-derived mediators as possible biomarkers in bronchial asthma. Clin Exp Med. 2021 Aug. 3. doi: 10.1007/s10238-021-00750-8].

The inventors have found that the PIC1 peptide can modulate neutrophil activity and therefore assessed the efficacy of RLS-0071 in a rat model of neutrophilic asthma using ovalbumin (OVA) and lipopolysaccharide (LPS) allergens.

### Angiogenesis

Angiogenesis is the process through which new blood vessels form from pre-existing vessels and continues the growth of the vasculature by the processes of sprouting and splitting. Angiogenesis is a normal physiological process in growth and development and also plays a critical role in wound healing and in the formation of granulation tissue. However, it is also a critical factor in the growth of tumors and plays a pathogenic role in many ocular conditions such as acute macular degeneration (AMD), retinopathy of prematurity (ROP) and diabetic retinopathy leading to the use of angiogenesis inhibitors in the treatment of cancer and ophthalmological diseases, respectively. VEGF is a major player in the process of angiogenesis and many angiogenesis inhibitory drugs target VEGF. However, VEGF-independent angiogenesis also occurs in a variety of inflammatory disease states. Thus, the inventors wished to study the effects of the PIC1 peptides on VEGF.

There is a need in the art for peptide-based inhibitors of the different pathways of the complement system. There is also a need in the art for therapeutic peptides to treat ophthalmic diseases and/or conditions as well ALI and/or ARDS, asthma, and to modulate angiogenesis.

### BRIEF SUMMARY OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to synthetic peptides that are PEGylated and their use in methods of regulating the complement system and interacting with neutrophils to regulate their binding and other activities.

In one aspect, the present invention provides synthetic peptides that regulate the complement system and methods of using these peptides. Specifically, in some embodiments, the synthetic peptides can bind, regulate, and inactivate C1 and MBL, and therefore can efficiently inhibit classical and lectin pathway activation at its earliest point of the complement cascade while leaving the alternative pathway intact. These peptides are of therapeutic value for selectively regulating and inhibiting C1 and MBL activation without affecting the alternative pathway and can be used for treating diseases mediated by dysregulated activation of the classical and lectin pathways. In other embodiments, the peptides regulate classical pathway activation but not lectin pathway activation. The peptides are useful for various therapeutic indications.

In other embodiments, the synthetic peptides are capable of altering cytokine expression, including but not limited to cytokine expression in models of ALI and/or ARDS.

In other embodiments, the synthetic peptides are capable of inhibiting or altering neutrophil binding and/or adhesion.

In other embodiments, the synthetic peptides are capable of improving neutrophil survival.

In other embodiments, the synthetic peptides can bind cell surface receptors such as for example but not limited to integrin and intercellular adhesion molecules (ICAMs), in vivo.

In some embodiments, the invention is based on the identification and modification of peptides of 15 amino acids from Polar Assortant (PA) peptide (SEQ ID NO: 1), derivatives of the peptides, and methods of their use. The PA peptide is a scrambled peptide derived from human astrovirus protein, called CP1. The PA peptide is also known as PIC1 (Peptide Inhibitors of Complement C1), AstroFend, AF, or SEQ ID NO: 1. The PIC1 peptide was originally named as such because it was found to be associated with diseases mediated by the complement system. A PEGylated form of the PIC1 peptide, called PA-dPEG24 (SEQ ID NO: 2; RLS-0071), has 24 PEG units on the C terminus of the peptide and was shown to have improved solubility in aqueous solution. A sarcosine substituted form of the PIC1 peptide, called PA-I8Sar (SEQ ID NO: 3; RLS-0088), has a sarcosine substituted for the isoleucine at position 8 of the peptide.

In an aspect, the present invention provides a method of altering cytokine expression comprising administering to the subject in need thereof a composition comprising a therapeutically effective amount of a synthetic peptide comprising SEQ ID NO: 2 and/or 3.

In an aspect, the present invention provides a method of inhibiting or altering neutrophil binding and/or adhesion comprising administering to the subject in need thereof a composition comprising a therapeutically effective amount of a synthetic peptide comprising SEQ ID NO: 2 and/or 3.

In an aspect, the present invention provides a method of improving neutrophil survival comprising administering to the subject in need thereof a composition comprising a therapeutically effective amount of a synthetic peptide comprising SEQ ID NO: 2 and/or 3.

In an aspect, the present invention provides a method of inhibiting or altering neutrophil binding to cell surface receptors comprising administering to the subject in need thereof a composition comprising a therapeutically effective amount of a synthetic peptide comprising SEQ ID NO: 2 and/or 3.

In an aspect, the present invention provides a method of treating a disease or condition characterized by an altered expression of a cell surface receptor comprising administering a composition comprising a therapeutically effective amount of a synthetic peptide comprising SEQ ID NO: 2 and/or 3.

In an aspect, the present invention provides a method of treating and/or preventing ALI and ARDS comprising administering a composition comprising a therapeutically effective amount of a synthetic peptide comprising SEQ ID NO: 2 and/or 3.

In an aspect, the present invention provides a method of treating and/or preventing an ocular disease and/or condition characterized by dysregulated complement activation and/or neutrophil modulation comprising administering a composition comprising a therapeutically effective amount of a synthetic peptide comprising SEQ ID NO: 2. In some embodiments, the ocular disease or condition is characterized by complement inhibition and/or inhibition of myeloperoxidase activity or NETosis. In some embodiments, the ocular disease or condition is autoimmune and infectious uveitis, acute macular degeneration (AMD), dry eye disease (DED), infectious and non-infectious keratitis, corneal injury and repair, retinopathy of prematurity (ROP), ocular graft versus host disease (GvHD), diabetic retinopathy, macular edema following retinal vein occlusion (RVO) and diabetic macular edema (DME).

In an aspect, the present invention provides a method of treating asthma comprising administering a composition comprising a therapeutically effective amount of a synthetic peptide comprising SEQ ID NO: 2. In some embodiments, the asthma is severe asthma, steroid-refractory asthma, or neutrophilic asthma.

In an aspect, the present invention provides a method of modulating angiogenesis comprising administering a composition comprising a therapeutically effective amount of a synthetic peptide comprising SEQ ID NO: 2 and/or 3.

In an embodiment of any of the foregoing methods, the composition further comprises at least one pharmaceutically acceptable carrier, diluent, stabilizer, or excipient. In an embodiment of any of the foregoing methods, the therapeutically effective amount of SEQ ID NO: 2 and/or 3 is about 10 mg/kg to about 160 mg/kg. In an embodiment of any of the foregoing methods, the therapeutically effective amount of SEQ ID NO: 2 and/or 3 is about 20 mg/kg to about 160 mg/kg. In an embodiment of any of the foregoing methods, the therapeutically effective amount of SEQ ID NO: 2 and/or 3 is about 40 mg/kg to about 160 mg/kg. In an embodiment of any of the foregoing methods, the therapeutically effective amount of SEQ ID NO: 2 and/or 3 is administered in at least one dose, the first dose comprising about 10 mg/kg to about 160 mg/kg SEQ ID NO: 2 and/or 3. In an embodiment of any of the foregoing methods, a second dose comprising a therapeutically effective amount of SEQ ID NO: 2 and/or 3 is administered, the second dose comprising about 40 mg/kg to about 60 mg/kg SEQ ID NO: 2 and/or 3. In an embodiment of any of the foregoing methods, the therapeutically effective amount of SEQ ID NO: 2 and/or 3 is administered in two doses, the first dose comprising about 10 mg/kg to about 160 mg/kg SEQ ID NO: 2 and/or 3 and the second dose comprising about 40 mg/kg to about 60 mg/kg SEQ ID NO: 2 and/or 3. In some embodiments, the second dose is administered 30 seconds to 3 hours after the first dose is administered. In an embodiment of any of the foregoing methods, the composition is formulated for ophthalmic administration. In an embodiment, the composition further comprises an ophthalmically acceptable carrier and/or excipient. In an embodiment of any of the foregoing methods, the ophthalmic administration comprises topical administration, periocular injection, subconjunctival injection, intra-aqueous injection, intraocular injection, intravitreal injection, or introduction of an intracorneal or intraocular implant. In an embodiment of any of the foregoing, the composition is formulated for nasal administration. In an embodiment of any of the foregoing, the nasal administration comprises inhalation, insufflation, or nebulization. In an embodiment of any of the foregoing, the nasal composition is in the form of a spray, solution, gel, cream, lotion, aerosol or solution for a nebulizer, or as a microfine powder for insufflation.

In an embodiment of any of the foregoing, the cell surface receptor comprises an integrin or an intercellular adhesion molecule (ICAM). In an embodiment of any of the foregoing, the ICAM comprises ICAM-1, ICAM-3, ICAM-4, and/or ICAM-5. In an embodiment of any of the foregoing, the disease or condition is characterized by an increase in at least one of ICAM-1, ICAM-3, ICAM-4, and/or ICAM-5.

These and other objects, features and advantages of the present invention will become more apparent upon reading the following specification in conjunction with the accompanying description, claims and drawings.

### BRIEF DESCRIPTION OF THE DRAWINGS

The accompanying Figures, which are incorporated in and constitute a part of this specification, illustrate several aspects described below.

FIG. 1 shows that intravenous (IV) administration of PA-dPEG24 (also referred to herein as RLS-0071) delivered before or after incompatible erythrocyte transfusion reduces levels of IFNgamma, IL-6, IL-2, IL-10, TNFalpha, MCP-1, RANTES, MIPlalpha, IL-1beta, MIP-2. Cytokine levels from terminal blood draws obtained from sham animals and animals receiving LPS alone, LPS+30% transfusion and LPS+30% transfusion and single doses of 10 or 160 mg/kg RLS-0071 administered before (prophylactic) or single doses of 40 and 160 mg/kg RLS-0071 administered after (rescue) transfusion were determined by xMAP bead-based immunoassay. Data are means and standard error of the mean. * denotes P<0.05, ** denotes P<0.01 compared to LPS+30% transfusion.

FIG. 2 shows that intravenous (IV) administration of RLS-0071 delivered before or after incompatible erythrocyte transfusion reduces levels of IL-5, IL-18, IL-1alpha, IL-13, IL-17, IL-12, and IP-10. Cytokine levels from terminal blood draws obtained from sham animals and animals receiving LPS alone, LPS+30% transfusion and LPS+30% transfusion and single doses of 10 or 160 mg/kg RLS-0071 administered before (prophylactic) or single doses of 40 and 160 mg/kg RLS-0071 administered after (rescue) transfusion were determined by xMAP bead-based immunoassay. Data are means and standard error of the mean. * denotes P<0.05, ** denotes P<0.01 compared to LPS+30% transfusion.

FIG. 3 shows that intravenous (IV) administration of RLS-0071 delivered before or after incompatible erythrocyte transfusion does not significantly affect levels of the anti-inflammatory cytokine IL-4. IL-4 from terminal blood draws obtained from sham animals and animals receiving LPS alone, LPS+30% transfusion and LPS+30% transfusion and single doses of 10 or 160 mg/kg RLS-0071 administered before (prophylactic) or single doses of 40 and 160 mg/kg RLS-0071 administered after (rescue) transfusion were determined by xMAP bead-based immunoassay. Data are means and standard error of the mean.

FIG. 4 shows the effects of intravenous (IV) administration of RLS-0071 delivered before or after incompatible erythrocyte transfusion on levels of EGF, LIX, VEGF, Leptin, GRO, Fractalkine, GM-CSF, Eotaxin and G-CSF. Cytokine and growth factor levels from terminal blood draws obtained from sham animals and animals receiving LPS alone, LPS+30% transfusion and LPS+30% transfusion and single doses of 10 or 160 mg/kg RLS-0071 administered before (prophylactic) or single doses of 40 and 160 mg/kg RLS-0071 administered after (rescue) transfusion were determined by xMAP bead-based immunoassay. Data are means and standard error of the mean. * denotes P<0.05, ** denotes P<0.01 compared to LPS+30% transfusion.

FIG. 5A-B shows staining of liver (5A) and kidney (5B) tissues for RLS-0071 from rats receiving intravenous (IV) administration of 400 mg/kg PA-dPEG24 compared to untreated animals. Liver (5A) and kidney (5B) tissue sections were stained for RLS-0071 and visualized by microscopy at 20× and 40× magnification. Brown staining indicates presence of RLS-0071 in the tissues. Red arrows in the liver sections denote punctate RLS-0071 staining.

FIG. 6 shows immunofluorescence staining for RLS-0071 demonstrating that the peptide binds to human neutrophils. Human neutrophils were adhered on glass slides, fixed with paraformaldehyde, and then incubated in the presence or absence of RLS-0071. The slides were then stained with antibody to RLS-0071 (Chicken Anti-PIC1) followed by a labeled secondary antibody (Anti-Chicken, Alexa Fluor 488) and counterstained with DAPI. Cells were subsequently visualized by microscopy.

FIG. 7 shows that RLS-0071 inhibits human neutrophil adhesion to glass slides. Images show human neutrophils adhered to the surface of glass slides in the presence of increasing concentrations of RLS-0071. Neutrophils were stained with DAPI and imaged with fluorescent microscopy. Representative images are shown. The graph in the bottom right panel shows the numbers of neutrophils adhered to a glass slide after incubation with increasing concentrations of RLS-0071 followed by washing with PBS before placement in on glass slide and incubation for 2.5 hours. His denotes cells treated with histidine buffer only (pH 6.5). Data are means of n=4 SEM.

FIG. 8 shows that RLS-0071 inhibits human neutrophil adhesion to glass slides with and without fibrinogen treatment. Images show human neutrophils adhered to the surface of fibrinogen coated glass slides in the presence of increasing concentrations of PA-DPEG24. Neutrophils were stained with DAPI and imaged with fluorescent microscopy. Representative images are shown. The graph in the bottom right panel shows the numbers of neutrophils after incubation with increasing concentrations of RLS-0071 followed by washing with PBS before placement on fibrinogen-coated glass or untreated glass slides and incubation for 2.5 hours.

FIG. 9 shows that RLS-0071 increases human neutrophil viability as measured by the CCK8 assay, which measures cellular respiration as an indication of viability (number of living cells). RLS-0071 dose-dependently increases human neutrophil viability in the CCK8 assay. Cells in PBS or RPMI were incubated with increasing amounts of RLS-0071. “Fresh” denotes unmanipulated cells that were plated with CCK8 for 2 hours at 37° C. immediately after the purification process was complete.

FIG. 10 shows that RLS-0071 can bind to both neutrophil receptor LFA-1 and epithelial cell receptor ICAM-1. RLS-0071 selectively binds purified endothelial and neutrophil cell receptors. Plates were coated with the purified neutrophil receptors LFA-1 and MAC-1 and endothelial cell receptors ICAM-1 and ICAM-2 and then incubated with increasing amounts of RLS-0071 in buffer. Plates were washed, and then incubated with rabbit anti-RLS0071 antisera, washed, and then incubated with anti-rabbit HRP. Plates were washed again and developed. Absorbance was read at 450 nm. PIC1=RLS-0071. C1q was used as a positive control for RLS-0071 binding.

FIG. 11 shows that RLS-0071 can bind to epithelial cell receptors ICAM-1, ICAM-3, ICAM-4, and ICAM-5. Plates were coated with the purified neutrophil receptors ICAM-1, ICAM-2, ICAM-3, ICAM-4, and ICAM-5. and then incubated with increasing amounts of RLS-0071 in buffer. Plates were washed, and then incubated with rabbit anti-RLS0071 antisera, washed, and then incubated with anti-rabbit HRP. Plates were washed again and developed. Absorbance was read at 450 nm. C1q was used as a positive control and ICAM-2 as a negative control for RLS-0071 binding.

FIG. 12 shows that RLS-0071 can bind to neutrophil receptor LFA-1 or endothelial cell ICAM-1 in plasma. Plates were coated with the purified receptors and then incubated with increasing amounts of RLS-0071 in human plasma. Plates were washed, and then incubated with affinity purified rabbit anti-RLS0071 antisera, washed, and then incubated with anti-rabbit HRP. Plates were washed again and developed. Absorbance was read at 450 nm. PIC1=RLS-0071. C1q and MPO (myeloperoxidase) were used as a positive control for RLS-0071 binding.

FIG. 13 shows radiochromatograms of time point pooled plasma from male Sprague-Dawley rats following a single IV dose of [14C]-PIC1-RLS-0071 at 20 mg/kg.

FIG. 14 shows radiochromatograms of time point pooled plasma from male Sprague-Dawley Rats following a single IV dose of [14C]-PIC1-RLS-0071 at 200 mg/kg.

FIG. 15 shows that RLS-0071 does not interfere with binding of C1q-immune complex binding to receptors on human monocytes. Human monocytes were purified and allowed to adhere to a microtiter plate. Heat-aggregated human immune complexes were allowed to bind C1q in the presence of increasing amount of RLS-0071. These complexes were then allowed to bind the monocytes and then washed and bound C1q/immune complexes detected by primary antibody followed by secondary antibody-HRP and developed with TMB. Absorbance was read at 450 nm. N=3. Bars indicate standard error of the mean (SEM).

FIGS. 16A-16C show that RLS-0071 reduces levels of inflammatory cytokines in the blood. Cytokine levels IL-1a, IFN-g, IL-1b, IL-6 (16A); IL-17, IL-18, TNFa and RANTES (16B); IL-4, IL-10, and VEGF (16C) from terminal blood draws were determined by xMAP bead based immunoassay for the following experimental groups: sham, first-hit only, 2-hit, 2-hit+10 mg/kg prophylactic dose RLS-0071, 2-hit+160 mg/kg prophylactic dose RLS-0071 as well as 2-hit+40 mg/kg rescue dose RLS-0071 and 2-hit+160 mg/kg rescue dose RLS-0071. For sake of clarity only rescue dosing data is shown. Data are means and standard error of the mean. * denotes P<0.05 compared to animals receiving the 2-hit insult.

FIGS. 17A-17C show that RLS-0071 reduces levels of inflammatory chemokines in the blood. Chemokine levels (17A) MCP-1, (17B) MIP-1a and (17C) MIP-2 from terminal blood draws were determined by xMAP bead-based immunoassay for the following experimental groups: sham, 1st-hit only, 2-hit, 2-hit+10 mg/kg prophylactic dose RLS-0071, 2-hit+160 mg/kg prophylactic dose RLS-0071 as well as 2-hit+40 mg/kg rescue dose RLS-0071 and 2-hit+160 mg/kg rescue dose RLS-0071. For sake of clarity only rescue dosing data is shown. Data are means and standard error of the mean. * denotes P<0.05 compared to animals receiving the 2-hit insult.

FIG. 18A-18K show that prophylactic or rescue dosing of RLS-0071 reduces acute lung injury. Representative histology (H&E stain) of rat lungs. (18A) sham control, (18B) first hit only, (18C) 2-hit, (18D) 2-hit+10 mg/kg prophylactic dose RLS-0071, (18E) 2-hit+40 mg/kg prophylactic dose RLS-0071, (18F) 2-hit+160 mg/kg prophylactic dose RLS-0071, (18G) 2-hit+40 mg/kg rescue dose RLS-0071 at 0.5 min, (18H) 2-hit+40 mg/kg rescue dose RLS-0071 at 60 min, (18I) 2-hit+40 mg/kg rescue dose RLS-0071 at 90 min, (18J) 2-hit+40 mg/kg rescue dose RLS-0071 at 120 min and (18K) 2-hit+40 mg/kg rescue dose RLS-0071 at 180 min. Bar represents 100 μm. Tissues were observed with a microscope (BX50, Olympus) at a magnification of 20× at room temperature. Images were acquired with a digital camera (DP70, Olympus).

FIG. 19 shows that prophylactic or rescue dosing of RLS-0071 reduces neutrophil-mediated lung injury. H&E-stained lung tissue images were converted to black and white and quantified by ImageJ analysis. The ratio of black to white pixels was calculated and used as a measure of lung injury (Y axis). Sham control animals (n=3), first hit only (n=2), 2-hit (n=3), 2-hit+10 mg/kg prophylactic dose RLS-0071 (n=4), 2-hit+40 mg/kg prophylactic dose RLS-0071 (n=6), 2-hit+160 mg/kg prophylactic dose RLS-0071 (n=9), 2-hit+40 mg/kg rescue dose RLS-0071 at 0.5 min (n=4), 2-hit+40 mg/kg rescue dose RLS-0071 at 60 min (n=3), 2-hit+40 mg/kg rescue dose RLS-0071 at 90 min (n=5), 2-hit+40 mg/kg rescue dose RLS-0071 at 120 min (n=3) and 2-hit+40 mg/kg rescue dose RLS-0071 at 180 min (n=3). Ten images or more were quantified per slide for each animal. Data are means and standard error of the means. Statistical analysis was performed using a Generalized Linear Model. * denotes p=0.002 and ** denotes p<0.001 compared to 2-hit animals.

FIG. 20 shows that RLS-0071 inhibits complement activation. Plasma was isolated from sham animals (n=3) and the following groups prior to first-hit (0 minutes) and at 5 minutes and 1 hour: first hit only (n=3), 2-hit (n=3), 2-hit+10 mg/kg prophylactic dose RLS-0071 (n=8), 2-hit+40 mg/kg prophylactic dose RLS-0071 (n=4), 2-hit+160 mg/kg prophylactic dose RLS-0071 (n=5), 2-hit+40 mg/kg rescue dose RLS-0071 at 0.5 min (n=5), 2-hit+40 mg/kg rescue dose RLS-0071 at 60 min (n=3), 2-hit+40 mg/kg rescue dose RLS-0071 at 90 min (n=5), 2-hit+40 mg/kg rescue dose RLS-0071 at 120 min (n=3) and 2-hit+40 mg/kg rescue dose RLS-0071 at 180 min (n=3). C5a was then measured in each sample by ELISA and absorbance was read at 450 nm. Two replicates for each animal were measured for every time point. Data are means and standard error of the mean. Statistical analysis was performed using were conducted using bootstrap approach or Welch's ANOVA. * denotes p=0.010, ** denotes p=0.004, *** denotes p=0.002, and **** denotes p≤0.001 compared to 2-hit animals.

FIG. 21 shows that RLS-0071 reduces free DNA levels in the blood. Plasma was isolated from sham animals (n=3) and the following groups at 4 hours after start of the experiments: first hit only (n=3), 2-hit (n=3), 2-hit+10 mg/kg prophylactic dose RLS-0071 (n=9), 2-hit+40 mg/kg prophylactic dose RLS-0071 (n=4), 2-hit+160 mg/kg prophylactic dose RLS-0071 (n=5), 2-hit+40 mg/kg rescue dose RLS-0071 at 0.5 min (n=4), 2-hit+40 mg/kg rescue dose RLS-0071 at 60 min (n=3), 2-hit+40 mg/kg rescue dose RLS-0071 at 90 min (n=5), 2-hit+40 mg/kg rescue dose RLS-0071 at 120 min (n=3) and 2-hit+40 mg/kg rescue dose RLS-0071 at 180 min (n=3). Plasma samples were incubated with PicoGreen. Fluorescence was read at an excitation wavelength of 485 nm and an emission wavelength of 520 nm in a microplate reader. All free DNA measurements for each animal were done in triplicate. Data are means and standard error of the mean. Statistical analysis was performed using bootstrap approach or Welch's ANOVA. * denotes p=0.026, ** denotes p=0.039 and *** denotes p=0.005 compared to 2-hit animals.

FIGS. 22A-22D show that RLS-0071 delivered via intravitreal (IVT) injection had a longer half-life than intravenous (IV) dosed RLS-0071. (22A) and (22B): rats dosed IVT with 160 mg/ml RLS-0071 were euthanized at the indicated time points and vitreous humor isolated. (22C) and (22D): rats dosed IV with 200 mg/ml RLS-0071 had blood drawn at the indicated time points and plasma isolated. The vitreous and plasma samples were then analyzed in a sandwich ELISA to detect levels of RLS-0071. Panels 22B and 22D are identical to Panels 22A and 22C, respectively, with the Y axis scaled to emphasize peptide levels at later time points.

FIG. 23 shows that RLS-0071 delivered via IVT stained retinal tissue 1 hour post administration. Rats were injected IVT with saline or 160 mg/kg RLS-0071. Animals were euthanized 5 minutes after saline infusion or 1 hour after RLS-0071 infusion and eyes processed for histology and staining with an antibody to RLS-0071 followed by detection by DAB staining. Images were observed by microscopy at a magnification of 4× (top panels) and 20× (bottom panels) five minutes after IVT (left panels) and one hour after IVT (right panels).

FIG. 24 shows C5a generation measured in the plasma of a two-hit rat model of acute lung injury.

FIG. 25 shows that incompatible erythrocytes transfused as the second hit in the 2-hit ALI model activated the classical complement pathway causing hemolysis releasing free hemoglobin into the blood measured in the plasma. Saline treated animals are represented in the middle columns and RLS-0071 animals are represented in the right-hand columns. Sham animals, in the left-hand columns, were not transfused.

FIG. 26 shows the results of a CH50 assay on plasma obtained from the 2-hit ALI animals. Saline treated animals are shown in right-hand columns and RLS-0071 animals are shown in the left-hand columns.

FIG. 27 shows the experimental design for testing the effects of RLS-0071 on severe asthma.

FIG. 28 shows that RLS-0071 reduces neutrophil levels in bronchoalveolar lavage fluid (BALF) of asthma rats. Upper Panel: representative BALF images are shown for each experimental group: sham control, animals that received intraperitoneal ovalbumin (OVA)/lipopolysaccharide (LPS) protocol (asthma, day 24), asthma animals that received prophylactic dose of 160 mg/kg RLS-0071 on Days 21, 22, 23 and animals that received a rescue dose of 160 mg/kg RLS-0071 on Days 22 and 23. All animals were sacrificed at Day 24 and BALF collected. BALF was observed by microscope (BX50, Olympus) at a magnification of 40× at room temperature. Images were acquired with a digital camera (DP70, Olympus). Lower panel: quantification of leucocytes by two independent readers. Cell counts are expressed as percent of total. Data are means and standard error of the mean. * denotes P<0.03 compared to asthma animals.

FIG. 29 shows that RLS-0071 reduces protein levels in BALF of asthma rats. The following experimental groups were evaluated: sham animals (unstimulated), animals receiving OVA/LPS protocol (asthma), asthma animals receiving prophylactic dose of 160 mg/kg RLS-0071 on Days 21, 22, 23 and animals receiving rescue dose of 160 mg/kg RLS-0071 on Days 22 and 23. Groups of asthma rats were sacrificed at Days 20-24 and asthma rats that received RLS-0071 were sacrificed on Day 24, BALF fluid collected, and total protein levels determined by BCA protein assay. Data are means and standard error of the mean.

FIG. 30 shows that RLS-0071 reduces free MPO levels in BALF of asthma rats. The following experimental groups were evaluated: sham animals (unstimulated), animals receiving OVA/LPS protocol (asthma), asthma animals receiving prophylactic dose of 160 mg/kg RLS-0071 on Days 21, 22, 23 and animals receiving rescue dose of 160 mg/kg RLS-0071 on Days 22 and 23. Groups of asthma rats were sacrificed at Days 20-24 and asthma rats that received RLS-0071 were sacrificed on Day 24, BALF fluid collected and MPO levels determined by colorimetric assay. Data are means and standard error of the mean. * denotes P=0.05 compared to asthma animals (Day 24).

FIG. 31 shows that RLS-0071 reduces free DNA levels in BALF of asthma rats. The following experimental groups were evaluated: sham animals (unstimulated), animals receiving OVA/LPS protocol (asthma), asthma animals receiving prophylactic dose of 160 mg/kg RLS-0071 on Days 21, 22, 23 and animals receiving rescue dose of 160 mg/kg RLS-0071 on Days 22 and 23. Groups of asthma rats were sacrificed at Days 20-24 and asthma rats that received RLS-0071 were sacrificed on Day 24, BALF fluid collected, and free DNA levels determined by PicoGreen assay. Data are means and standard error of the mean.

FIG. 32 shows that RLS-0071 binds to human VEGF in a dose-dependent manner. VEGF was coated onto a microtiter plate and incubated with RLS-0071 at increasing concentration which were subsequently detected with an antibody to the peptide, followed by secondary antibody-HRP conjugate. The signal generated from the HRP conjugate was then read in a plate reader at an OD of 450 nm. C1q was used as a positive control for binding.

FIG. 33 shows that RLS-0088 has low levels of binding to human VEGF. VEGF was coated onto a microtiter plate and incubated with 1 mg/ml RLS-0071 (positive control) or RLS-0088. Peptides were subsequently detected with an antibody to the peptide, followed by secondary antibody-RP conjugate. The signal generated from the HRP conjugate was then read in a plate reader at an OD of 450 nm.

FIG. 34 shows that RLS-0071 and RLS-0088 inhibits VEGF binding to VEGFR-2 and cell signaling. To assess the ability of RLS-0071 and RLS-0088 to inhibit VEGF signaling, Promega's VEGF Bioassay was utilized. This bioluminescent cell-based assay measures VEGF binding to VEGFR-2 on reporter cells using luciferase as a readout. The bioluminescent signal is detected and quantified using Bio-Glo™ Luciferase Assay System and a standard luminometer. Increasing concentrations of VEGF led to a dose-dependent increase in luminescence (positive control, diamonds). Cells were incubated with increasing concentrations of the peptides followed by stimulation of the cells with human VEGF (black line showing level of VEGF stimulation alone as a reference). Both RLS-0071 and RLS-0088 inhibited VEGF-mediated signaling in a dose-dependent fashion (squares and triangles, respectively).

FIG. 35. RLS-0071 and RLS-0088 inhibits angiogenesis in a human umbilical vascular endothelial cell (HUVEC) 3-dimensional culture system. Purified HUVECs stained with a CellTrace dye, preincubated with RLS-0071 and RLS-0088 and then added to extracellular matrix containing LPS to stimulate angiogenesis. Cells were then incubated at 37° C. overnight and observed for angiogenesis (nascent tube formation and sprouting) by visualization on an inverted microscope.

FIG. 36. RLS-0071 inhibits angiogenesis in a HUVEC basement membrane-mediated culture system. Purified HUVECs were preincubated with RLS-0071 at increasing concentrations for 30 min. Cells were applied to a layer of basement membrane matrix containing LPS to stimulate angiogenesis and cultured for 18 hours at 37° C. Angiogenesis (nascent tube formation and sprouting) was observed by light microscopy.

### DETAILED DESCRIPTION OF THE INVENTION

As specified in the Background Section, there is a great need in the art to identify technologies for peptide-based inhibitors of the different pathways of the complement system and use this understanding to develop novel therapeutic peptides. The present invention satisfies this and other needs. Embodiments of the present invention relate generally to synthetic peptides and more specifically to PEGylated forms of the synthetic peptides.

To facilitate an understanding of the principles and features of the various embodiments of the invention, various illustrative embodiments are explained below. Although exemplary embodiments of the invention are explained in detail, it is to be understood that other embodiments are contemplated. Accordingly, it is not intended that the invention is limited in its scope to the details of construction and arrangement of components set forth in the following description or examples. The invention is capable of other embodiments and of being practiced or carried out in various ways. Also, in describing the exemplary embodiments, specific terminology will be resorted to for the sake of clarity.

It must also be noted that, as used in the specification and the appended claims, the singular forms “a,” “an” and “the” include plural references unless the context clearly dictates otherwise. For example, reference to a component is intended also to include composition of a plurality of components. References to a composition containing “a” constituent is intended to include other constituents in addition to the one named. In other words, the terms “a,” “an,” and “the” do not denote a limitation of quantity, but rather denote the presence of “at least one” of the referenced item.

As used herein, the term “and/or” may mean “and,” it may mean “or,” it may mean “exclusive-or,” it may mean “one,” it may mean “some, but not all,” it may mean “neither,” and/or it may mean “both.” The term “or” is intended to mean an inclusive “or.”

Also, in describing the exemplary embodiments, terminology will be resorted to for the sake of clarity. It is intended that each term contemplates its broadest meaning as understood by those skilled in the art and includes all technical equivalents which operate in a similar manner to accomplish a similar purpose. It is to be understood that embodiments of the disclosed technology may be practiced without these specific details. In other instances, well-known methods, structures, and techniques have not been shown in detail in order not to obscure an understanding of this description. References to “one embodiment,” “an embodiment,” “example embodiment,” “some embodiments,” “certain embodiments,” “various embodiments,” etc., indicate that the embodiment(s) of the disclosed technology so described may include a particular feature, structure, or characteristic, but not every embodiment necessarily includes the particular feature, structure, or characteristic. Further, repeated use of the phrase “in one embodiment” does not necessarily refer to the same embodiment, although it may.

As used herein, the term “about” should be construed to refer to both of the numbers specified as the endpoint (s) of any range. Any reference to a range should be considered as providing support for any subset within that range. Ranges may be expressed herein as from “about” or “approximately” or “substantially” one particular value and/or to “about” or “approximately” or “substantially” another particular value. When such a range is expressed, other exemplary embodiments include from the one particular value and/or to the other particular value. Further, the term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to +5%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Similarly, as used herein, “substantially free” of something, or “substantially pure”, and like characterizations, can include both being “at least substantially free” of something, or “at least substantially pure”, and being “completely free” of something, or “completely pure”.

By “comprising” or “containing” or “including” is meant that at least the named compound, element, particle, or method step is present in the composition or article or method, but does not exclude the presence of other compounds, materials, particles, method steps, even if the other such compounds, material, particles, method steps have the same function as what is named.

Throughout this description, various components may be identified having specific values or parameters, however, these items are provided as exemplary embodiments. Indeed, the exemplary embodiments do not limit the various aspects and concepts of the present invention as many comparable parameters, sizes, ranges, and/or values may be implemented. The terms “first,” “second,” and the like, “primary,” “secondary,” and the like, do not denote any order, quantity, or importance, but rather are used to distinguish one element from another.

It is noted that terms like “specifically,” “preferably,” “typically,” “generally,” and “often” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention. It is also noted that terms like “substantially” and “about” are utilized herein to represent the inherent degree of uncertainty that may be attributed to any quantitative comparison, value, measurement, or other representation.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “50 mm” is intended to mean “about 50 mm.”

It is also to be understood that the mention of one or more method steps does not preclude the presence of additional method steps or intervening method steps between those steps expressly identified. Similarly, it is also to be understood that the mention of one or more components in a composition does not preclude the presence of additional components than those expressly identified.

The materials described hereinafter as making up the various elements of the present invention are intended to be illustrative and not restrictive. Many suitable materials that would perform the same or a similar function as the materials described herein are intended to be embraced within the scope of the invention. Such other materials not described herein can include, but are not limited to, materials that are developed after the time of the development of the invention, for example. Any dimensions listed in the various drawings are for illustrative purposes only and are not intended to be limiting. Other dimensions and proportions are contemplated and intended to be included within the scope of the invention.

As used herein, the term “subject” or “patient” refers to mammals and includes, without limitation, human and veterinary animals. In a preferred embodiment, the subject is human.

As used herein, the term “combination” of a synthetic peptide according to the claimed invention and at least a second pharmaceutically active ingredient means at least two, but any desired combination of compounds can be delivered simultaneously or sequentially (e.g., within a 24-hour period). It is contemplated that when used to treat various diseases, the compositions and methods of the present invention can be utilized with other therapeutic methods/agents suitable for the same or similar diseases. Such other therapeutic methods/agents can be co-administered (simultaneously or sequentially) to generate additive or synergistic effects. Suitable therapeutically effective dosages for each agent may be lowered due to the additive action or synergy.

A “disease” is a state of health of a subject wherein the subject cannot maintain homeostasis, and wherein if the disease is not ameliorated then the subject's health continues to deteriorate. In contrast, a “disorder” in a subject is a state of health in which the subject is able to maintain homeostasis, but in which the subject's state of health is less favorable than it would be in the absence of the disorder. Left untreated, a disorder does not necessarily cause a further decrease in the subject's state of health.

The terms “treat” or “treatment” of a state, disorder or condition include: (1) preventing or delaying the appearance of at least one clinical or sub-clinical symptom of the state, disorder or condition developing in a subject that may be afflicted with or predisposed to the state, disorder or condition but does not yet experience or display clinical or subclinical symptoms of the state, disorder or condition; or (2) inhibiting the state, disorder or condition, i.e., arresting, reducing or delaying the development of the disease or a relapse thereof (in case of maintenance treatment) or at least one clinical or sub-clinical symptom thereof, or (3) relieving the disease, i.e., causing regression of the state, disorder or condition or at least one of its clinical or sub-clinical symptoms. The benefit to a subject to be treated is either statistically significant or at least perceptible to the patient or to the physician.

The term “therapeutic” as used herein means a treatment and/or prophylaxis. A therapeutic effect is obtained by suppression, diminution, remission, or eradication of a disease state.

As used herein the term “therapeutically effective” applied to dose or amount refers to that quantity of a compound or pharmaceutical composition that when administered to a subject for treating (e.g., preventing or ameliorating) a state, disorder or condition, is sufficient to effect such treatment. The “therapeutically effective amount” will vary depending on the compound or bacteria or analogues administered as well as the disease and its severity and the age, weight, physical condition, and responsiveness of the mammal to be treated.

The phrase “pharmaceutically acceptable”, as used in connection with compositions of the invention, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans.

The terms “pharmaceutical carrier” or “pharmaceutically acceptable carrier” refer to a diluent, adjuvant, excipient, or vehicle with which the compound is administered. Such pharmaceutical carriers can be sterile liquids, such as water and oils, including those of petroleum, animal, vegetable, or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Alternatively, the pharmaceutical carrier can be a solid dosage form carrier, including but not limited to one or more of a binder (for compressed pills), a glidant, an encapsulating agent, a flavorant, and a colorant. Suitable pharmaceutical carriers are described in “Remington's Pharmaceutical Sciences” by E. W. Martin.

The term “analog” or “functional analog” refers to a related modified form of a polypeptide, wherein at least one amino acid substitution, deletion, or addition has been made such that said analog retains substantially the same biological activity as the unmodified form, in vivo and/or in vitro.

The terms “sequence identity” and “percent identity” are used interchangeably herein. For the purpose of this invention, it is defined here that in order to determine the percent identity of two amino acid sequences or two nucleic acid sequences, the sequences are aligned for optimal comparison purposes (e.g., gaps can be introduced in the sequence of a first amino acid or nucleic acid for optimal alignment with a second amino or nucleic acid sequence). The amino acid or nucleotide residues at corresponding amino acid or nucleotide positions are then compared. When a position in the first sequence is occupied by the same amino acid or nucleotide residue as the corresponding position in the second sequence, then the molecules are identical at that position. The percent identity between the two sequences is a function of the number of identical positions shared by the sequences (i.e., % identity=number of identical positions/total number of positions (i.e., overlapping positions)×100). Preferably, the two sequences are the same length.

Several different computer programs are available to determine the degree of identity between two sequences. For instance, a comparison of sequences and determination of percent identity between two sequences can be accomplished using a mathematical algorithm. In a preferred embodiment, the percent identity between two amino acid or nucleic acid sequences is determined using the Needleman and Wunsch (J. Mol. Biol. (48): 444-453 (1970)) algorithm which has been incorporated into the GAP program in the Accelrys GCG software package (available at www.accelrys.com/products/gcg), using either a Blosum 62 matrix or a PAM250 matrix, and a gap weight of 16, 14, 12, 10, 8, 6, or 4 and a length weight of 1, 2, 3, 4, 5, or 6. These different parameters will yield slightly different results but the overall percentage identity of two sequences is not significantly altered when using different algorithms.

A sequence comparison may be carried out over the entire lengths of the two sequences being compared or over fragments of the two sequences. Typically, the comparison will be carried out over the full length of the two sequences being compared. However, sequence identity may be carried out over a region of, for example, twenty, fifty, one hundred or more contiguous amino acid residues.

“Sequence identity” as it is known in the art refers to a relationship between two or more polypeptide sequences or two or more polynucleotide sequences, namely a reference sequence and a given sequence to be compared with the reference sequence. Sequence identity is determined by comparing the given sequence to the reference sequence after the sequences have been optimally aligned to produce the highest degree of sequence similarity, as determined by the match between strings of such sequences. Upon such alignment, sequence identity is ascertained on a position-by-position basis, e.g., the sequences are “identical” at a particular position if at that position, the nucleotides or amino acid residues are identical. The total number of such position identities is then divided by the total number of nucleotides or residues in the reference sequence to give % sequence identity. Sequence identity can be readily calculated by known methods, including but not limited to, those described in Computational Molecular Biology, Lesk, A. N., ed., Oxford University Press, New York (1988), Biocomputing: Informatics and Genome Projects, Smith, D. W., ed., Academic Press, New York (1993); Computer Analysis of Sequence Data, Part I, Griffin, A. M., and Griffin, H. G., eds., Humana Press, New Jersey (1994); Sequence Analysis in Molecular Biology, von Heinge, G., Academic Press (1987); Sequence Analysis Primer, Gribskov, M. and Devereux, J., eds., M. Stockton Press, New York (1991); and Carillo, H., and Lipman, D., SIAM J. Applied Math., 48: 1073 (1988), the teachings of which are incorporated herein by reference. Preferred methods to determine the sequence identity are designed to give the largest match between the sequences tested. Methods to determine sequence identity are codified in publicly available computer programs which determine sequence identity between given sequences. Examples of such programs include, but are not limited to, the GCG program package (Devereux, J., et al., Nucleic Acids Research, 12(1):387 (1984)), BLASTP, BLASTN and FASTA (Altschul, S. F. et al., J. Molec. Biol., 215:403-410 (1990). The BLASTX program is publicly available from NCBI and other sources (BLAST Manual, Altschul, S. et al., NCBI NLM NIH Bethesda, Md. 20894, Altschul, S. F. et al., J. Molec. Biol., 215:403-410 (1990), the teachings of which are incorporated herein by reference). These programs optimally align sequences using default gap weights in order to produce the highest level of sequence identity between the given and reference sequences. As an illustration, by a polynucleotide having a nucleotide sequence having at least, for example, 95%, e.g., at least 96%, 97%, 98%, 99%, or 100% “sequence identity” to a reference nucleotide sequence, it is intended that the nucleotide sequence of the given polynucleotide is identical to the reference sequence except that the given polynucleotide sequence may include up to 5, 4, 3, 2, 1, or 0 point mutations per each 100 nucleotides of the reference nucleotide sequence. In other words, in a polynucleotide having a nucleotide sequence having at least 95%, e.g., at least 96%, 97%, 98%, 99%, or 100% sequence identity relative to the reference nucleotide sequence, up to 5%, 4%, 3%, 2%, 1%, or 0% of the nucleotides in the reference sequence may be deleted or substituted with another nucleotide, or a number of nucleotides up to 5%, 4%, 3%, 2%, 1%, or 0% of the total nucleotides in the reference sequence may be inserted into the reference sequence. These mutations of the reference sequence may occur at the 5′ or 3′ terminal positions of the reference nucleotide sequence or anywhere between those terminal positions, interspersed either individually among nucleotides in the reference sequence or in one or more contiguous groups within the reference sequence. Analogously, by a polypeptide having a given amino acid sequence having at least, for example, 95%, e.g., at least 96%, 97%, 98%, 99%, or 100% sequence identity to a reference amino acid sequence, it is intended that the given amino acid sequence of the polypeptide is identical to the reference sequence except that the given polypeptide sequence may include up to 5, 4, 3, 2, 1, or 0 amino acid alterations per each 100 amino acids of the reference amino acid sequence. In other words, to obtain a given polypeptide sequence having at least 95%, e.g., at least 96%, 97%, 98%, 99%, or 100% sequence identity with a reference amino acid sequence, up to 5%, 4%, 3%, 2%, 1%, or 0% of the amino acid residues in the reference sequence may be deleted or substituted with another amino acid, or a number of amino acids up to 5%, 4%, 3%, 2%, 1%, or 0% of the total number of amino acid residues in the reference sequence may be inserted into the reference sequence. These alterations of the reference sequence may occur at the amino or the carboxy terminal positions of the reference amino acid sequence or anywhere between those terminal positions, interspersed either individually among residues in the reference sequence or in the one or more contiguous groups within the reference sequence. Preferably, residue positions which are not identical differ by conservative amino acid substitutions. However, conservative substitutions are not included as a match when determining sequence identity.

As used herein, the term “immune response” includes innate immune responses, T-cell mediated immune responses, and/or B-cell mediated immune responses. Exemplary immune responses include T cell responses, e.g., cytokine production and cellular cytotoxicity, and B cell responses, e.g., antibody production. In addition, the term “immune response” includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+, CD8+, Th1 and Th2 cells); antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes (e.g. neutrophils).

“Parenteral” administration of an immunogenic composition includes, e.g., subcutaneous (s.c.), intravenous (i.v.), intramuscular (i.m.), or intradermal (i.d.) injection, or infusion techniques.

In the context of the field of medicine, the term “prevent” encompasses any activity which reduces the burden of mortality or morbidity from disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

A “variant” of a polypeptide according to the present invention may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

Within the meaning of the present invention, the term “conjoint administration” is used to refer to administration of a composition according to the invention and another therapeutic agent simultaneously in one composition, or simultaneously in different compositions, or sequentially (preferably, within a 24-hour period).

In accordance with the present invention there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, New York (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization (B. D. Hames & S. J. Higgins eds. (1985); Transcription and Translation (B. D. Hames & S. J. Higgins, eds. (1984); Animal Cell Culture (R. I. Freshney, ed. (1986); Immobilized Cells and Enzymes (IRL Press, (1986); B. Perbal, A Practical Guide To Molecular Cloning (1984); F. M. Ausubel et al. (eds.), Current Protocols in Molecular Biology, John Wiley & Sons, Inc. (1994); among others.

### Peptide Compositions of the Invention

Modifications of the amino acid structure of CP1 has led to the discovery of additional peptides that are able to regulate complement activation, such as C1q activity. It was previously demonstrated that modifications such as PEGylation enhanced solubility of the peptides as well as potent inhibition of biological activity compared to the parent molecule (IALILEPICCQERAA; SEQ ID NO: 1) in in vitro assays of classical complement pathway activation/inhibition, myeloperoxidase (MPO) inhibition, antioxidant activity and inhibition of NET activity. A peptide with a C-terminal monodisperse 24-mer PEGylated moiety was found to be highly soluble and had strong inhibition of the complement system (IALILEPICCQERAA-dPEG24; SEQ ID NO: 2; PA-DPEG24; PA-dPEG24). Another suitable peptide includes a sarcosine substitution at position 8 of SEQ ID NO: 2 (IALILEP(Sar)CCQERAA; SEQ ID NO: 3; PA-I8Sar; RLS-0088).

The term “peptide(s),” as used herein, refers to amino acid sequences, which may be naturally occurring, or peptide mimetics, peptide analogs and/or synthetic derivatives (including for example but not limitation PEGylated peptides) of about 15 amino acids based on SEQ ID NO: 2. In addition, the peptide may be less than about 15 amino acid residues, such as between about 10 and about 15 amino acid residues and such as peptides between about 5 to about 10 amino acid residues. Peptide residues of, for example, 5, 6, 7, 8, 9, 10, 11, 12, 13, 14, and 15 amino acids are equally likely to be peptides within the context of the present invention. Peptides can also be more than 15 amino acids, such as, for example, 16, 17, 18, 19, and 20, or more amino acids.

The disclosed peptides are generally constrained (that is, have some element of structure as, for example, the presence of amino acids that initiate a R turn or R pleated sheet, or, for example, are cyclized by the presence of disulfide bonded Cys residues) or unconstrained (that is, linear) amino acid sequences of greater than about 15 amino acid residues, about 15 amino acid residues, or less than about 15 amino acid residues.

Substitutes for an amino acid within the peptide sequence may be selected from other members of the class to which the amino acid belongs. For example, the nonpolar (hydrophobic) amino acids include alanine, leucine, isoleucine, valine, proline, phenylalanine, tryptophan, and methionine. Amino acids containing aromatic ring structures include phenylalanine, tryptophan, and tyrosine. The polar neutral amino acids include glycine, serine, threonine, cysteine, tyrosine, asparagine, and glutamine. The positively charged (basic) amino acids include arginine and lysine. The negatively charged (acidic) amino acids include aspartic acid and glutamic acid. For example, one or more amino acid residues within the sequence can be substituted by another amino acid of a similar polarity, which acts as a functional equivalent, resulting in a silent alteration.

A conservative change generally leads to less change in the structure and function of the resulting protein. A non-conservative change is more likely to alter the structure, activity, or function of the resulting protein. For example, the peptide of the present disclosure comprises one or more of the following conservative amino acid substitutions: replacement of an aliphatic amino acid, such as alanine, valine, leucine, and isoleucine, with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue.

Particularly preferred amino acid substitutions include:
a) Ala for Glu or vice versa, such that a negative charge may be reduced;
b) Lys for Arg or vice versa, such that a positive charge may be maintained;
c) Ala for Arg or vice versa, such that a positive charge may be reduced;
d) Glu for Asp or vice versa, such that a negative charge may be maintained;
e) Ser for Thr or vice versa, such that a free —OH can be maintained;
f) Gln for Asn or vice versa, such that a free NH2 can be maintained;
g) Ile for Leu or for Val or vice versa, as roughly equivalent hydrophobic amino acids;
h) Phe for Tyr or vice versa, as roughly equivalent aromatic amino acids; and
i) Ala for Cys or vice versa, such that disulphide bonding is affected.

Substitutes for an amino acid within the peptide sequence may be selected from any amino acids, including, but not limited to alanine, arginine, asparagine, aspartic acid, cysteine, glutamic acid, glutamine, glycine, histidine, isoleucine, leucine, lysine, methionine, phenylalanine, proline, pyrolysine, selenocysteine, serine, threonine, tryptophan, tyrosine, valine, N-formyl-L-methionine, sarcosine, or other N-methylated amino acids. In some embodiments, sarcosine substitutes for an amino acid within the peptide sequence. In some embodiments, a sarcosine residue replaces the isoleucine residue at position 8 of SEQ ID NO: 2.

In one embodiment, the invention discloses synthetic peptides derived from human astrovirus coat protein, the peptides comprising the amino acid sequences and modifications of SEQ ID NO: 2 and/or 3.

TABLE 1
List of Peptides of the Invention.

| SEQ | | |
|---|---|---|
| ID | | |
| NO. | Sequence | Description |
| 1 | IALILEPICCQERAA | PA (PIC1) |
| 2 | IALILEPICCQERAA-PEG24 | PA-dPEG24 |
| 3 | IALILEP(Sar)CCQERAA | PA-I8Sar |

In other embodiments, the synthetic peptides are capable of altering cytokine expression, including but not limited to models of acute lung injury (ALI). In some embodiments, the invention provides a method of altering cytokine expression comprising administering to the subject in need thereof a composition comprising a therapeutically effective amount of a synthetic peptide comprising SEQ ID NO: 2 and/or 3. In some embodiments, the synthetic peptides are capable of treating and/or preventing ALI and/or ARDS. In some embodiments, the synthetic peptides are capable of treating ocular diseases or conditions, as well as asthma. In some embodiments, the synthetic peptides are capable of modulating angiogenesis.

In other embodiments, the synthetic peptides are capable of inhibiting or altering neutrophil binding and/or adhesion. In some embodiments, the invention provides a method of inhibiting or altering neutrophil binding and/or adhesion comprising administering to the subject in need thereof a composition comprising a therapeutically effective amount of a synthetic peptide comprising SEQ ID NO: 2 and/or 3.

In other embodiments, the synthetic peptides are capable of improving neutrophil survival. In some embodiments, the invention provides a method of improving neutrophil survival comprising administering to the subject in need thereof a composition comprising a therapeutically effective amount of a synthetic peptide comprising SEQ ID NO:2 and/or 3.

In other embodiments, the synthetic peptides can bind cell surface receptors such as for example but not limitation, integrin and/or ICAMs, in vivo. In some embodiments, the method provides a method of inhibiting or altering neutrophil binding to cell surface receptors comprising administering to the subject in need thereof a composition comprising a therapeutically effective amount of a synthetic peptide comprising SEQ ID NO: 2 and/or 3.

The disclosed peptides can selectively regulate C1q and MBL activation without affecting alternative pathway activity and are, thus, ideal for preventing and treating diseases mediated by the dysregulated activation of the classical and lectin pathways. Specific blockade of classical and lectin pathways are particularly needed, as both of these pathways have been implicated in ischemia-reperfusion induced injury in many animal models. [Castellano et al., “Therapeutic targeting of classical and lectin pathways of complement protects from ischemia-reperfusion-induced renal damage.” Am J Pathol. 2010; 176(4):1648-59; Lee et al., “Early complement factors in the local tissue immunocomplex generated during intestinal ischemia/reperfusion injury.” Mol. Immunol. 2010 February; 47(5):972-81; Tjernberg, et al., “Acute antibody-mediated complement activation mediates lysis of pancreatic islets cells and may cause tissue loss in clinical islet transplantation.” Transplantation. 2008 Apr. 27; 85(8):1193-9; Zhang et al. “The role of natural IgM in myocardial ischemia-reperfusion injury.” J Mol Cell Cardiol. 2006 July; 41(1):62-7). The alternative pathway is essential for immune surveillance against invading pathogens, and humans with alternative pathway defects suffer severe bacterial infections. By binding and inactivating C1q and MBL, the peptides can efficiently regulate classical and lectin pathway activation while leaving the alternative pathway intact.

The term “regulate,” as used herein, refers to i) controlling, reducing, inhibiting or regulating the biological function of an enzyme, protein, peptide, factor, byproduct, or derivative thereof, either individually or in complexes; ii) reducing the quantity of a biological protein, peptide, or derivative thereof, either in vivo or in vitro; or iii) interrupting a biological chain of events, cascade, or pathway known to comprise a related series of biological or chemical reactions. The term “regulate” may thus be used, for example, to describe reducing the quantity of a single component of the complement cascade compared to a control sample, reducing the rate or total amount of formation of a component or complex of components, or reducing the overall activity of a complex process or series of biological reactions, leading to such outcomes as cell lysis, formation of convertase enzymes, formation of complement-derived membrane attack complexes, inflammation, or inflammatory disease. In an in vitro assay, the term “regulate” may refer to the measurable change or reduction of some biological or chemical event, but the person of ordinary skill in the art will appreciate that the measurable change or reduction need not be total to be “regulatory.”

In some embodiments, the present invention relates to therapeutically active peptides having the effects of regulating the complement system.

### Pharmaceutical Compositions of the Invention

The present disclosure provides pharmaceutical compositions capable of regulating the complement system, comprising at least one peptide, as discussed above, and at least one pharmaceutically acceptable carrier, diluent, stabilizer, or excipient. Pharmaceutically acceptable carriers, excipients, or stabilizers are nontoxic to recipients at the dosages and concentrations employed. They can be solid, semi-solid, or liquid. The pharmaceutical compositions of the present invention can be in the form of tablets, pills, powders, lozenges, sachets, cachets, elixirs, suspensions, emulsions, solutions, or syrups.

The pharmaceutical compositions of the present invention are prepared by mixing the peptide having the appropriate degree of purity with pharmaceutically acceptable carriers, diluents, or excipients. Examples of formulations and methods for preparing such formulations are well known in the art. The pharmaceutical compositions of the present invention are useful as a prophylactic and therapeutic agent for various disorders and diseases, as set forth above. In one embodiment, the composition comprises a therapeutically effective amount of the peptide. In another embodiment, the composition comprises at least one other active ingredient effective in regulating the complement system. In another embodiment, the composition comprises at least one other active ingredient effective in treating at least one disease associated with the complement system. In another embodiment, the composition comprises at least one other active ingredient effective in treating at least one disease that is not associated with the complement system. The term “therapeutically effective amount,” as used herein, refers to the total amount of each active component that is sufficient to show a benefit to the subject.

The therapeutically effective amount of the peptide varies depending on several factors, such as the condition being treated, the severity of the condition, the time of administration, the route of administration, the rate of excretion of the peptide employed, the duration of treatment, the co-therapy involved, and the age, gender, weight, and condition of the subject, etc. One of ordinary skill in the art can determine the therapeutically effective amount. Accordingly, one of ordinary skill in the art may need to titer the dosage and modify the route of administration to obtain the maximal therapeutic effect.

The effective daily dose generally is within the range of from about 0.001 to about 200 milligrams per kilogram (mg/kg) of body weight, including about 5 to about 160 mg/kg, about 10 to about 160 mg/kg, about 40 mg/kg to about 160 mg/kg, and about 40 mg/kg to about 100 mg/kg. This dose can be achieved through a 1-6 time(s) daily dosing regimen. Alternatively, optimal treatment can be achieved through a sustained release formulation with a less frequent dosing regimen. In some embodiments, the therapeutically effective amount of SEQ ID NO: 2 and/or 3 is about 10 mg/kg to about 160 mg/kg. In some embodiments, the therapeutically effective amount of SEQ ID NO: 2 and/or 3 is about 20 mg/kg to about 160 mg/kg. In some embodiments, the therapeutically effective amount of SEQ ID NO: 2 and/or 3 is about 40 mg/kg to about 160 mg/kg. In some embodiments, the therapeutically effective amount of SEQ ID NO: 2 and/or 3 is administered in at least one dose, the first dose comprising about 10 mg/kg to about 160 mg/kg SEQ ID NO: 2 and/or 3. In some embodiments, a second dose comprising a therapeutically effective amount of SEQ ID NO: 2 and/or 3 is administered, the second dose comprising about 40 mg/kg to about 60 mg/kg SEQ ID NO: 2 and/or 3. In some embodiments, the therapeutically effective amount of SEQ ID NO: 2 and/or 3 is administered in two doses, the first dose comprising about 10 mg/kg to about 160 mg/kg SEQ ID NO: 2 and/or 3 and the second dose comprising about 40 mg/kg to about 60 mg/kg SEQ ID NO: 2 and/or 3. In some embodiments, a second dose is administered 30 seconds to 3 hours after a first dose is administered.

In another aspect, the invention is a pharmaceutical composition comprising a therapeutically effective amount of SEQ ID NO: 2 and/or 3 and at least one pharmaceutically acceptable carrier, diluent, or excipient.

The compositions of the invention can comprise a carrier and/or excipient. While it is possible to use a peptide of the present invention for therapy as is, it may be preferable to administer it in a pharmaceutical formulation, e.g., in admixture with a suitable pharmaceutical excipient and/or carrier selected with regard to the intended route of administration and standard pharmaceutical practice. The excipient and/or carrier must be “acceptable” in the sense of being compatible with the other ingredients of the formulation and not deleterious to the recipient thereof. Acceptable excipients and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient and carrier can be selected with regard to the intended route of administration and standard pharmaceutical practice. Oral formulations readily accommodate additional mixtures, such as, e.g., milk, yogurt, and infant formula. Solid dosage forms for oral administration can also be used and can include, e.g., capsules, tablets, caplets, pills, troches, lozenges, powders, and granules. Non-limiting examples of suitable excipients include, e.g., diluents, buffering agents (e.g., sodium bicarbonate), preservatives, stabilizers, binders, compaction agents, lubricants, dispersion enhancers, disintegration agents, antioxidants, flavoring agents, sweeteners, and coloring agents. Those of relevant skill in the art are well able to prepare suitable solutions.

In one embodiment of any of the compositions of the invention, the composition is formulated for delivery by a route such as, e.g., oral, topical, rectal, mucosal, sublingual, nasal, naso/oro-gastric gavage, parenteral, intraperitoneal, intradermal, transdermal, intrathecal, nasal, and intracheal administration. In one embodiment of any of the compositions of the invention, the composition is in a form of a liquid, foam, cream, spray, powder, or gel. In one embodiment of any of the compositions of the invention, the composition comprises a buffering agent (e.g., sodium bicarbonate).

Administration of the peptides and compositions in the methods of the invention can be accomplished by any method known in the art. Non-limiting examples of useful routes of delivery include oral, rectal, fecal (by enema), and via naso/oro-gastric gavage, as well as parenteral, intraperitoneal, intradermal, transdermal, intrathecal, nasal, and intracheal administration. The active agent may be systemic after administration or may be localized by the use of regional administration, intramural administration, or use of an implant that acts to retain the active dose at the site of implantation.

The useful dosages of the compounds and formulations of the invention can vary widely, depending upon the nature of the disease, the patient's medical history, the frequency of administration, the manner of administration, the clearance of the agent from the host, and the like. The initial dose may be larger, followed by smaller maintenance doses. The dose may be administered as infrequently as weekly or biweekly, or fractionated into smaller doses and administered daily, semi-weekly, etc., to maintain an effective dosage level. It is contemplated that a variety of doses may be effective to achieve a therapeutic effect. While it is possible to use a compound of the present invention for therapy as is, it may be preferable to administer it in a pharmaceutical formulation, e.g., in admixture with a suitable pharmaceutical excipient, diluent or carrier selected with regard to the intended route of administration and standard pharmaceutical practice. The excipient, diluent and/or carrier must be “acceptable” in the sense of being compatible with the other ingredients of the formulation and not deleterious to the recipient thereof. Acceptable excipients, diluents, and carriers for therapeutic use are well known in the pharmaceutical art, and are described, for example, in Remington: The Science and Practice of Pharmacy. Lippincott Williams & Wilkins (A. R. Gennaro edit. 2005). The choice of pharmaceutical excipient, diluent, and carrier can be selected with regard to the intended route of administration and standard pharmaceutical practice.

Formulations suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives.

Solutions or suspensions can include any of the following components, in any combination: a sterile diluent, including by way of example without limitation, water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity, such as sodium chloride or dextrose.

In instances in which the agents exhibit insufficient solubility, methods for solubilizing agents may be used. Such methods are known to those of skill in this art, and include, but are not limited to, using co-solvents, such as, e.g., dimethylsulfoxide (DMSO), using surfactants, such as TWEEN®80, or dissolution in aqueous sodium bicarbonate. Pharmaceutically acceptable derivatives of the agents may also be used in formulating effective pharmaceutical compositions.

The composition can contain along with the active agent, for example and without limitation: a diluent such as lactose, sucrose, dicalcium phosphate, or carboxymethylcellulose; a lubricant, such as magnesium stearate, calcium stearate and talc; and a binder such as starch, natural gums, such as gum acacia gelatin, glucose, molasses, polyvinylpyrrolidone, celluloses and derivatives thereof, povidone, crospovidones and other such binders known to those of skill in the art. Liquid pharmaceutically administrable compositions can, for example, be prepared by dissolving, dispersing, or otherwise mixing an active agent as defined above and optional pharmaceutical adjuvants in a carrier, such as, by way of example and without limitation, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, such as, by way of example and without limitation, acetate, sodium citrate, cyclodextrin derivatives, sorbitan monolaurate, triethanolamine sodium acetate, triethanolamine oleate, and other such agents. Actual methods of preparing such dosage forms are known, or will be apparent, to those skilled in this art (e.g., Remington's Pharmaceutical Sciences, Mack Publishing Company, Easton, Pa., 15th Edition, 1975). The composition or formulation to be administered will, in any event, contain a quantity of the active agent in an amount sufficient to alleviate the symptoms of the treated subject.

The active agents or pharmaceutically acceptable derivatives may be prepared with carriers that protect the agent against rapid elimination from the body, such as time release formulations or coatings. The compositions may include other active agents to obtain desired combinations of properties.

Parenteral administration, generally characterized by injection, either subcutaneously, intramuscularly, or intravenously, is also contemplated herein. Injectables can be prepared in conventional forms, either as liquid solutions or suspensions, solid forms suitable for solution or suspension in liquid prior to injection, or as emulsions. Suitable excipients include, by way of example and without limitation, water, saline, dextrose, glycerol, or ethanol. In addition, if desired, the pharmaceutical compositions to be administered may also contain minor amounts of non-toxic auxiliary substances, such as wetting or emulsifying agents, pH buffering agents, stabilizers, solubility enhancers, and other such agents, such as, for example, sodium acetate, sorbitan monolaurate, triethanolamine oleate and cyclodextrins.

Lyophilized powders can be reconstituted for administration as solutions, emulsions, and other mixtures or formulated as solids or gels. The sterile, lyophilized powder is prepared by dissolving an agent provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution can be apportioned into vials for lyophilization. Each vial can contain, by way of example and without limitation, a single dosage (10-1000 mg, such as 100-500 mg) or multiple dosages of the agent. The lyophilized powder can be stored under appropriate conditions, such as at about 4° C. to room temperature. Reconstitution of this lyophilized powder with water for injection provides a formulation for use in parenteral administration.

The inventive composition or pharmaceutically acceptable derivatives thereof may be formulated as aerosols for application e.g., by inhalation or intranasally (e.g., as described in U.S. Pat. Nos. 4,044,126, 4,414,209, and 4,364,923). These formulations can be in the form of an aerosol or solution for a nebulizer, or as a microfine powder for insufflation, alone or in combination with an inert carrier such as lactose. In such a case, the particles of the formulation can, by way of example and without limitation, have diameters of less than about 50 microns, such as less than about 10 microns.

The agents may be also formulated for local or topical application, such as for application to the skin and mucous membranes (e.g., intranasally), in the form of nasal solutions, gels, creams, and lotions.

### Ophthalmic Compositions of the Invention

In some embodiments, the compositions of the inventions are formulated for ophthalmic administration, including for example topical, intravitreal, and/or intraocular administration. In some embodiments, the compositions are delivered to the ocular surface, interconnecting innervation, conjunctiva, lacrimal glands, or meibomian glands. The compositions can be in the form of eye drops, ointments, gels, foams, solutions, suspensions, and/or intraocular implants.

According to one embodiment, the invention also includes a pharmaceutical composition comprising a therapeutically effective amount of SEQ ID NO: 2 as described herein in an ophthalmically acceptable carrier and/or excipient. Such carriers include, e.g., those listed herein.

According to one embodiment the topical formulation containing the active compound can also contain a physiologically compatible vehicle, as those skilled in the ophthalmic art can select using conventional criteria. The vehicles can be selected from the known ophthalmic vehicles which include, but are not limited to, saline solution, water polyethers such as polyethylene glycol, polyvinyls such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate and salts such as sodium chloride and potassium chloride.

According to one embodiment, an ophthalmic composition is advantageously applied topically to the eye, especially in the form of a solution, a suspension, an ointment, gel, or foam. According to another embodiment, an ophthalmic composition is administered intraocularly, intravitreally or intra-aqueously via injection or implant.

The precise pharmaceutical formulation (e.g., ophthalmic composition) used in the method of the present invention will vary according to a wide range of commercial and scientific criteria. That is the skilled reader will appreciate that the above formulation of the invention described herein may contain other agents.

According to one embodiment there are used for a corresponding ophthalmic composition customary pharmaceutically acceptable excipients and additives known to the person skilled in the art, for example those of the type mentioned below, especially carriers, stabilizers, solubilizers, tonicity enhancing agents, buffer substances, preservatives, thickeners, complexing agents, and other excipients. Examples of such additives and excipients can be found in U.S. Pat. Nos. 5,134,124 and 4,906,613. Such compositions are prepared in a manner known per se, for example by mixing the active ingredient with the corresponding excipients and/or additives to form corresponding ophthalmic compositions. The active ingredient is preferably administered in the form of eye drops, the active ingredient being conventionally dissolved, for example, in a carrier. The solution is, where appropriate, adjusted and/or buffered to the desired pH and, where appropriate, a stabilizer, a solubilizer or a tonicity enhancing agent is added. Where appropriate, preservatives and/or other excipients are added to an ophthalmic composition.

Carriers used in accordance with the present invention are typically suitable for topical or general administration, and are for example water, mixtures of water and water-miscible solvents, such as C1-C7-alkanols, vegetable oils or mineral oils comprising from 0.5 to 5% by weight hydroxyethylcellulose, ethyl oleate, carboxymethylcellulose, polyvinyl-pyrrolidone and other non-toxic water-soluble polymers for ophthalmic uses, such as, for example, cellulose derivatives, such as methylcellulose, alkali metal salts of carboxymethylcellulose, hydroxymethylcellulose, hydroxyethylcellulose, methylhydroxypropylcellulose and hydroxypropylcellulose, acrylates or methacrylates, such as salts of polyacrylic acid or ethyl acrylate, polyacrylamides, natural products, such as gelatin, alginates, pectins, tragacanth, karaya gum, xanthan gum, carrageenin, agar and acacia, starch derivatives, such as starch acetate and hydroxypropyl starch, and also other synthetic products, such as polyvinyl alcohol, polyvinylpyrrolidone, polyvinyl methyl ether, polyethylene oxide, preferably cross-linked polyacrylic acid, such as neutral Carbopol, or mixtures of those polymers. Preferred carriers are water, cellulose derivatives, such as methylcellulose, alkali metal salts of carboxymethylcellulose, hydroxymethylcellulose, hydroxyethylcellulose, methylhydroxypropylcellulose and hydroxypropylcellulose, neutral Carbopol, or mixtures thereof.

According to one embodiment the solubilizers used for an ophthalmic composition of the present invention are, for example, tyloxapol, fatty acid glycerol poly-lower alkylene glycol esters, fatty acid poly-lower alkylene glycol esters, polyethylene glycols, glycerol ethers or mixtures of those compounds. The amount added is typically sufficient to solubilize the active ingredient. For example, the concentration of the solubilizer is from 0.1 to 5000 times the concentration of the active ingredient. Lower alkylene means linear or branched alkylene with up to and including 7 C-atoms. Examples are methylene, ethylene, 1,3-propylene, 1,2-propylene, 1,5-pentylene, 2,5-hexylene or 1,7-heptylene. Lower alkylene is preferably linear or branched alkylene with up to and including 4 C-atoms.

Examples of buffer substances are acetate, ascorbate, borate, hydrogen carbonate/carbonate, citrate, gluconate, lactate, phosphate, propionate, and TRIS (tromethamine) buffers. Tromethamine and borate buffer are preferred buffers. The amount of buffer substance added is, for example, that necessary to ensure and maintain a physiologically tolerable pH range. The pH range is typically in the range of from 5 to 9, preferably from 6 to 8.2 and more preferably from 6.8 to 8.1.

Tonicity enhancing agents are, for example, ionic compounds, such as alkali metal or alkaline earth metal halides, such as, for example, CaCl₂), KBr, KCl, LiCl, NaBr, NaCl, or boric acid. Non-ionic tonicity enhancing agents are, for example, urea, glycerol, sorbitol, mannitol, propylene glycol, or dextrose. For example, sufficient tonicity enhancing agent is added to impart to the ready-for-use ophthalmic composition an osmolality of approximately from 50 to 1000 mOsmol, preferred from 100 to 400 mOsmol, more preferred from 200 to 400 mOsmol and even more preferred from 280 to 350 mOsmol.

Examples of preservatives are quaternary ammonium salts, such as cetrimide, benzalkonium chloride or benzoxonium chloride, alkyl-mercury salts of thiosalicylic acid, such as, for example, thimerosal, phenylmercuric nitrate, phenylmercuric acetate or phenylmercuric borate, parabens, such as, for example, methylparaben or propylparaben, alcohols, such as, for example, chlorobutanol, benzyl alcohol or phenyl ethanol, guanidine derivatives, such as, for example, chlorohexidine or polyhexamethylene biguanide, or sorbic acid. Preferred preservatives are cetrimide, benzalkonium chloride, benzoxonium chloride and parabens. Where appropriate, a sufficient amount of preservative is added to the ophthalmic composition to ensure protection against secondary contaminations during use caused by bacteria and fungi.

According to one embodiment the ophthalmic compositions may comprise further non-toxic excipients, such as, for example, emulsifiers, wetting agents, or fillers, such as, for example, the polyethylene glycols designated 200, 300, 400 and 600, or Carbowax designated 1000, 1500, 4000, 6000 and 10 000. Other excipients that may be used if desired are listed below but they are not intended to limit in any way the scope of the possible excipients. They are especially complexing agents, such as disodium-EDTA or EDTA, antioxidants, such as ascorbic acid, acetylcysteine, cysteine, sodium hydrogen sulfite, butyl-hydroxyanisole, butyl-hydroxy-toluene or α-tocopherol acetate; stabilizers, such as a cyclodextrin, thiourea, thiosorbitol, sodium dioctyl sulfosuccinate or monothioglycerol; or other excipients, such as, for example, lauric acid sorbitol ester, triethanol amine oleate or palmitic acid ester. Preferred excipients are complexing agents, such as disodium-EDTA and stabilizers, such as a cyclodextrin. The amount and type of excipient added is in accordance with the particular requirements and is generally in the range of from approximately 0.0001 to approximately 90% by weight. A cyclodextrin is composed of several glucose units which have three free hydroxy groups per glucose. The amount of a cyclodextrin used in accordance with one embodiment may preferably range from 0.01-20% by weight, more preferably from 0.1-15% by weight and even more preferably from 1-10% by weight.

According to one embodiment the present invention relates also to an ophthalmic composition, which comprises a therapeutically effective amount of SEQ ID NO: 2 as described herein a carrier, a solubilizer and another therapeutically effective pharmaceutical agent which may be, for example, an antibiotic, an antiallergic, an anesthetic, another antiphlogistic, a corticosteroid, an agent suitable for lowering intra-ocular pressure, or another drug.

The ophthalmic compositions used in the methods of the invention are preferably prepared using a physiological saline solution as a vehicle. The pH of the ophthalmic composition may be maintained at a substantially neutral pH (for example, about 7.4, in the range of about 6.5 to about 7.4, etc.) with an appropriate buffer system as known to one skilled in the art (for example, acetate buffers, citrate buffers, phosphate buffers, borate buffers).

Topical Formulations

Ophthalmic ointments tend to keep an active agent in contact with the eye longer than suspensions and certainly solutions. Most ointments tend to blur vision, as they are not removed easily by the tear fluid. Thus, ointments are generally used at night as adjunctive therapy to eye drops used during the day.

Oleaginous ointment bases of inventive compositions are mixtures of mineral oil, petrolatum and lanolin all have a melting point close to body temperature. In the case of the inventive compounds, the compositions may include mineral oil, petrolatum, or lanolin. According to one embodiment preferred compositions can include a combination of petrolatum, mineral oil, and lanolin. Another preferred composition is an ointment combination containing white petrolatum, mineral oil, and lanolin (anhydrous).

Other exemplary topical formulations include eye drops, inserts, eye packs, impregnated contact lenses, pump delivery systems, dimethylsulfoxide (DMSO)-based solutions and/or suspensions, and liposomes.

Eye drops may be prepared by dissolving the active ingredient in a sterile aqueous solution such as physiological saline, buffering solution, etc., or by combining powder compositions to be dissolved before use. Other vehicles may be chosen, as is known in the art, including but not limited to: balance salt solution, saline solution, water soluble polyethers such as polyethylene glycol, polyvinyls, such as polyvinyl alcohol and povidone, cellulose derivatives such as methylcellulose and hydroxypropyl methylcellulose, petroleum derivatives such as mineral oil and white petrolatum, animal fats such as lanolin, polymers of acrylic acid such as carboxypolymethylene gel, vegetable fats such as peanut oil and polysaccharides such as dextrans, and glycosaminoglycans such as sodium hyaluronate. If desired, additives ordinarily used in the eye drops can be added. Such additives include isotonizing agents (e.g., sodium chloride, etc.), buffer agent (e.g., boric acid, sodium monohydrogen phosphate, sodium dihydrogen phosphate, etc.), preservatives (e.g., benzalkonium chloride, benzethonium chloride, chlorobutanol, etc.), thickeners (e.g., saccharide such as lactose, mannitol, maltose, etc.; e.g., hyaluronic acid or its salt such as sodium hyaluronate, potassium hyaluronate, etc.; e.g., mucopolysaccharide such as chondroitin sulfate, etc.; e.g., sodium polyacrylate, carboxyvinyl polymer, crosslinked polyacrylate, polyvinyl alcohol, polyvinyl pyrrolidone, methyl cellulose, hydroxy propyl methylcellulose, hydroxyethyl cellulose, carboxymethyl cellulose, hydroxy propyl cellulose or other agents known to those skilled in the art).

The solubility of the components of the present compositions may be enhanced by a surfactant or other appropriate co-solvent in the composition. Such cosolvents include polysorbate 20, 60, and 80, Pluronic F68, F-84 and P-103, cyclodextrin, or other agents known to those skilled in the art. Such co-solvents may be employed at a level of from about 0.01% to 2% by weight.

The composition of the invention can be formulated as a sterile unit dose type containing no preservatives. The compositions of the invention may be packaged in multidose form. Preservatives may be preferred to prevent microbial contamination during use. Suitable preservatives include: benzalkonium chloride, thimerosal, chlorobutanol, methyl paraben, propyl paraben, phenylethyl alcohol, edetate disodium, sorbic acid, Onamer M, or other agents known to those skilled in the art. In the prior art ophthalmic products, such preservatives may be employed at a level of from 0.004% to 0.02%. In the compositions of the present application the preservative, preferably benzalkonium chloride, may be employed at a level of from 0.001% to less than 0.01%, e.g., from 0.001% to 0.008%, preferably about 0.005% by weight. It has been found that a concentration of benzalkonium chloride of 0.005% may be sufficient to preserve the compositions of the present invention from microbial attack.

The formulations of the invention may be administered several drops per time, one to four drops, preferably one to three drops, more preferably one to two drops, and most preferably one drop per day. Alternatively, the formulations of the invention may be applied or sprayed several times a day, preferably one to six times, more preferably one to four times, and most preferably once a day.

Conjunctival/Scleral Formulations

The topical conjunctival route of entry enables penetration of drugs into the anterior segment. Furthermore, topically applied drugs have been shown to have access to the sclera from the conjunctiva. The sclera has been shown to be readily permeable to even large molecular weight compounds (150 kD). Topical solutions, suspensions, gels, or ointments comprising SEQ ID NO:2 and/or 3 are suitable formulations for topical conjunctival and scleral application. It is also possible to administer the pharmaceutical compositions via subconjunctival injection.

Intraocular/Intravitreal Formulations

The pharmaceutical compositions of the invention may be formulated to be administered intraocularly or intravitreally, by means of injection (e.g., periocular, subconjunctival, intra-aqueous, intraocular, or intravitreal injection) or introduction of a suitable implant (e.g., intracorneal or intraocular implant).

Implantable Formulations

In one embodiment, implants comprising the ocular compositions of the present invention are formulated with PIC1 peptides entrapped within a biocompatible, biodegradable/bio-erodible polymer matrix. Release of the agent is achieved by erosion of the polymer followed by exposure of previously entrapped agent particles to the vitreous, and subsequent dissolution and release of agent. The release kinetics achieved by this form of drug release are different than that achieved through formulations which release drug through polymer swelling, such as with hydrogels such as methylcellulose. In that case, the drug is not released through polymer erosion, but through polymer swelling, which releases drug as liquid diffuses through the pathways exposed. The parameters which determine the release kinetics include the size of the drug particles, the water solubility of the drug, the ratio of drug to polymer, the method of manufacture, the surface area exposed, and the erosion rate of the polymer.

Diffusion of the PIC1 peptide(s) from the implant may also be controlled by the structure of the implant. For example, diffusion of the PIC1 peptide(s) from the implant may be controlled by means of a membrane affixed to the polymer layer comprising the drug. The membrane layer will be positioned intermediate to the polymer layer comprising the peptide(s) and the desired site of therapy. The membrane may be composed of any of the biocompatible materials indicated above, the presence of agents in addition to the peptide(s) present in the polymer, the composition of the polymer comprising the PIC1 peptide(s), the desired rate of diffusion and the like. For example, the polymer layer will usually comprise a very large amount of peptide(s) and will typically be saturated. Such PIC1 peptide(s)-saturated polymers may generally release the peptide(s) at a very high rate. In this situation, the release of the peptide(s) may be slowed by selecting a membrane which is of a lower peptide(s) permeability than the polymer. Due to the lower peptide(s) permeability of the membrane, the peptide(s) will remain concentrated in the polymer and the overall rate of diffusion will be determined by the peptide(s) permeability of the membrane. Therefore, the rate of release of the peptide(s) from the implant is reduced, providing for a more controlled and extended delivery of the peptide(s) to the site of therapy.

Ocular Administration

Administration of the ophthalmic compositions of the invention may by intraocular injection, although other modes of administration may be effective. Typically, ophthalmic composition will be delivered intraocularly (by chemical delivery system or invasive device) to an individual. However, the invention is not limited to intraocular delivery in that it also includes topically (extraocular application) or systemically (e.g., oral, or other parenteral route such as for example subcutaneous administration) provided that a sufficient amount of the peptide within cells or tissue located in an eye or adjacent an eye achieves contact with the site of the ophthalmic condition. Parenteral administration is used in appropriate circumstances apparent to the practitioner. Preferably, the ophthalmic compositions are administered in unit dosage forms suitable for single administration of precise dosage amounts.

As mentioned above, delivery to areas within the eye, in situ can be accomplished by injection, cannula or other invasive device designed to introduce precisely metered amounts of a desired ophthalmic composition to a particular compartment or tissue within the eye (e.g., posterior chamber or retina). An intraocular injection may be into the vitreous (intravitreal), or under the conjunctiva (subconjunctival), or behind the eye (retrobulbar), into the sclera, or under the Capsule of Tenon (sub-Tenon), and may be in a depot form. Other intraocular routes of administration and injection sites and forms are also contemplated and are within the scope of the invention.

Topical application of ophthalmic composition of the invention for the treatment or prevention of ophthalmic disorders may be as ointment, gel, foam, or eye drops. Preferably a penetrating composition comprising the PIC1 peptide(s) is used. The topical ophthalmic composition may further be an in situ gellable aqueous formulation. Such a formulation comprises a gelling agent in a concentration effective to promote gelling upon contact with the eye or with lacrimal fluid in the exterior of the eye. Suitable gelling agents include, but are not limited to, thermosetting polymers such as tetra-substituted ethylene diamine block copolymers of ethylene oxide and propylene oxide (e.g., poloxamine); polycarbophil; and polysaccharides such as gellan, carrageenan (e.g., kappa-carrageenan and iota-carrageenan), chitosan and alginate gums.

The amount of the PIC1 peptide(s) to be administered and the concentration of the compound in the topical ophthalmic composition used in the method depend upon the diluent, delivery system or selected device, the clinical condition of the patient, the side effects, and the stability of the compound in the formulation. Thus, the physician employs the appropriate preparation containing the appropriate concentration of the peptide(s) and selects the amount of formulation administered, depending upon clinical experience with the patient in question or with similar patients.

Slow or extended-release delivery systems include any of a number of biopolymers (biological-based systems), systems employing liposomes, colloids, resins, and other polymeric delivery systems or compartmentalized reservoirs, can be utilized with the compositions described herein to provide a continuous or long-term source of therapeutic compound.

The skilled reader will appreciate that the duration over which any of the ophthalmic compositions used in the method of the invention will dwell in the ocular environment will depend, inter alia, on such factors as the physicochemical and/or pharmacological properties of the compounds employed in the formulation, the concentration of the compound employed, the bioavailability of the compound, the disease to be treated, the mode of administration and the preferred longevity of the treatment.

The frequency of treatment according to the method of the invention is determined according to the disease being treated, the deliverable concentration of the PIC1 peptide(s) and the method of delivery. If delivering the peptide(s) by intravitreal injection, the dosage frequency may be monthly. Preferably, the dosage frequency is every three months. The frequency of dosage may also be determined by observation, with the dosage being delivered when the previously delivered peptide(s) is visibly cleared. Once a therapeutic result is achieved, the peptide(s) can be tapered or discontinued. Occasionally, side effects warrant discontinuation of therapy. In general, an effective amount of the compound is that which provides either subjective relief of symptoms or an objectively identifiable improvement as noted by the clinician or other qualified observer.

### Nasal Compositions of the Invention

In some embodiments, the compositions of the inventions are formulated for nasal administration, including for example inhalation, insufflation, or nebulization. The compositions can be in the form of, e.g., nose drops, nose sprays, and formulations suitable for inhalation, insufflation, and/or nebulization.

According to one embodiment, the invention also includes a pharmaceutical composition comprising a therapeutically effective amount of SEQ ID NO: 2 and/or 3 as described herein in a nasally acceptable carrier and/or excipient. Such carriers include, e.g., those listed herein.

### Nasal Administration

Administration of the nasal compositions of the invention may be by nasal drops, sprays, inhalable formulations, and nebulized formulations, although other modes of administration may be effective. However, the invention is not limited to nasal delivery in that it also includes topically (intranasal application) or systemically (e.g., oral, or other parenteral route such as for example subcutaneous administration) provided that a sufficient amount of the peptide within cells or tissue located in the nose achieves therapeutic efficacy. Parenteral administration is used in appropriate circumstances apparent to the practitioner. Preferably, the nasal compositions are administered in unit dosage forms suitable for single administration of precise dosage amounts.

As mentioned above, delivery to areas within the nose, in situ can be accomplished by sprays, drops, or an inhaler or nebulizer device designed to introduce precisely metered amounts of a desired nasal composition to the nasal passages. Other intranasal routes of administration and forms are also contemplated and are within the scope of the invention.

The amount of the PIC1 peptide(s) to be administered and the concentration of the compound in the nasal composition used in the method depend upon the diluent, delivery system or selected device, the clinical condition of the patient, the side effects, and the stability of the compound in the formulation. Thus, the physician employs the appropriate preparation containing the appropriate concentration of the peptide(s) and selects the amount of formulation administered, depending upon clinical experience with the patient in question or with similar patients.

The skilled reader will appreciate that the duration over which any of the nasal compositions used in the method of the invention will dwell in the nasal environment will depend, inter alia, on such factors as the physicochemical and/or pharmacological properties of the compounds employed in the formulation, the concentration of the compound employed, the bioavailability of the compound, the disease to be treated, the mode of administration and the preferred longevity of the treatment.

The frequency of treatment according to the method of the invention is determined according to the disease being treated, the deliverable concentration of the PIC1 peptide(s) and the method of delivery. If delivering the peptide(s) by nasal inhalation or insufflation, the dosage frequency may be monthly. Preferably, the dosage frequency is every three months. The frequency of dosage may also be determined by observation, with the dosage being delivered when the previously delivered peptide(s) is visibly cleared. Once a therapeutic result is achieved, the peptide(s) can be tapered or discontinued. Occasionally, side effects warrant discontinuation of therapy. In general, an effective amount of the compound is that which provides either subjective relief of symptoms or an objectively identifiable improvement as noted by the clinician or other qualified observer.

Combination Therapies

A further embodiment of the invention provides a method of regulating the complement system, comprising administering to a subject a pharmaceutical composition of the present invention. While the pharmaceutical compositions of the present invention can be administered as the sole active pharmaceutical agent, they can also be used in combination with one or more therapeutic or prophylactic agent(s) that is(are) effective for regulating the complement system. In this aspect, the method of the present invention comprises administrating a pharmaceutical composition of the present invention before, concurrently, and/or after one or more additional therapeutic or prophylactic agents effective in regulating the complement system.

The pharmaceutical compositions of the present invention can be administered with additional agent(s) in combination therapy, either jointly or separately, or by combining the pharmaceutical compositions and the additional agent(s) into one composition. The dosage is administered and adjusted to achieve maximal regulation of the complement system. For example, both the pharmaceutical compositions and the additional agent(s) are usually present at dosage levels of between about 10% and about 150%, more preferably, between about 10% and about 80%, of the dosage normally administered in a mono-therapy regimen.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1: Effect of PA-dPEG24 on Cytokine Expression and Lung Injury in ALI

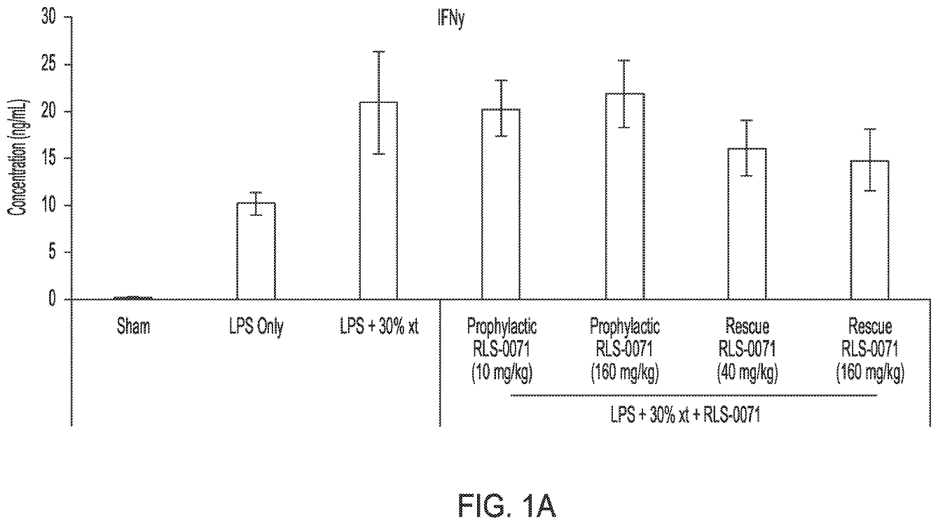

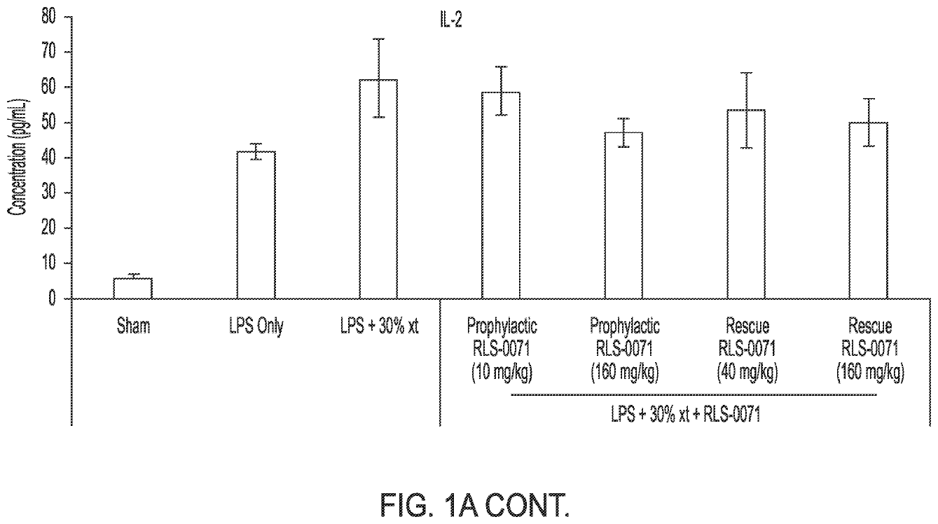

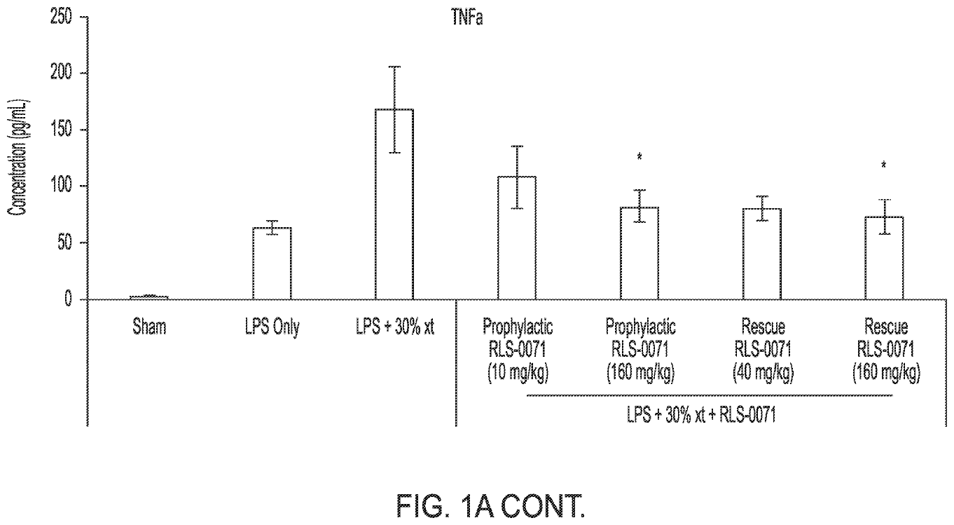

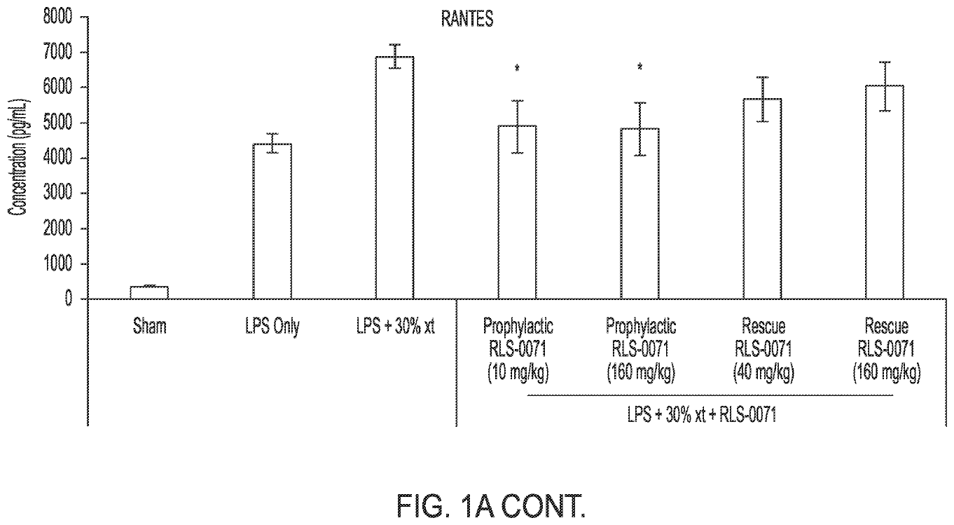

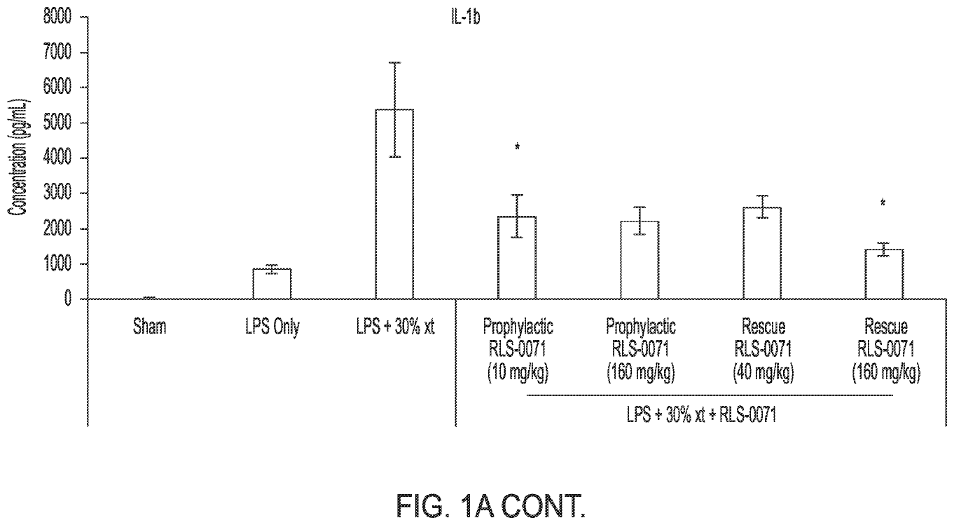

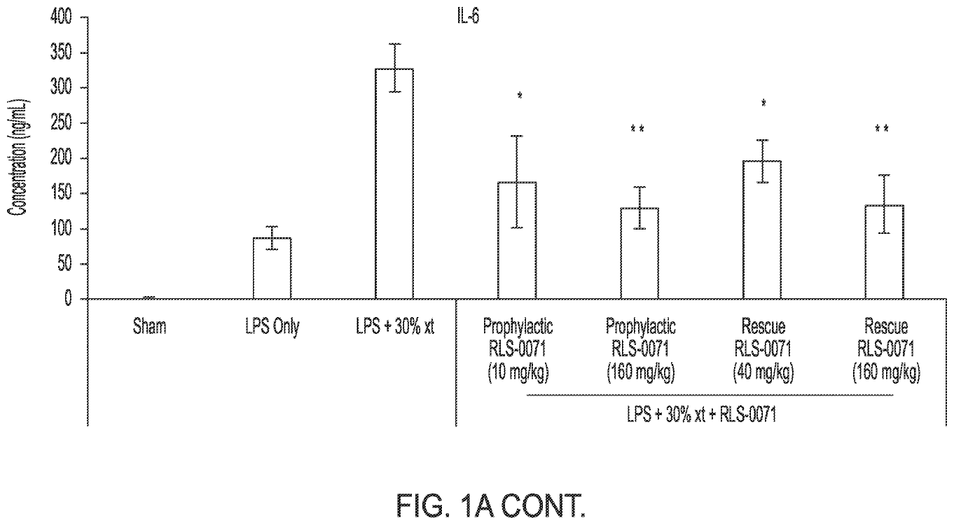

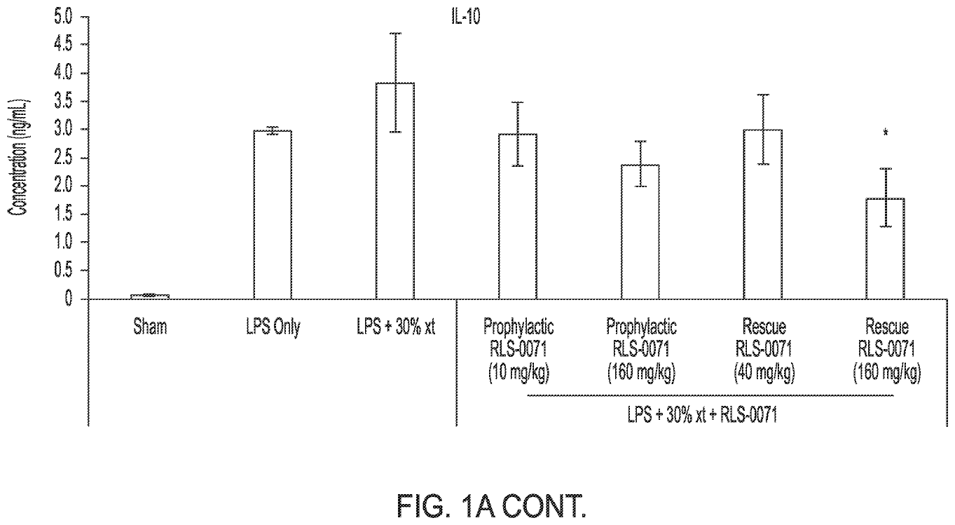

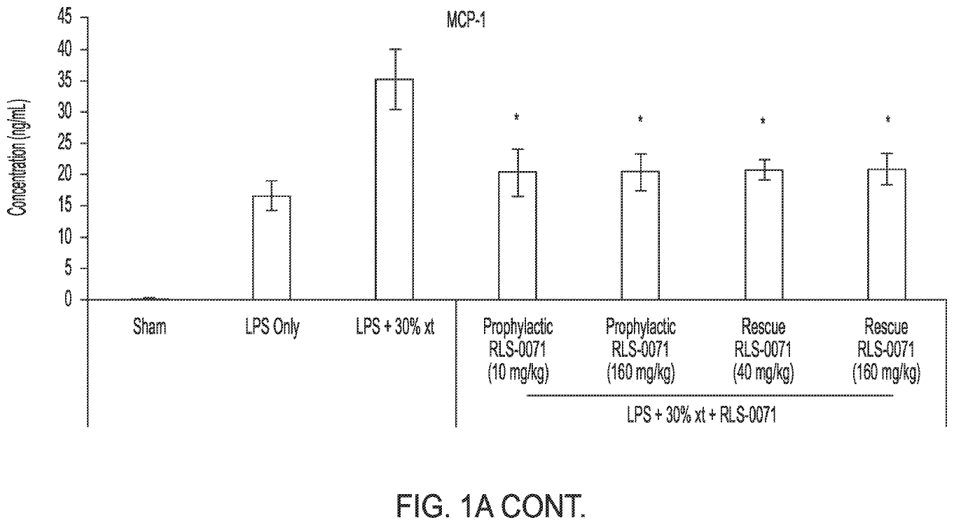

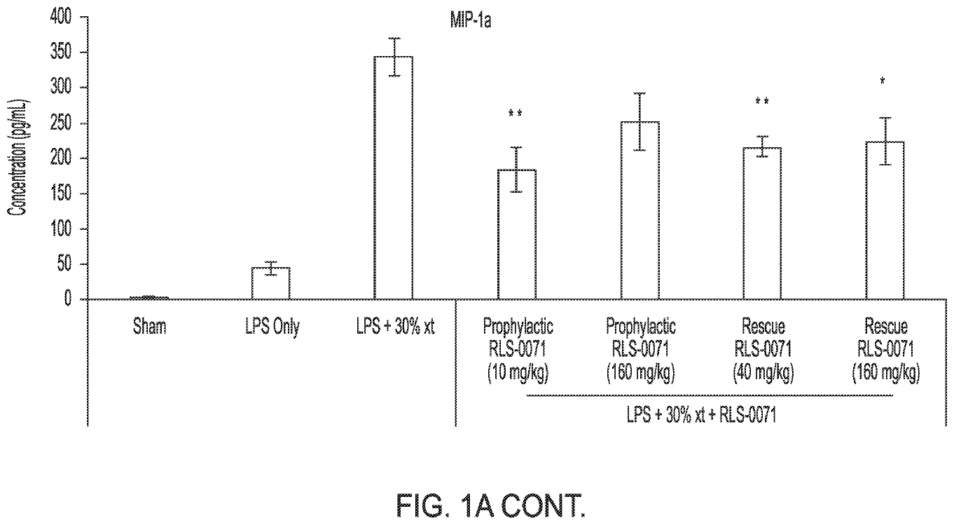

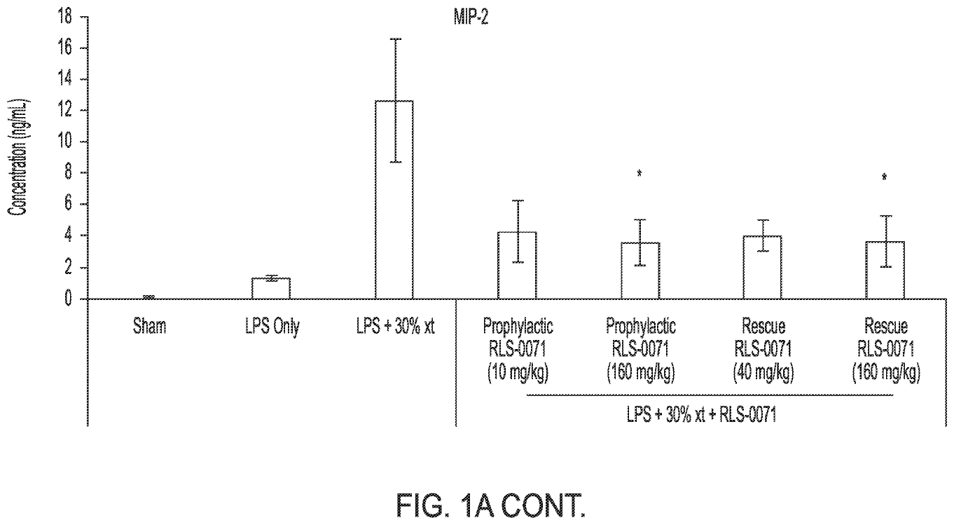

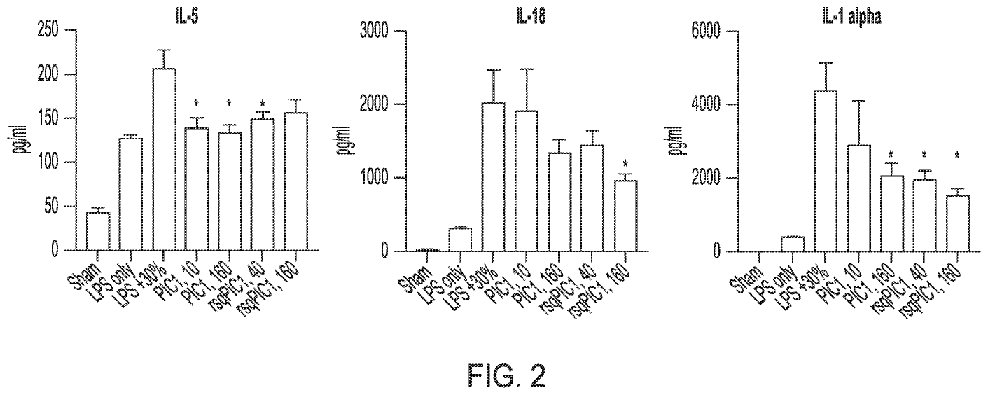

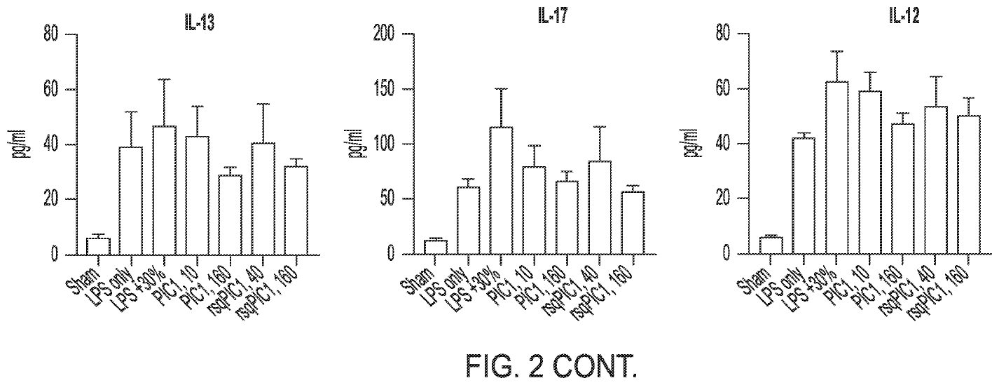

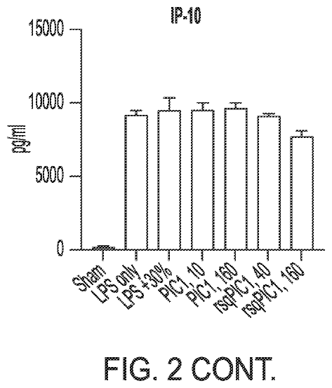

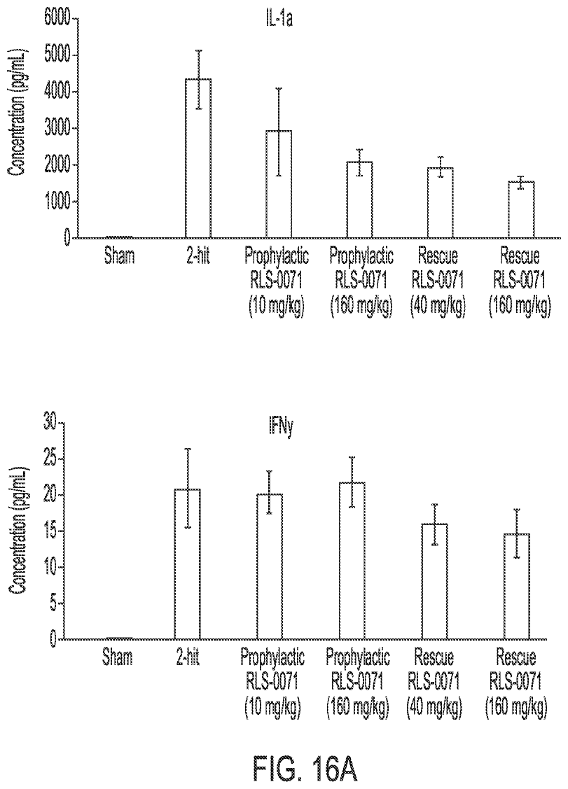

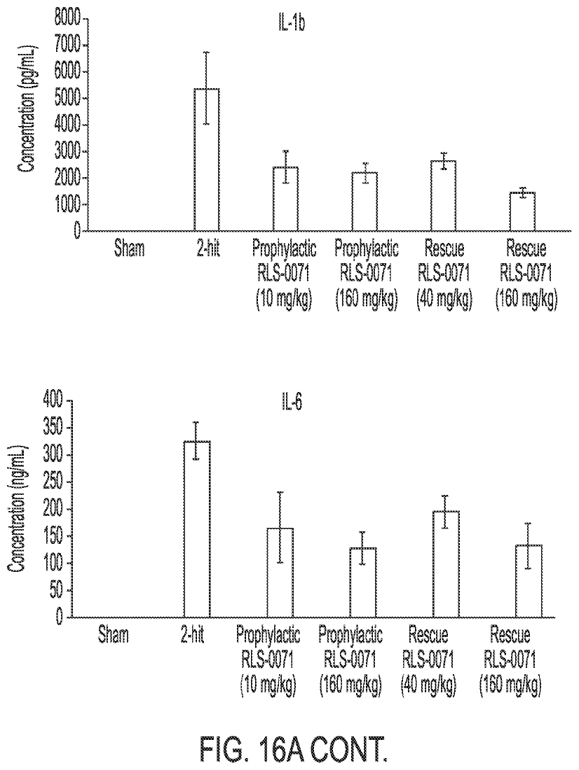

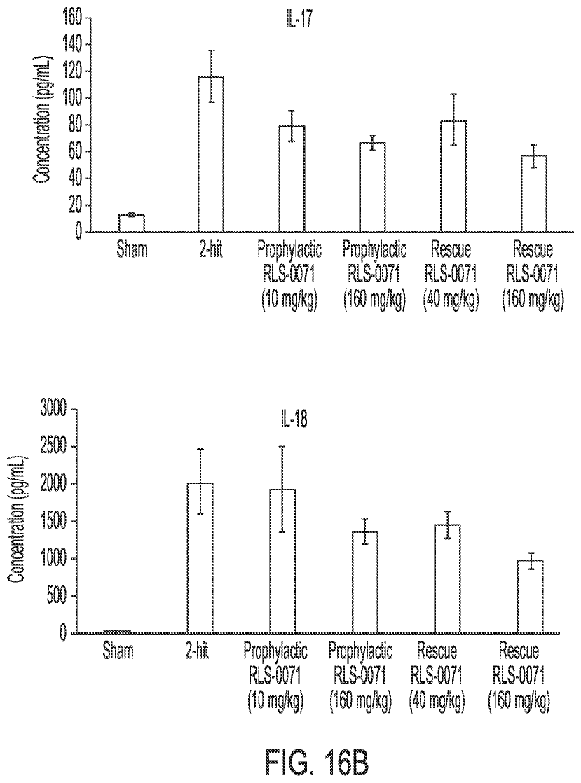

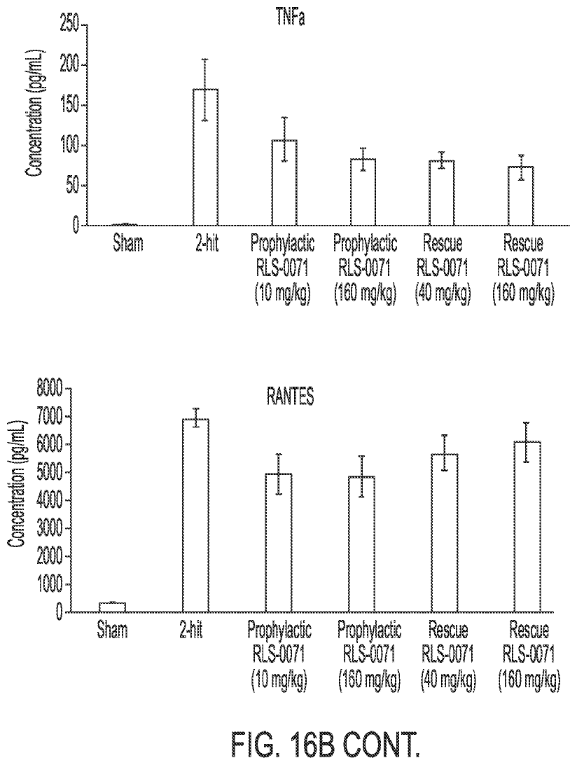

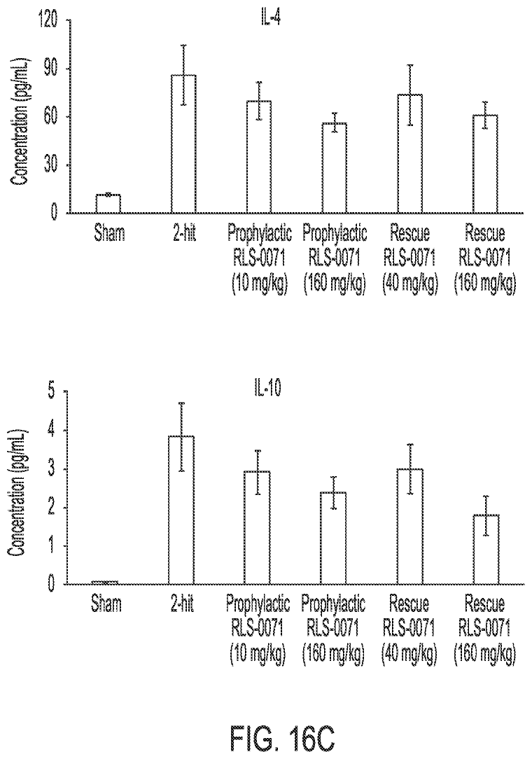

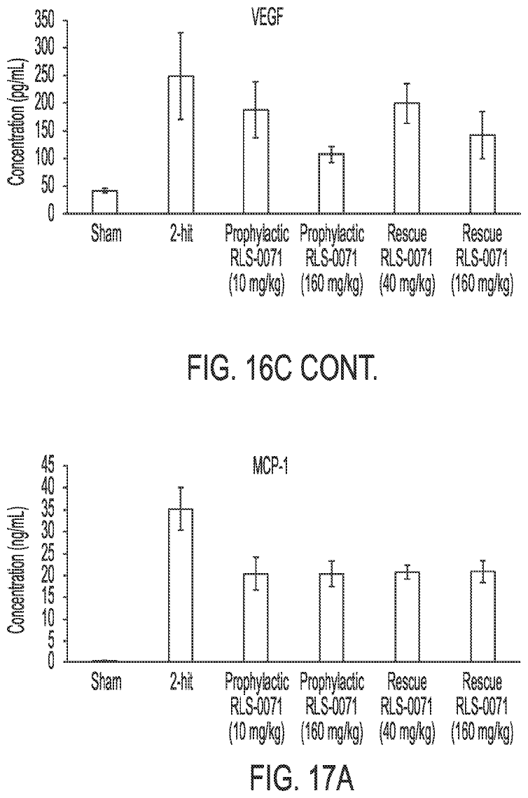

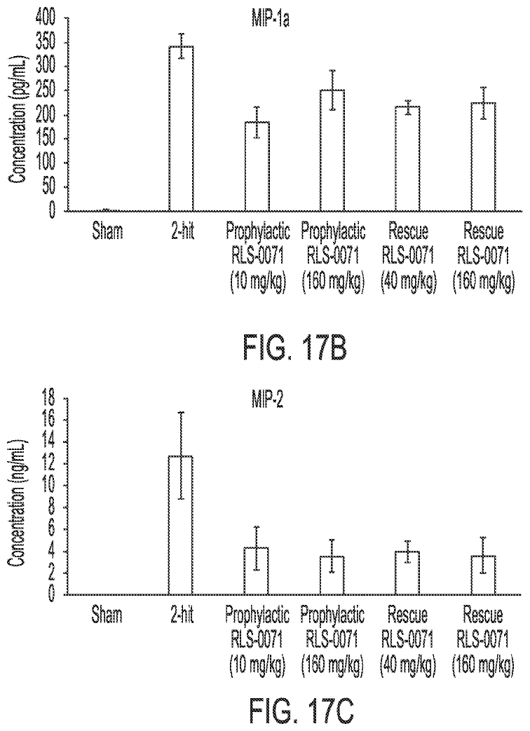

The acute lung injury that manifests in severe cases of COVID-19 has been demonstrated to result from the host immune response involving cytokine storm (Mehta et al., 2020), and it has been postulated that NETosis is a key driver of acute lung injury (ALI) in COVID-19 patients through direct lung injury as well as contributing to cytokine production (Barnes et al., 2020). To determine the effect of a single dose of 10 and 160 mg/kg of SEQ ID NO: 2 administered either before or a single dose of 40 and 160 mg/kg administered at different times after transfusion on cytokine levels, terminal blood samples were taken. Plasma was isolated from the blood samples and analyzed for the following inflammatory cytokines by xMAP bead-based immunoassay: IFNgamma, IL-6, IL-2, IL-10, TNFalpha, MCP-1 (CCL-2), RANTES (CCL-5), MIPlalpha (CCL-3), IL-1beta, and MIP-2 (CXCL2). This dose of SEQ ID NO: 2 was tested in a two hit ALI rat model (Rivera et al., 2020). In this model, which is an adaptation of the established two-hit ALI model (Silliman et al., 1997; Silliman, 2006), adolescent male Wistar rats are injected with lipopolysaccharide (LPS) (first hit) to prime neutrophils followed 30 minute later by transfusion of 30% incompatible erythrocytes (second hit) to initiate complement activation. Most importantly, this two hit ALI model produces extremely robust responses across a broad spectrum of pro-inflammatory cytokines replicating a cytokine storm. A dose of 10 or 160 mg/kg of SEQ ID NO: 2 given before transfusion or a dose of 40 or 160 mg/kg given after transfusion modulated cytokine levels with either significant pro-inflammatory cytokine reduction or a trend toward reduced levels (FIG. 1). Analysis of other inflammatory cytokines by xMAP bead-based immunoassay included: IL-5, IL-18, IL-1alpha, IL-13, IL-17, IL-12, and IP-10 (FIG. 2). A PA-dPEG24 dose of 10 or 160 mg/kg given before transfusion or a dose of 40 or 160 mg/kg given after transfusion modulated cytokine levels with either significant pro-inflammatory cytokine reduction or a trend toward reduced levels. In a second round of experiments, plasma samples were analyzed for the following experimental groups: sham, 1-hit, 2-hit, 2-hit+10 mg/kg prophylactic dose RLS-0071, 2-hit+160 mg/kg prophylactic dose RLS-0071, 2-hit+40 mg/kg rescue dose RLS-0071 at 30 seconds, and 2-hit+160 mg/kg rescue dose RLS-0071 at 30 seconds (FIG. 16A-16C and FIG. 17A-17C). For each cytokine reported, two replicates were run for each animal. Data are means and standard error of the means.

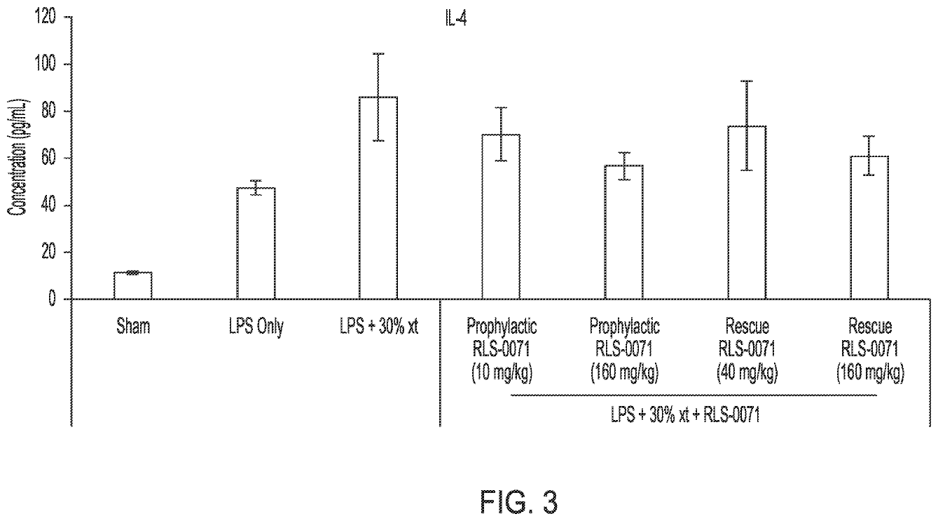

Importantly, in contrast to the reduction of inflammatory Th1 and Th17 cytokines shown in FIGS. 1 and 2, PA-dPEG24 did not significantly decrease levels of the anti-inflammatory Th2 cytokine IL-4 (FIG. 3). Th2 cells promote alternative activation of M2 macrophages involved in reduction of pathological inflammation and counteract the Th1 responses.

These findings surprisingly showed that PA-dPEG24 can modulate the pro-inflammatory responses in ALI. It was unexpected that PA-dPEG24 could modulate inflammatory cytokine levels in this ALI animal model. The observation that both lung damage as assessed by histology and inflammatory cytokine levels are reduced by both prophylactic and rescue dosing of PA-DPEG24 suggests that this molecule can reduce the multiple inflammatory pathways that contribute to ALI.

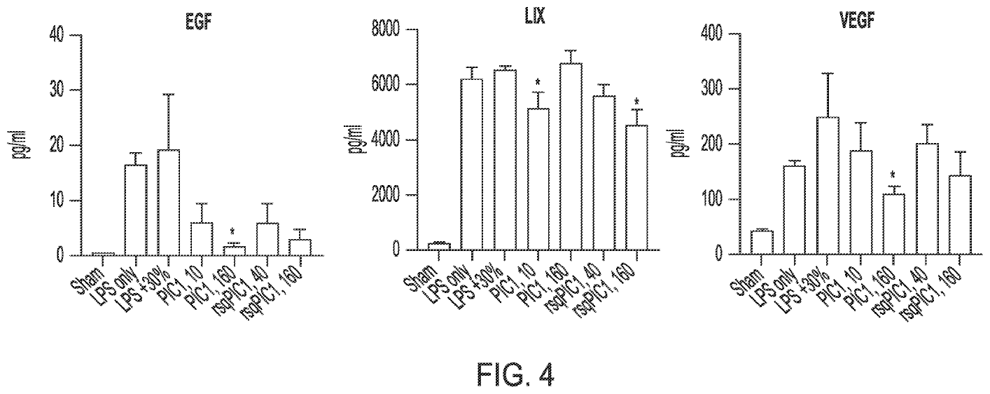

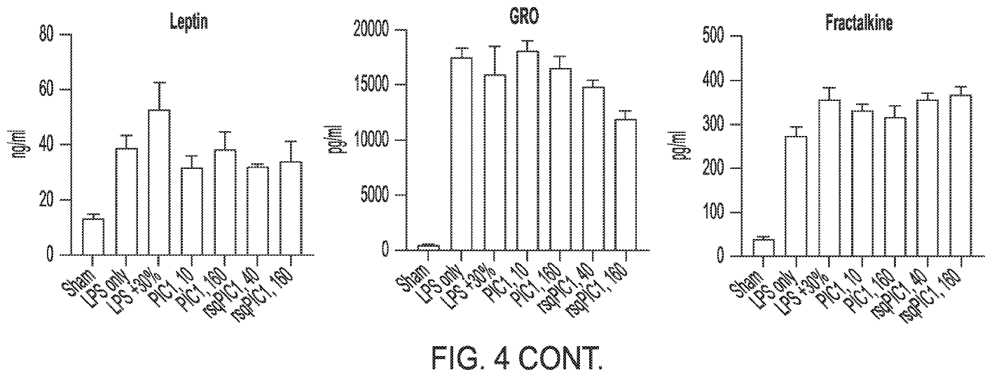

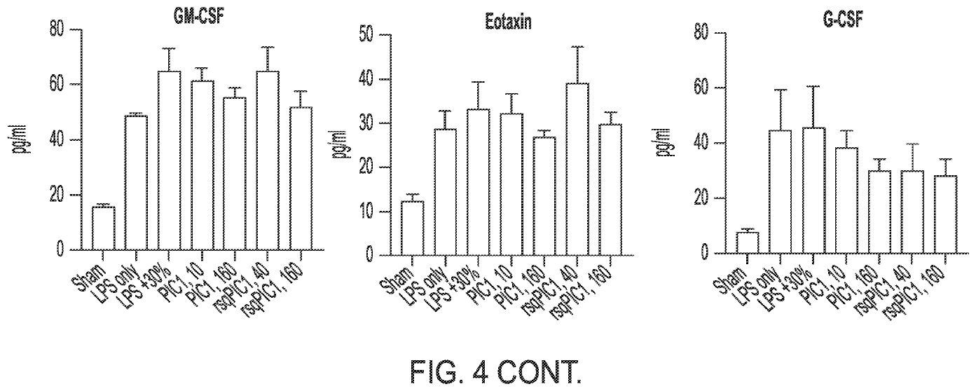

In addition to the cytokines shown above, a dose of PA-dPEG24 at 10 or 160 mg/kg given before transfusion or a dose of 40 or 160 mg/kg given after transfusion modulated other cytokine and growth factor levels with either significant reduction or a trend toward reduced levels (FIG. 4).

### RLS-0071 Reduces Neutrophil-Mediated ALI

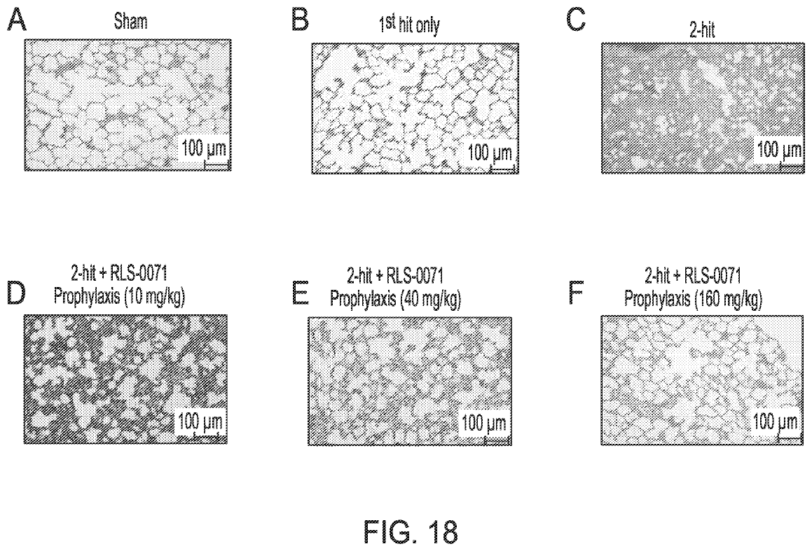

The inventors' previously developed two-hit ALI model is initiated by infusion of LPS (first hit) into Wistar rats followed 30 minutes later with transfusion of 30% incompatible erythrocytes (second hit) and sacrifice of the animals 4 hours later. Lungs of the animals showed dramatic neutrophil-mediated ALI as well as robust complement activation and NETosis as measured by C5a levels and free DNA in the bloodstream, respectively. To evaluate the ability of RLS-0071 to mitigate lung damage in this model, animals were treated with a single prophylactic dose of RLS-0071 administered 2 minutes prior to the second hit or as a rescue dose at various times after the second hit. Lungs were isolated from animals four hours after the second hit and tissues evaluated by H&E staining. Sham animals (FIG. 18A) or animals receiving the first hit of LPS alone (FIG. 18B) displayed normal lung tissue architecture whereas animals that received the 2-hit insult showed striking lung damage mediated by substantial neutrophil infiltration into the alveolar walls (FIG. 18C). In contrast, animals receiving prophylactic doses of RLS-0071 at 10, 40 or 160 mg/kg 2 minutes before incompatible erythrocyte transfusion showed a marked reduction in lung damage with the lung tissue showing lung morphology similar to that of sham animals (FIGS. 18D-18F). Animals receiving rescue dosing of 40 mg/kg RLS-0071 at 0.5, 60, 90, 120 and 180 after administration of the second hit also displayed lung tissue architecture resembling that of sham animals (FIGS. 18G-18K).

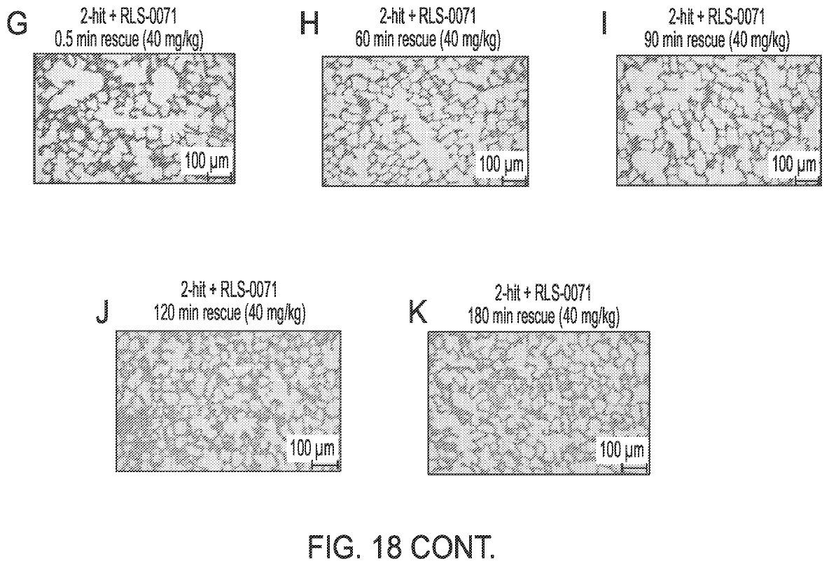

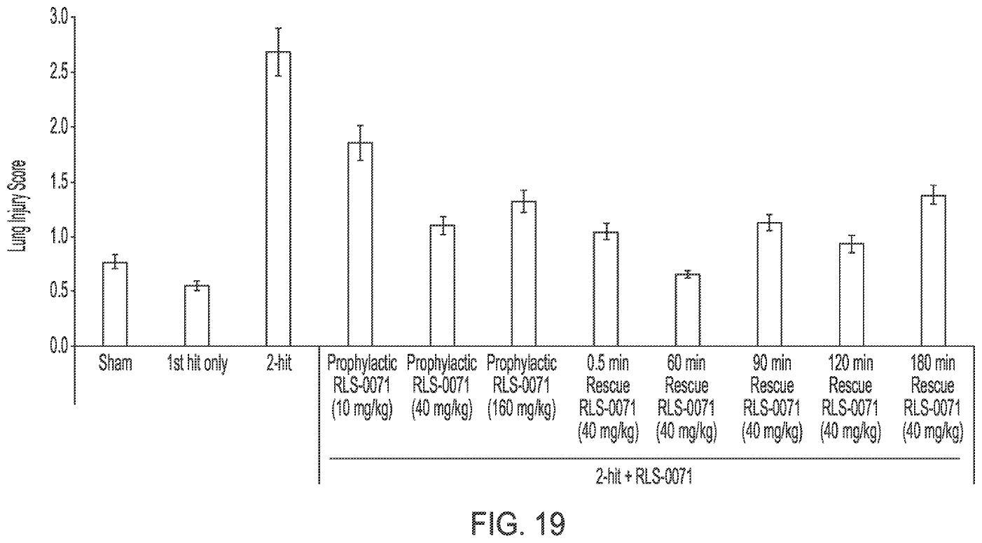

To determine the level of lung tissue protection by RLS-0071 in this model, grading of H&E sections for cell wall thickening for the different treatment groups was performed. Images of randomized microscopy fields were converted to black and white and quantified by ImageJ (NIH) analysis. The ratio of black to white pixels was then determined as a measure of lung damage: as lung damage increased, the alveolar walls thickened, shrinking the alveolar space (white space), resulting in a decrease in white pixels and increase in black pixels. Consistent with the lack of tissue damage directly visualized by microscopic observation of the H&E sections, sham animals and animals receiving the LPS first-hit only had low lung injury scores whereas animals receiving the 2-hit insult demonstrated a much higher injury score as previously demonstrated (FIG. 19). Animals receiving a 10 mg/kg prophylactic dose of RLS-0071 showed significant reduction in lung damage (p=0.002) and this effect was enhanced in animals receiving a 40 mg/kg prophylactic dose (p<0.001) compared to untreated 2-hit animals. Rats prophylactically dosed at 160 mg/kg of RLS-0071 had a similar lung score as the 40 mg/kg dose indicating that dosing beyond 40 mg/kg did not offer any additional protection to the lung tissue (p=0.33 comparing the 40 mg/kg and 160 mg/kg doses) (FIG. 19). To evaluate if dosing animals at various times after the second hit could mitigate lung damage, animals subject to the two-hit insult were treated with 40 mg/kg RLS-0071 at 0.5, 60, 90, 120 and 180 minutes after the erythrocyte transfusion. Treatment with RLS-0071 at all time points after the second hit demonstrated significant reduction in lung damage (all p<0.001) (FIG. 19). These results suggest that a single dose of RLS-0071 can significantly attenuate acute lung injury in this experimental model up to 3 hours after the 2-hit insult.

### RLS-0071 Reduces C5a Production in the Blood

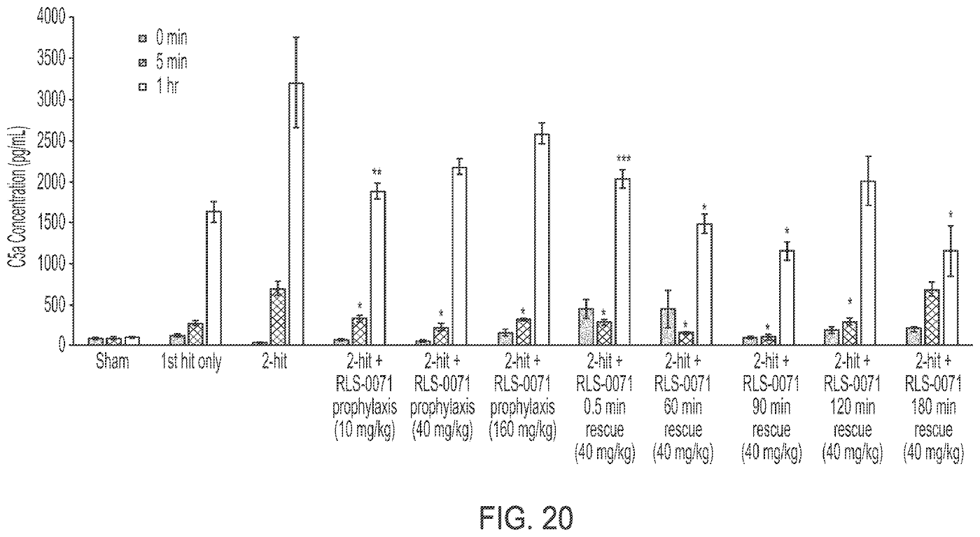

Animals receiving the first-hit of LPS only displayed increased levels of C5a which is attributed to LPS-mediated alternative pathway activation, whereas animals receiving the 2-hit insult demonstrated much higher levels of C5a due to the combination of alternative pathway activation via LPS and classical pathway mediated activation via the incompatible erythrocyte transfusion. To evaluate the effect of RLS-0071 on C5a production in this model, rats subject to the 2-hit insult were treated with prophylactic or rescue doses of RLS-0071 and C5a levels measured from blood samples taken at 0, 5 minutes and 1 hour after the second-hit insult. Sham animals had baseline levels of C5a production whereas animals receiving the LPS first-hit showed increasing levels at 5 minutes and 1 hour (FIG. 20). Animals receiving the 2-hit insult had substantially more C5a production at the 1-hour time point as expected (FIG. 20). Animals receiving RLS-0071 as prophylactic doses of 10, 40 and 160 mg/kg showed significant reduction of C5a at the 5-minute time point for each dose group (p<0.001) and with the exception of the 180-minute rescue dose, reduction of C5a at the 1-hour time points with the 10 mg/kg dose reaching significance (p=0.002) (FIG. 20). As observed with prophylactic dosing, at the 5-minute time point, the 40 mg/kg rescue doses of RLS-0071 administered at 0.5 (p=0.001), 60 (p<0.001), 90 (p<0.001) and 120 (p=0.001) minutes after the 2-hit insult also demonstrated significantly decreased levels of C5a with the exception of animals receiving the rescue dose at 180 minutes (FIG. 20). At the 1-hour time point, all rescue doses had significantly reduced levels of C5a compared to the 2-hit only animals (0.5 (p=0.004), 60 (p<0.001), 90 (p<0.001), 120 (p=0.010) and 180 (p<0.001) minutes. These findings demonstrate that RLS-0071 can significantly inhibit complement activation in this model.

### RLS-0071 Inhibits Free DNA Accumulation in the Blood

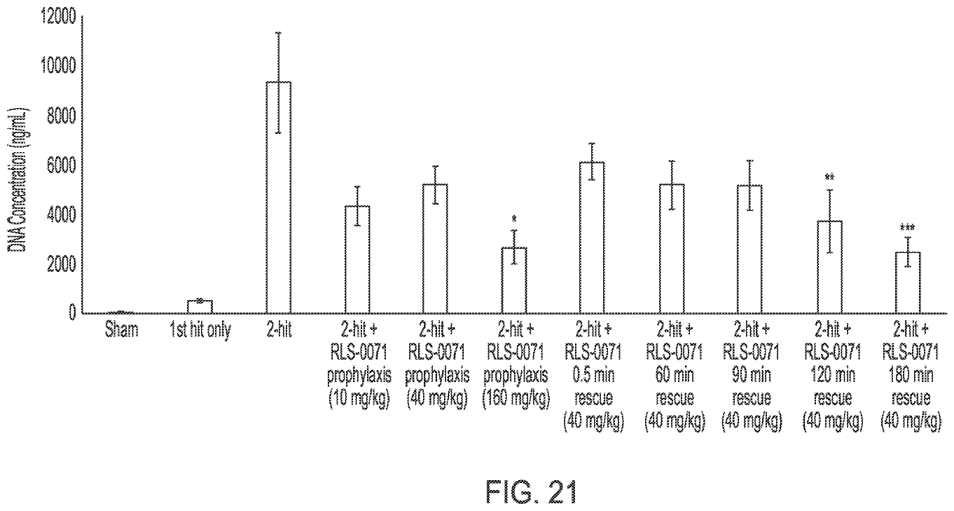

Neutrophil extracellular traps (NETs) released from activated neutrophils have been previously shown to play a pathogenic role in a variety of autoimmune, metabolic, and inflammatory diseases. NETs have been observed in murine models of virally induced ALI as well as TRALI and free DNA in the bloodstream is a biomarker for NETs in the blood of human patients with TRALI as well as COVID-19 patients. To ascertain the effect of RLS-0071 on free DNA levels in the blood, plasma from the different treatment groups were quantified in a PicoGreen assay 4 hours after transfusion. As expected, animals receiving the 2-hit insult showed high plasma levels of free DNA compared to sham animals and animals receiving the first hit of LPS only (FIG. 21). Animals receiving the prophylactic doses of RLS-0071 showed reduced levels of free DNA at the 10 mg/kg, 40 mg/kg and 160 mg/kg doses with the 160 mg/kg dose demonstrating a significant reduction in free DNA compared to 2-hit only animals (p=0.026). Animals subject to rescue doses of 40 mg/kg RLS-0071 after the second hit insult also showed reduced levels of free DNA when dosed up to three hours after the 2-hit injury with the rescue dosing at 120 and 180 minutes reaching statistical significance (p=0.039 and p=0.005, respectively). These results demonstrate that RLS-0071 can modulate NET formation in this disease model and this activity.

### RLS-0071 Reduces Inflammatory Cytokine and Chemokine Levels in the Blood

In severe cases of ALI, alveolar macrophages and epithelial cells may release significant amounts of pro-inflammatory cytokines that exacerbate the disease process leading to acute respiratory disease syndrome (ARDS). This so-called ‘cytokine storm’ has been well documented for virally-induced ALI, in particular the aggressive inflammatory response associated with severe outcomes in COVID-19 [Polidoro R B, Hagan R S, de Santis Santiago R, Schmidt N W. Overview: Systemic Inflammatory Response Derived From Lung Injury Caused by SARS-CoV-2 Infection Explains Severe Outcomes in COVID-19. Front Immunol 2020; 11:1626]. Given the significant ALI seen by lung histology in the inventors' rat 2-hit model, the level of cytokines (FIG. 16A-16C) and chemokines (FIG. 17A-17C) from the blood of rats was measured in absence or presence of RLS-0071 at the terminal 4-hour time point. As expected, plasma from sham animals had low levels of signal for all cytokines tested. Animals receiving the 1-hit of LPS only, had increased levels of cytokines whereas animals receiving the 2-hit insult had greater levels of cytokines which correlate with the increase in lung damage in the 2-hit animals as observed by histology (FIG. 18A-18K). For each of the pro-inflammatory cytokines (IL-1a, IL-1b, IL-6, IFN-g, IL-17, IL-18, TNFa, and RANTES) and chemokines (MCP-1, MIP-1a and MIP-2) evaluated, animals receiving prophylactic dosing of RLS-0071 at 10 or 160 mg/kg and rescue dosing of RLS-0071 at 40 or 160 mg/kg had reduced levels of cytokines and chemokines compared to untreated 2-hit animals with some having significantly reduced levels (FIGS. 16A-16C and 17A-17C). Taken together, these results demonstrate that a single prophylactic or rescue dose of RLS-0071 can mitigate severe ALI in this two-hit model through its dual inhibitory activity of complement inhibition and direct modulation of neutrophil-mediated NET formation.

### Discussion

The objective of this study was to determine if the anti-inflammatory molecule RLS-0071 was able to mitigate ALI in a novel 2-hit rat model that has been described previously [Gregory Rivera M, Hair P S, Cunnion K M, Krishna N K. Peptide Inhibitor of Complement C1 (PIC1) demonstrates antioxidant activity via single electron transport (SET) and hydrogen atom transfer (HAT). PLoS One 2018; 13(3):e0193931]. The LPS first hit followed 30 minutes later with the incompatible erythrocyte second hit results in severe ALI within 4 hours after erythrocyte transfusion. The ALI observed by histology may be mediated by robust neutrophil activation and sequestration into the lung tissue, classical and alternative complement pathway activation and as reported here, significant production of inflammatory cytokines. RLS-0071 is the lead derivative of the PIC1 family of compounds and has been demonstrated to inhibit classical complement activation in in vitro, in vivo and ex vivo studies and inhibit NET formation via inhibition of myeloperoxidase in in vitro and ex vivo studies. Given the dual anti-inflammatory activities of complement inhibition and neutrophil modulation, it was hypothesized that RLS-0071 could inhibit ALI in this animal model. The results herein demonstrate that RLS-0071 delivered as a single dose either prophylactically or as a rescue dose was able to inhibit ALI, even when delivered up to three hours after the second-hit of incompatible erythrocyte transfusion. This was demonstrated by reduced lung damage scores as assessed by histology, reduction of complement activation as measure C5a, decreased levels of free DNA which serves as a biomarker for NETosis and reduction of inflammatory cytokines and chemokines.

ALI ensues following activation of the complement cascade and innate immune response by an external trigger such as a viral infection (e.g., COVID-19, RSV, or influenza) or transfusion and is influenced by the underlying health status of the patient. Complement activation occurs within seconds leading to neutrophil recruitment to the lung tissue and activation of these cells to produce NETs as well as recruit and activate macrophages which in turn produce inflammatory cytokines. This temporal amplification of the immune response leads to a hyperinflammation state that may progress to ALI/ARDS and death. The potent inhibition of ALI observed in this 2-hit model by RLS-0071 may be attributed to the dual anti-inflammatory activities of the molecule, namely complement inhibition and neutrophil modulation at the earliest stage of immune dysregulation. RLS-0071 can inhibit classical complement activity within 30 seconds of IV administration in the rat and can directly modulate neutrophil activation (NETosis and myeloperoxidase activity). By acting within seconds, RLS-0071 can downregulate both the humoral and cellular aspects of the innate immune response at the earliest stage of the inflammatory cascade preventing the cytokine storm and ensuing tissue damage. The ability of RLS-0071 to mitigate ALI in this two-hit model has potential for utility as a clinical therapeutic for virally induced ALI or TRALI.

### Example 2: PA-dPEG24 In Vivo Tissue Binding

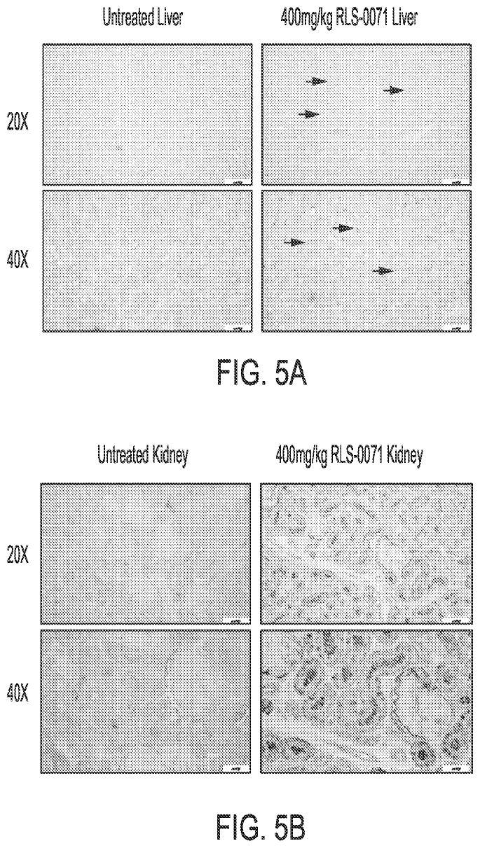

To determine if PA-dPEG24 could be detected in the tissues of rats, male Wistar rats were given a bolus IV dose of 400 mg/kg PA-dPEG24 through an indwelling jugular catheter. Four hours after infusion, rats were sacrificed, and liver and kidneys harvested and fixed in formalin. Tissues from these organs were subsequently sectioned and fixed to glass slides. To determine if PA-dPEG24 was bound to the tissues, the tissue sections were deparaffinized and were probed with an affinity purified rabbit anti-PA-dPEG24 antibody at a 1:1,000 dilution. Antibody signal was then boosted by a combination of biotin and streptavidin peroxidase followed by 3,3′-Diaminobenzidine (DAB) which forms brown precipitate in the presence of the peroxidase. As demonstrated in FIG. 5, microscopic images of liver tissue harvested from rats not receiving PA-dPEG24 showed no staining (left panels) whereas discrete staining was visualized on the tissue from rats treated with PA-dPEG24 (right panels). The same findings were observed for kidney tissue in which animals not receiving PA-dPEG24 demonstrated no staining (left panels), whereas PA-dPEG24-treated animals demonstrated dark staining on the glomerulus and tubules (right panels). These results demonstrated that PA-dPEG24 displays significant and unexpected tissue penetration.

### Example 3: PA-dPEG24 Affects Neutrophil Binding and Adhesion

### PA-dPEG24 Directly Binds Neutrophils In Vitro

The inventors have previously reported that PA-dPEG24 can modulate neutrophils undergoing NETosis in vitro [Hair et al., 2018]. While conducting follow on experiments testing PA-dPEG24 incubated with neutrophils, the inventors noticed that neutrophils exposed to PA-dPEG24 demonstrated decreased numbers adhered to the surface of a glass slide. The inventors then conducted experiments to determine whether PA-dPEG24 was affecting the neutrophils or the surface of the slide was responsible for the reduced adherence. It was determined that the neutrophils could be coated with PA-dPEG24 and remain coated after repeated washing steps, demonstrating that PA-dPEG24 was tightly adhered to the neutrophil surface. Thus, the surface of the slide did not affect neutrophil binding.

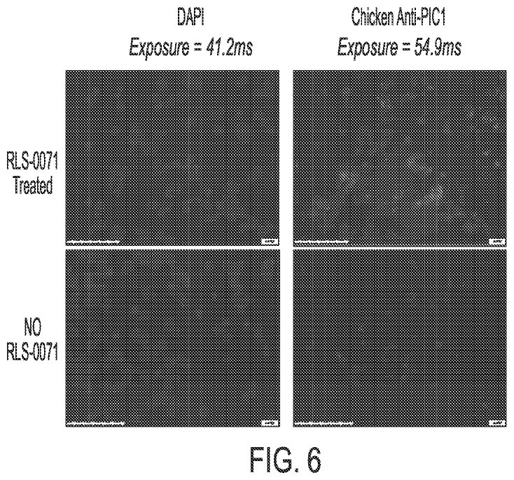

The inhibition of NET formation seen in the in vivo ALI model suggests that PA-dPEG24 directly interacts with neutrophils. To evaluate binding of PA-dPEG24 to neutrophils, purified human neutrophils were cytospun onto glass slides. PA-dPEG24 (1 mM) was then added to one set of slides for 30 minutes and the slides were subsequently washed with PBS. The cells were then incubated with an antibody to PA-dPEG24 (1:1,000 dilution of Chicken Anti-PIC1) followed by a labeled secondary antibody (1:2,000 dilution of Anti-Chicken, Alexa Fluor 488) and counterstained with DAPI. Cells were then visualized by microscopy. In each case, DAPI staining confirmed that the cells were present on the slides and intact. Compared to cells not receiving PA-dPEG24, which showed no signal, neutrophils treated with PA-dPEG24 showed a fluorescence signal demonstrating direct binding of the peptide to the cell surface (FIG. 6).

### PA-dPEG24 Reduces Adhesion of Neutrophils In Vitro

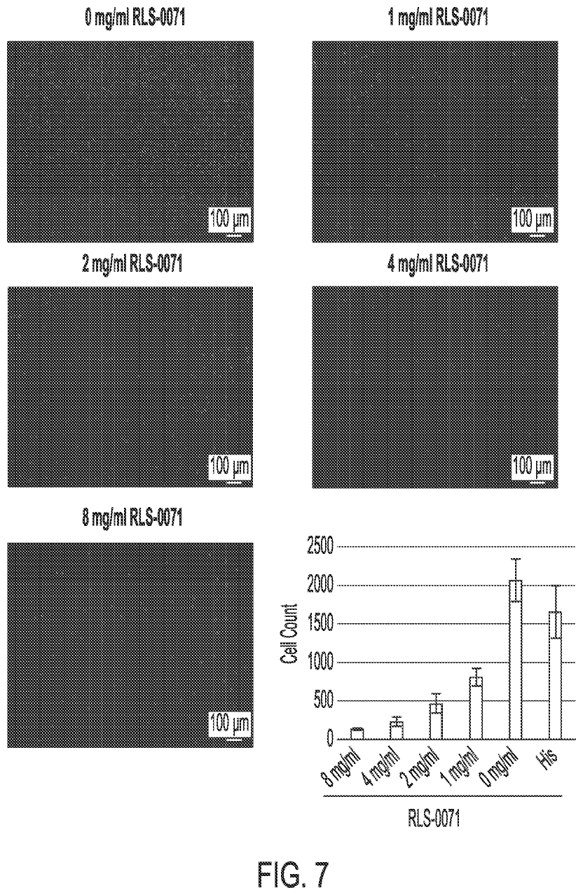

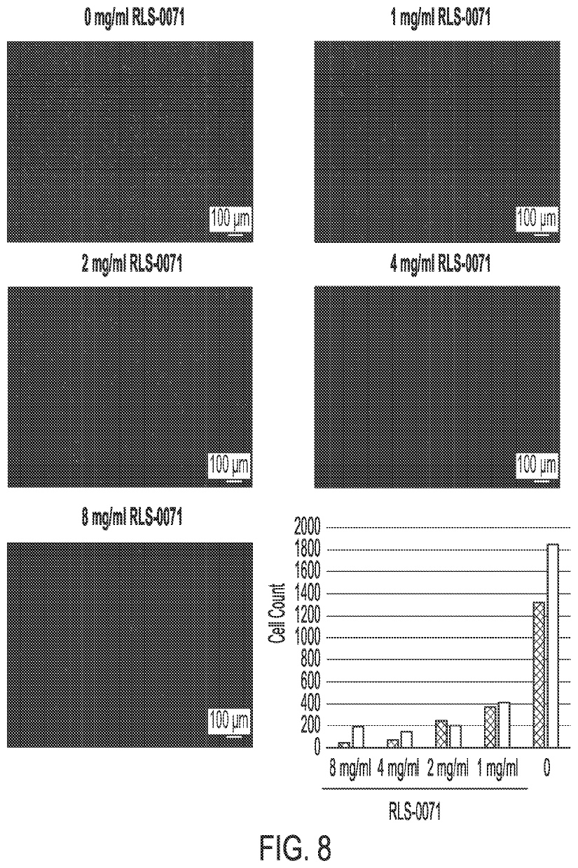

To ascertain whether the binding of PA-dPEG24 has an effect on the ability of neutrophils to adhere to surfaces in vitro, purified human neutrophils were incubated with increasing concentrations of PA-dPEG24, washed twice with PBS, placed on the glass slides, and then incubated in a humidified 37° C. incubator in the presence of 5% C02 for 2.5 hours. Slides were subsequently stained with DAPI (1:1000 in 2% BSA) followed by imaging at 20× by fluorescence microscopy. Quantification of the amount of cell staining was performed using ImageJ analysis (NIH). Increasing amounts of PA-dPEG24 dose-dependently reduced the number of cells adhered to the slides (FIG. 7). To ascertain if adherence of neutrophils was altered on glass slides pre-coated with fibrinogen, purified human neutrophils were incubated with increasing concentrations of PA-dPEG24, washed twice with PBS, placed on the fibrinogen-coated glass slides, and then incubated in a humidified 37° C. incubator in the presence of 5% C02 for 2.5 hours. Slides were subsequently stained with DAPI (1:1000 in 2% BSA) followed by imaging at 20× by fluorescence microscopy. Quantification of the amount of cell staining was performed using ImageJ analysis (NIH). Compared to samples coated on glass slides without fibrinogen treatment, PA-dPEG24 unexpectedly and dose-dependently decreased neutrophil adherence (FIG. 8).

### PA-dPEG24 Increases Human Neutrophil Viability In Vitro

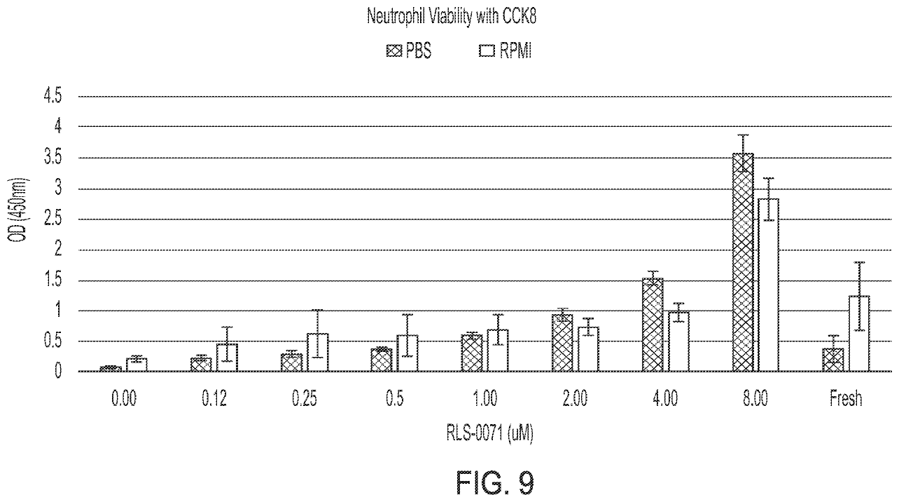

Given the ability of PA-dPEG24 to directly bind human neutrophils and modulate their adhesion to surfaces in vitro, the inventors next determined whether this peptide had a direct effect on cell viability. Cell viability in the presence of increasing amounts of PA-dPEG24 was determined using the Cell Counting Kit-8 (Dojindo). Cell Counting Kit-8 (CCK-8) is a sensitive colorimetric assay for the determination of cell viability in cell proliferation and cytotoxicity assays. The highly water-soluble tetrazolium salt, WST-8, is reduced by dehydrogenase activities in cells to give a yellow-color formazan dye, which is soluble in the tissue culture media. The amount of the formazan dye, generated by the activities of dehydrogenases in cells, is directly proportional to the number of living cells. Neutrophils were isolated from whole blood. Briefly, heparinized whole blood was collected from four different individuals (n=4). The blood was spun on a Hypaque/Ficoll gradient. The pellet was collected, and 3% dextran was added for 20 minutes. The supernatant was collected and washed several times. After a red blood cell lysis, the neutrophils were resuspended in either PBS or RPMI at a concentration of 1.0×10\hat{ }6 cells/mL. ‘Fresh’ neutrophils were taken at this point and 100 uL (100,000 cells/well) were added to a 96-well plate. 10 uL of CCK-8 (Dojindo Molecular Technologies) was added to each well for 2 hours at 37 C. Absorbance at 450 nm was read to determine viability. During this incubation, the remaining cells were incubated with PA-dPEG24 at the various doses for 30 minutes at room temperature. The cells were then washed with 2 mL of PBS/RPMI and resuspended. Cells were incubated another 30 minutes at room temperature and then 100 uL was added to the plate. 10 uL of CCK-8 was added to each well. Cells were incubated with the reagent for 2 hr at 37 C. Absorbance at 450 nm was read to determine viability. Compared to buffer alone, cells treated with PA-dPEG24 in PBS or RPMI showed a dose-dependent increase in viability (FIG. 9), meaning that PA-dPEG24 administration results in an increase in cell viability.

### PA-dPEG24 Binding to Neutrophil and Epithelial Cell Receptors

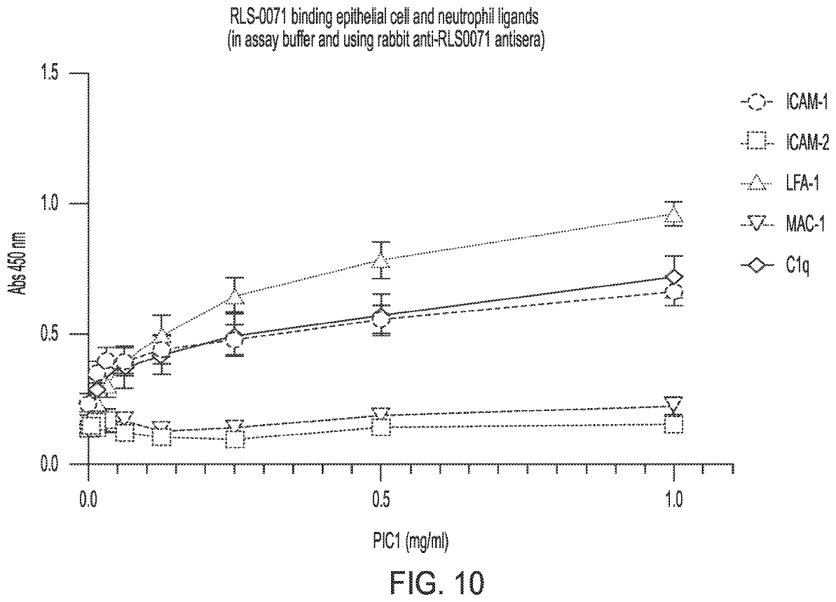

The ability of PA-dPEG24 to bind neutrophils and influence cell viability and adhesion suggests that the peptide specifically binds to neutrophil surface receptors and potentially receptors of endothelial cells required for interactions with neutrophils and adhesion. To address this hypothesis, an ELISA-type binding assay was developed in which neutrophil ligands (LFA-1 and MAC-1/CR3) and epithelial cell ligands (ICAM-1/CD54 and ICAM-2/CD102) were bound to microtiter plates. Plates were then incubated with increasing amounts of PA-dPEG24 in 1% gelatin/PBS buffer probed with antibody to the peptide and developed. PA-dPEG24 bound dose-dependently to the neutrophil receptor LFA-1 but not MAC-1. In addition, PA-dPEG24 bound endothelial receptor ICAM-1 but not ICAM-2. As a positive control, C1q was used as the ligand and bound PA-dPEG24 in a dose-dependent manner as expected (FIG. 10). Additionally, PA-dPEG24 bound to ICAM-3 and ICAM-4 with similar affinity as with ICAM-1 but also demonstrated superior binding to ICAM-5 (FIG. 11).

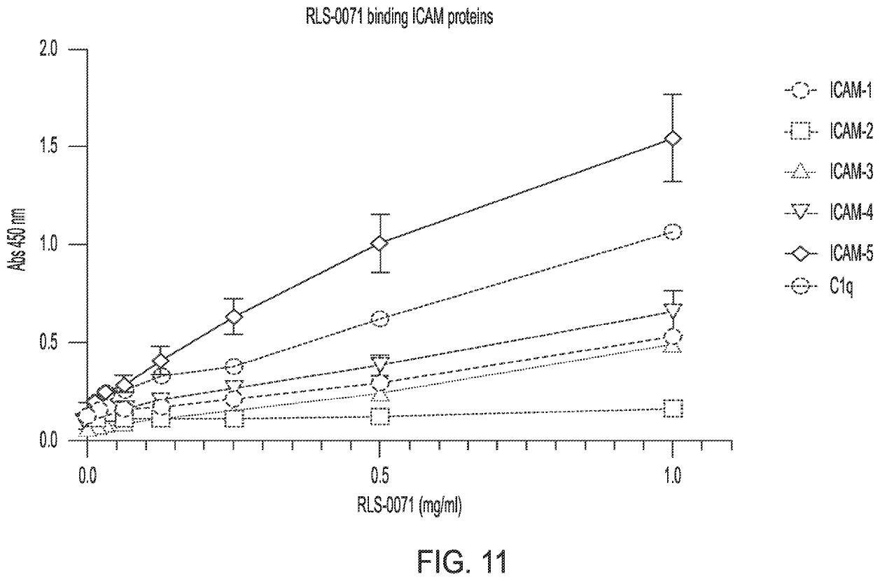

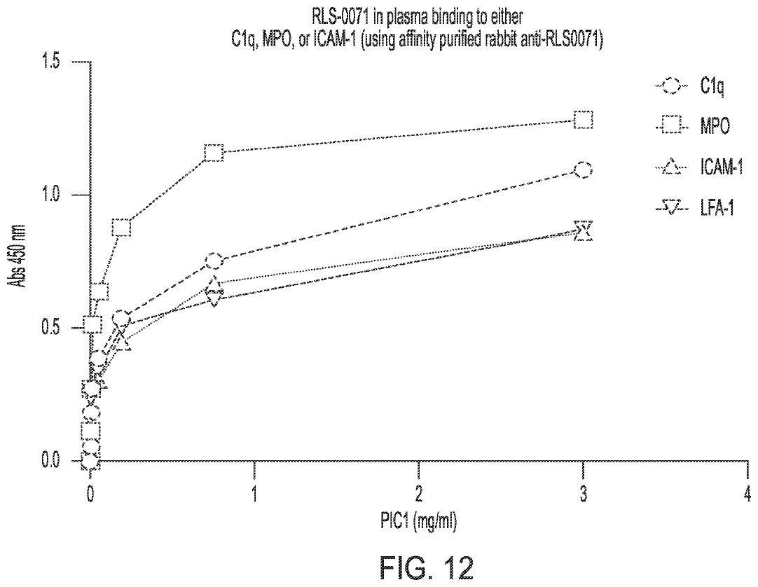

To ascertain if PA-dPEG24 could bind ICAM-1 and LFA-1 in plasma, these receptors were coated onto microtiter plates and then incubated with human plasma containing increasing amounts of PA-dPEG24, probed with antibody to the peptide and then developed. PA-dPEG24 bound dose-dependently to the neutrophil receptor LFA-1 and endothelial receptor ICAM-1. As a positive control, C1q and MPO was used as ligands and bound PA-dPEG24 in a dose-dependent manner as expected (FIG. 12). These results suggest that PA-dPEG24 may modulate neutrophil adherence and viability through specific interactions with cell surface receptors.

The results shown here demonstrate that PA-dPEG24 can directly bind human neutrophils via specific cell surface receptors and modulate neutrophil viability and adherence. In conjunction with the in vivo data demonstrating PA-dPEG24 binding to tissue (liver and kidney) and modulating cytokine levels in rats subject to ALI, these results suggest that PA-dPEG24 can act as a potent anti-inflammatory molecule by directly targeting neutrophils. Additionally, these properties suggest that RLS-0071 may be able to decrease complement mediated inflammation and neutrophil activity in numerous intraocular inflammatory and corneal inflammatory diseases (e.g., uveitis, ROP, and/or retinitis).

### Example 4: Pharmacokinetic Assays of Radiolabeled PA-dPEG24

The pharmacokinetics of [¹⁴C]-PIC1-dPEG24 (radiolabeled PA-dPEG24) related total radioactivity in pooled whole blood and plasma, and metabolite profiles in pooled plasma samples were conducted in male intact Sprague-Dawley rats (N=6) following a single IV dose at 20 or 200 mg/kg PA-dPEG24 (200 μCi/kg, Group 3 and Group 4, respectively). The six rats in Group 3 and Group 4 were further divided into two subgroups for serial blood collections at 10, 30 min and 1, 2, 3, 4, 6, 8, 24, and 48 hr. Blood was pooled by equal volume across animals at each time point, and aliquots (˜200 μL) of pooled blood were removed at each time point for total radioactivity, and the rest of blood samples were centrifuged to obtain plasma. Bile, urine, feces, and cage wash samples were collected up to 72 hr in bile duct cannulated (BDC) rats, and up to 168 hr in intact rats, and terminal blood samples were collected at the end of study (72 hr or 168 hr post-doses). The total radioactivity concentrations in the excreta, blood and plasma were determined by homogenization, combustion and/or liquid scintillation counting (LSC). The metabolite profiles and structure characterization were conducted in pooled plasma, urine, and bile samples using LC-UV/MS as well as radioactive detection. The PK parameters of total radioactivity of [¹⁴C]-PIC1-dPEG24 related components in blood and plasma were obtained by WinNonlin Software.

[¹⁴C]-PIC1-dPEG24 was extensively metabolized in rats and at least 15 metabolites were detected as hydrolyzed and/or dehydrogenated compounds. PIC1-dPEG24 was not stable in solutions, rat urine, and/or rat plasma, and can decompose to M2768 by dehydrogenation. The dehydrogenation position was proposed to be on the two Cys residues to form an internal disulfide. The proposed metabolic pathways showed sequential hydrolyses of peptides from the N-terminal. After sequential loss of Ile, Ala, Leu, Ile, Leu, Glu-Pro, Ile, dehydrogenated Cys-Cys, Gln, Glu, Arg, Ala from M2768, the metabolites M2654, M2583, M2470, M2357, M2244, M2018, M1905, M1701, M1573, M1444, M1288, and M1217 were formed. M160 and M89 were metabolites from M1444 and M1288 after hydrolysis of the amide bond between the amino acids and dPEG24. The metabolic profiles were obtained for rat plasma, urine, and bile, but not for feces due to the low radioactivity. No significant differences were observed for the metabolism of PIC1-dPEG24 between the low and high dose groups.

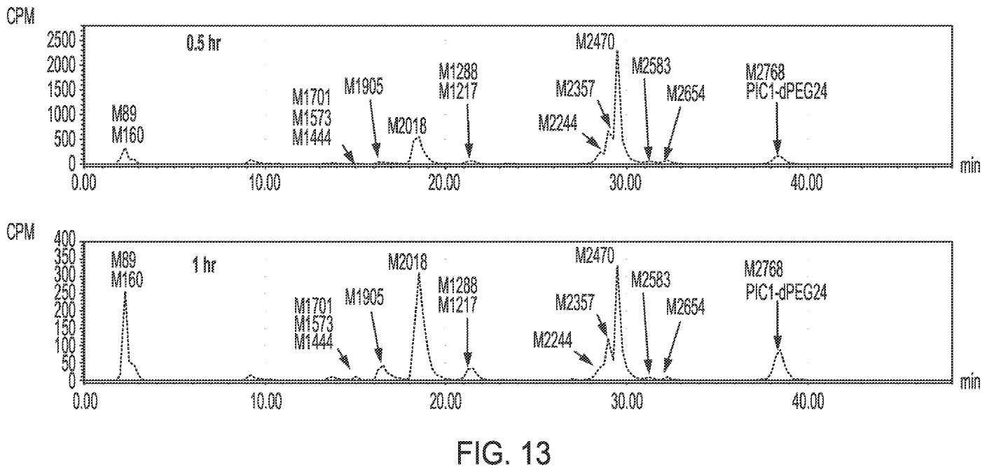

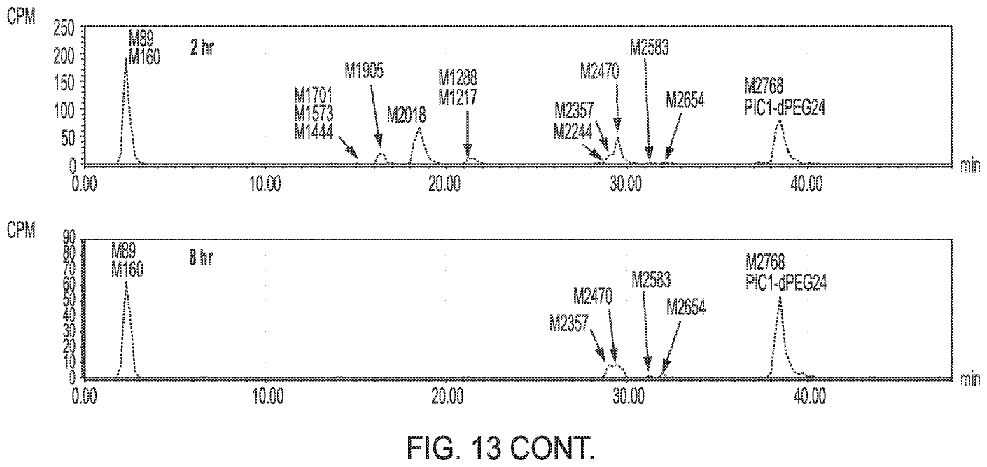

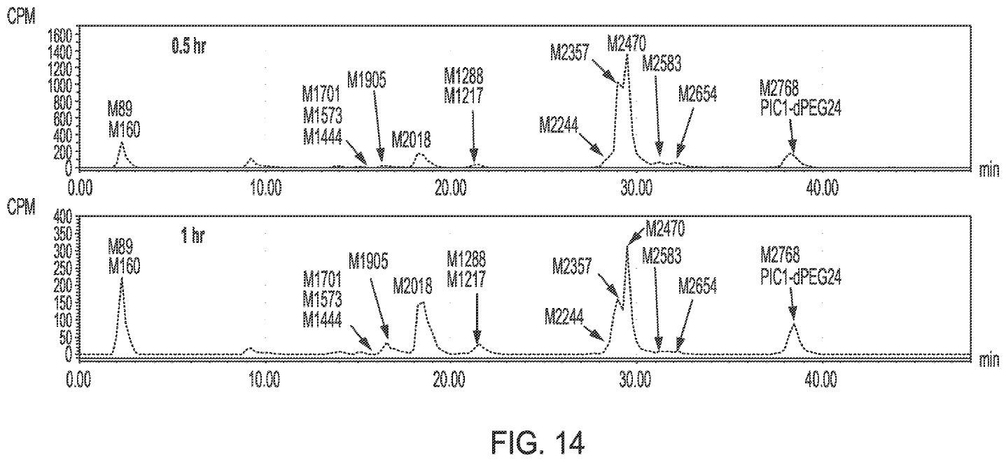

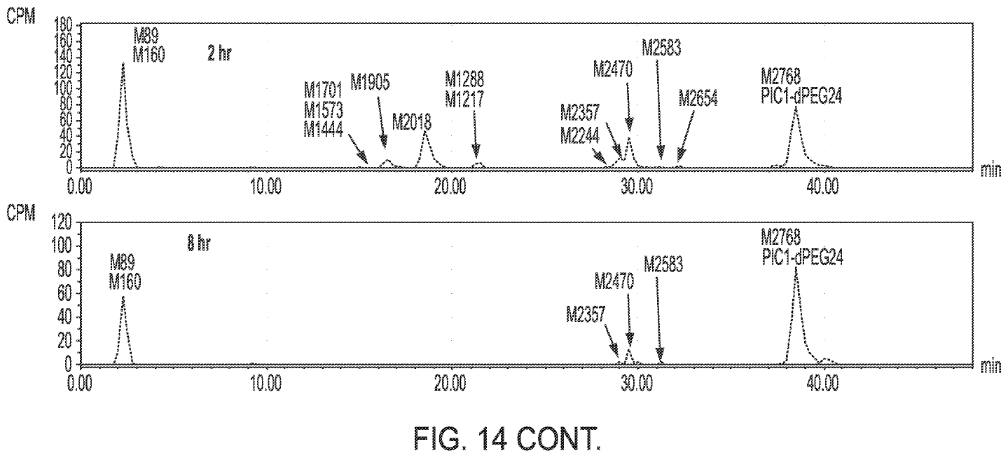

The radioactive profiles of rat plasma for the low dose group (Group 3) and the high dose group (Group 4) were qualitatively similar. In the 0-24 hr AUC pooled samples, parent PIC1-dPEG24 and its dehydrogenated product M2768 together represented about 12% and 16% of the plasma radioactivity in the low dose and high dose groups, respectively. Metabolites M89/M160, M2018, M2244/M2357/M2470, M2583, and M2654 were detected at relatively higher amounts and represented about 7%, 12%, 52%, 4%, and 10%, respectively, of the plasma radioactivity in the low dose group, and 9%, 7%, 44%, 7%, and 12%, respectively, of the plasma radioactivity in the high dose group. Other metabolites were minor, and each represented less than 3% of the plasma radioactivity. The estimated concentrations of each radioactive peak in AUC_{0-24 hr} pooled plasma represented the mean concentration within 0-24 hr. In the low dose group, the parent PIC1-dPEG24 and its dehydrogenated product M2768 together was calculated as 187 ng Eq/g. Metabolites M89/M160, M2018, M2244/M2357/M2470, M2583, and M2654 had estimated concentrations of 114, 183, 252, 65, and 161 ng Eq/g, respectively. In the high dose group, the parent PIC1-dPEG24 and its dehydrogenated product M2768 together was calculated as 2225 ng Eq/g. The calculated concentrations of metabolites, M89/M160, M2018, M2244/M2357/M2470, M2583, and M2654 were 1237, 981, 3024, 953, and 1678 ng Eq/g, respectively. In the time point pooled samples up to 8 hours, the percent of parent, PIC1-dPEG24, and its dehydrogenated product M2768 together increased over time from about 5% to 40% in the low dose group, and from 7% to 60% in the high dose group, which might indicate the metabolites of PIC1-dPEG24 eliminated faster than PIC1-dPEG24 at the low and high doses (see Table 1 and FIGS. 13 and 14). The major metabolites (>10% of plasma radioactivity) were observed as M89/M160, M2357, M2470, and M2018, and their calculated concentrations decreased over time from 0.5 hr to 8 hr.

In conclusion, the metabolism, pharmacokinetics, and excretory mass balance of [¹⁴C]-PIC1-dPEG24 were studied in male intact or BDC rats following a single IV dose at 20 or 200 mg/kg of [¹⁴C]-PIC1-dPEG24. Dosed radioactivity was excreted rapidly and the majority of the dose (>70% of the dose) recovered within 24 hr post-dose and mainly via urine and only small to trace amounts of the dose were found in feces and/or bile, while about 82% to 91% of the dose were found in excreta up to 168 hr. The results of this study indicated that urinary excretion was major route of elimination of [¹⁴C]-PIC1-dPEG24-related radioactivity in male BDC and intact rats following a single IV dose at 20 and 200 mg/kg. Under the same IV doses in intact rats, the plasma PK parameters were characterized as long elimination half-lives (≥40 hr) for total radioactivity. [¹⁴C]-PIC1-dPEG24 was quickly hydrolyzed to multiple hydrolyzed/dehydrogenated metabolites in male BDC rats. M89/M160, M2018, M2244/M2357/M2470 were the major metabolites observed in plasma, while unchanged parent compound and its dehydrogenated product M2768 together were observed as a small radioactive peak at 30 min post-IV-injection in pooled plasma, but it was still detectable at 8 hr time point. The major metabolites detected in urine were M1444/M1701/M1573, M2018, M1288, M1217, M2244/M2357, and M2470, and unchanged parent compound was not detectable at the 20 and 200 mg/kg dose. Hydrolysis and dehydrogenation were the major metabolic pathways of [¹⁴C]-PIC1-dPEG24 in rats following a single IV dose. Significant differences were not observed for the metabolism, pharmacokinetics, and excretion of [¹⁴C]-PIC1-dPEG24 between the IV doses of 20 or 200 mg/kg in male rats.

### Stability of PIC1-dPEG24 in Solution, Rat Plasma and Rat Urine

A dehydrogenated product M2768 was detected in the diluted dose solution in water after 6-day storage at −20° C. freezer. The data indicated that PIC1-dPEG24 was not stable. To explore the stability of PIC1-dPEG24 in rat plasma and urine, [¹⁴C]-PIC1-dPEG24 was spiked into control blank rat plasma and a pre-dose urine samples. The amounts of the dehydrogenated product M2768 increased significantly in both rat plasma and urine spiked samples. These data indicated PIC1-dPEG24 was either not stable in rat plasma/urine, or could decompose to a dehydrogenated product during sample processing. Therefore, based on these data, the integration of M2768 and PIC1-dPEG24 were combined for rat plasma profiles since M2768 could be formed without enzymatic involvement. In addition, the other dehydrogenated metabolites including M1905, M2018, M2244, M2357, M2470, M2583, and M2654 could be formed from M2768 after hydrolysis or formed by hydrolysis first and then decomposed to corresponding dehydrogenated metabolites. The dehydrogenation was proposed to be a disulfide formation at the Cys-Cys dipeptide residues.

### Metabolite Profiles of Pooled Plasma

Radiochromatograms of pooled plasma samples (0-24 hr AUC pool) and the time point pools at 0.5, 1, 2, and 8 hr from the low and high dose groups are shown in FIGS. 13 and 14. Percent distribution expressed as percent of radioactive peak are shown in Table 1. A total of 15 metabolites were observed in rat plasma. The radioactive profiles of rat plasma samples for the low dose group and the high dose group were qualitatively similar.

TABLE 1
Peak Distribution of PIC1-dPEG24 & Metabolites in Pooled Plasma Samples of Male
Rats Following a Single 20 or 200 mg/kg IV Dose of [¹⁴C]-PIC1-dPEG24
Adjusted Peak Distribution (ROI %) in Rat Plasma

| | | Retention | | | | | | | | | | |
|---|---|---|---|---|---|---|---|---|---|---|---|---|
| Peak | | Time | Group 3 | | | | | Group 4 | | | | |
| # | Name | (min) | 0.5 hr | 1 hr | 2 hr | 8 hr | AUC_{0-24 hr} | 0.5 hr | 1 hr | 2 hr | 8 hr | AUC_{0-24 hr} |
| 1 | M89 | 2.4 | 7.55 | 15.63 | 36.02 | 45.73 | 7.31 | 7.77 | 17.30 | 36.42 | 34.14 | 9.07 |
| 2 | M160 | 2.4, 2.9 | | | | | | | | | | |
| 3 | Unknown | 9.1 | 2.15 | 1.27 | 0.23 | X | 1.46 | 3.80 | 1.77 | 0.15 | 0.35 | 2.92 |
| 4 | M1444 | 14.6 | 0.45 | 0.86 | 0.23 | X | X | 0.44 | 0.60 | 0.30 | X | X |
| 5 | M1701 | 14.8 | | | | X | X | | | | X | X |
| 6 | M1573 | 15.0 | | | | X | X | | | | X | X |
| 7 | M1905 | 16.1 | 1.43 | 5.03 | 4.55 | X | 0.59 | 0.94 | 3.54 | 3.64 | X | 0.52 |
| 8 | M2018 | 17.9 | 18.91 | 32.12 | 19.32 | X | 11.80 | 7.79 | 22.17 | 16.54 | X | 7.19 |
| 9 | M1288 | 20.9, 30.5 | 2.00 | 4.05 | 3.18 | X | 0.68 | 1.44 | 3.54 | 1.82 | X | 0.31 |
| 10 | M1217 | 21.4, 30.6 | | | | | | | | | | |
| 11 | M2244 | 28.5 | 4.36 | 2.50 | 0.45 | X | 51.59 | 3.27 | 13.72 | 3.03 | X | 44.40 |
| 12 | M2357 | 29.1 | 10.40 | 6.79 | 2.16 | 3.88 | | 17.42 | | | 0.70 | |
| 13 | M2470 | 29.5 | 43.75 | 21.93 | 10.11 | 8.14 | | 44.01 | 26.22 | 9.56 | 4.57 | |
| 14 | M2583 | 31.2 | 1.65 | 0.78 | 0.68 | 0.39 | 4.19 | 3.09 | 1.25 | 0.46 | 0.70 | 6.98 |
| 15 | M2654 | 31.9 | 2.16 | 0.82 | 0.68 | 1.55 | 10.34 | 2.82 | 0.78 | 0.61 | X | 12.30 |
| 16 | M2768 | 37.1 | 5.18 | 8.24 | 22.39 | 40.32 | 12.04 | 7.20 | 9.12 | 27.47 | 59.53 | 16.31 |
| 17 | PIC1-dPEG24 | 38.2 | | | | | | | | | | |

| | | | | | | | | | | |
|---|---|---|---|---|---|---|---|---|---|---|
| Identified Total (%) | 97.85 | 98.73 | 99.77 | 100.00 | 98.54 | 96.20 | 98.23 | 99.85 | 99.65 | 97.08 |

The radioactive profiles of rat plasma for the low dose group (Group 3) and the high dose group (Group 4) were qualitatively similar. In the 0-24 hr AUC pooled samples, parent PIC1-dPEG24 and its dehydrogenated product M2768 together represented about 12% and 16% of the plasma radioactivity in the low dose and high dose groups, respectively. Metabolites M89/M160, M2018, M2244/M2357/M2470, M2583, and M2654 were detected at relatively higher amounts and represented about 7%, 12%, 52%, 4%, and 10%, respectively, of the plasma radioactivity in the low dose group, and 9%, 7%, 44%, 7%, and 12%, respectively, of the plasma radioactivity in the high dose group. Other metabolites were minor, and each represented less than 3% of the plasma radioactivity. The estimated concentrations of each radioactive peaks in AUC_{0-24 hr} pooled plasma represented the mean concentration within 0-24 hr. In the low dose group, the parent PIC1-dPEG24 and its dehydrogenated product M2768 together was calculated as 187 ng Eq/g. Metabolites M89/M160, M2018, M2244/M2357/M2470, M2583, and M2654 had estimated concentrations of 114, 183, 252, 65, and 161 ng Eq/g, respectively. In the high dose group, the parent PIC1-dPEG24 and its dehydrogenated product M2768 together was calculated as 2225 ng Eq/g. The calculated concentrations of metabolites, M89/M160, M2018, M2244/M2357/M2470, M2583, and M2654 were 1237, 981, 3024, 953, and 1678 ng Eq/g, respectively. In the time point pooled samples up to 8 hours, the percent of parent PIC1-dPEG24 and its dehydrogenated product M2768 together increased over time from about 5% to 40% in the low dose group, and from 7% to 60% in the high dose group, which indicated the metabolites of PIC1-dPEG24 eliminated faster than PIC1-dPEG24 at the low and high doses. The major metabolites (>10% of plasma radioactivity) were observed as M89/M160, M2357, M2470, and M2018, and their calculated concentrations decreased over time from 0.5 hr to 8 hr.

### Discussion

The PK modeling studies in rats, dogs, and monkeys (separate studies) demonstrate that PA-DPEG24 is rapidly cleared from the blood stream and the material that is not excreted is sequestered in another compartment (tissue bed). Over time the peptide slowly released back into the circulation. This is reflected in the PK profile which shows a very long tail of low-level peptide in circulation.

In the time point pooled samples up to 8 hours, the percent of parent, PIC1-dPEG24, and its dehydrogenated product M2768 together increased over time from about 5% to 40% in the low dose group, and from 7% to 60% in the high dose group. That is to say, the PIC-dPEG24 intact molecule was initially detected as a small radioactive peak at 30 min post-IV-injection in pooled plasma, was still detectable at 8 hr time point. This surprising result shows that a portion of the dosed molecule is sequestered out of the central vasculature in tissue beds where it is protected from degradation and then released back into the bloodstream intact. This is a novel and completely unexpected finding given that peptides are notoriously unstable in the bloodstream.

### Example 5: PA-dPEG24 does not Interfere with C1q-Antibody Complexes Binding to C1q-Receptors on Monocyte Cells

C1q is the first complement component of the classical pathway of complement. C1q along with the serine protein tetramer C1s-C1r-C1r-C1s is known as the C1 complex. Upon binding of the globular heads of C1q by antibody-coated pathogens, C1q undergoes a conformational change that allows activation of the C1s-C1r-C1r-C1s tetramer which is located in a hydrophobic pocket of the C1q collagen-like domain. Activated C1s-C1r-C1r-C1s then cleaves C4, followed by C2 to cause amplification of the classical complement pathway resulting in effector functions such as C3a and C5a generation, C3b opsonization and membrane attack complex formation (Cooper, 1985).

In the bloodstream, circulating C1 complex and free C1q are both present. Along with activation of the classical pathway, C1q also plays a critical homeostatic role in the clearing of cell debris such as apoptotic bodies and immune complexes. This clearing occurs via the globular heads of C1q binding the apoptotic or immune complex cargo and then engaging C1q receptors on phagocytes (i.e., neutrophils and monocytes/macrophages) that recognize the collagen-like region of C1q. These complexes are ultimately phagocytosed. This process prevents accumulation of apoptotic debris/immune complexes and development of autoimmunity (e.g., systemic lupus erythematosus).

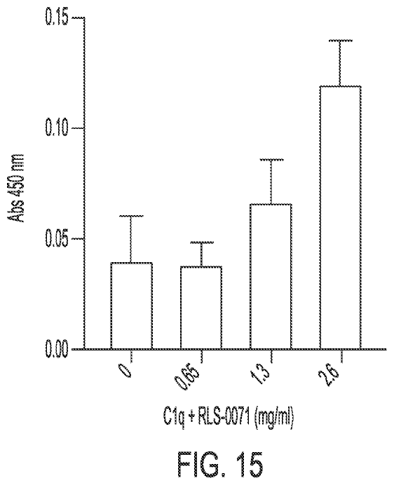

PA-dPEG24 has been demonstrated to bind to the hydrophobic pocket of the collagen-like region and not the globular heads of the C1q molecule (Sharp et al., 2015). To verify that PA-dPEG24 does not interfere with the interaction of C1q with C1q receptors on phagocytes, the following experiment was conducted. Freshly purified human monocytes were allowed to adhere to a 96 well tissue culture plates and nonadherent lymphocytes were removed. C1q alone or in the presence of increasing concentrations of PA-dPEG24 was then added to the wells and allowed to incubate. Unbound C1q was washed off and ovalbumin rabbit immune complexes were added and allowed to incubate. Unbound immune complexes were washed off and an anti-rabbit HRP antibody was used to detect bound immune complexes, followed by development with TMB and quenching of the reaction with 1N H₂SO₄ and detection at 450 nm in a plate reader. Separately, an anti-C1q antibody was used after the C1q incubation to confirm binding of C1q to the monocytes. The presence of increasing amounts of PA-dPEG24 did not reduce the level of C1q-immune complexes. Surprisingly, the amount of C1q detected increased with increasing amounts of PA-dPEG24 (FIG. 15). These results suggested that PA-dPEG24 does not interfere with the binding of C1q-immune complexes to its cognate receptors on monocytes and thus would not be predicted to interfere with C1q's homeostatic functions (i.e., clearance of immune complexes/apoptotic debris). Indeed, PA-dPEG24 was surprisingly shown to increase C1q binding to monocytes. This finding suggests that PA-dPEG24 may be able to increase C1q-mediated clearance of immune complexes in vivo. Therefore, without wishing to be bound by theory, this increase has implications for diseases where immune complexes contribute to pathogenesis, including numerous inflammatory ophthalmologic diseases (e.g., uveitis or retinitis), which are situations were increased rapidity of immune complexes can potentially lessen disease severity.

### Example 5: Safety and Pharmacokinetic Profile of PA-dPEG24 Delivered Via Intravitreal (IVT) Injection

### Methods

Safety study. A maximum deliverable dose of RLS-0071 (160 mg/ml in 5 microliters total volume) was delivered intravitreally to the right eye of 4 male Wistar rats. A saline control was administered to the left eye. For this procedure, animals were anesthetized with isoflurane and also received the topical anesthetic proparacaine. Additionally, animals received the topical antibiotic tobramycin after injection. Slit lamp examinations were performed at the indicated time points up to 72 hours post-injection and pathology graded using a modified MacDonald-Shadduck Ocular Grading system with the following scoring scale: 0, no pathology; 1, slight pathology; 2, moderate pathology; 3/4, severe pathology.

Pharmacokinetic study. A maximal deliverable dose of RLS-0071 (160 mg/ml, 5 μl total volume) was administered intravitreally to the right and left eye of male Wistar rats. Animals were euthanized at 5 min (n=4 rats), 1 hour (n=3 rats), 4 hours (n=2 rats), 24 hours (n=2 rats), 4 days (n=4 rats) and 10 days (n=3 rats) by C02 asphyxiation. Eyes were enucleated at the time of euthanasia and immediately stored in −80° C. conditions. Twenty-four hours later, frozen eyes were sectioned into anatomical compartments and stored again in −80° C. conditions for future processing.

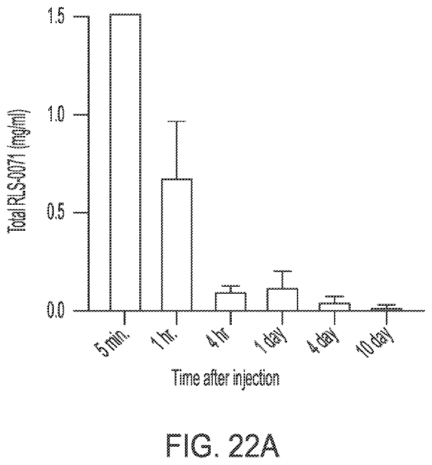

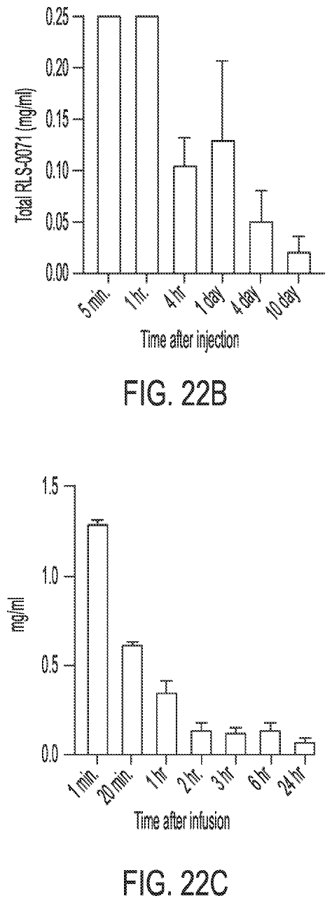

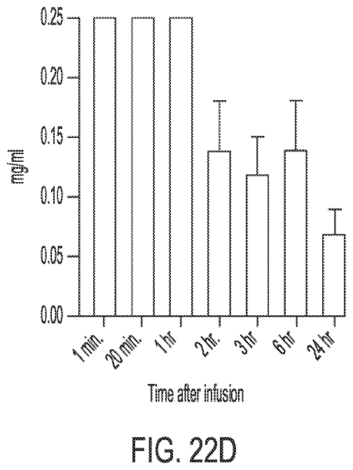

RLS-0071 sandwich ELISA. For determination of RLS-0071 half-life, volumes of the vitreous fluid for each sample were estimated and recorded based on the meniscus of the sample in the microfuge tube (compared to standard known quantity), as the samples were viscous and could not be easily drawn into a pipet. Next, 100 ul of 1% BSA/PBS was added to each sample and they were placed in a shaker overnight at 4° C. The next day, the samples were spun at 5,000 rpm for 5 minutes and the supernatant was collected and applied to the RLS-0071 sandwich ELISA which utilized a bound primary chicken polyclonal antibody to RLS-0071 to capture the peptide and a primary rabbit polyclonal antibody against RLS-0071 to detect any peptide bound to the plate. The rabbit antibody was then probed with goat anti-rabbit secondary antibody conjugated to HRP, developed with TMB and the plate read at 450 nm by spectrophotometry. Data shown in FIG. 22A-22D are the means from four independent experiments. Error bars denote standard errors of the means (SEM).

DAB staining for RLS-0071 in ocular tissue. To determine tissue distribution of RLS-0071 in the retina, animals receiving intravitreally administered saline (control) or RLS-0071 as described above were euthanized 5 minutes post-IVT for saline animals and 1-hour post-IVT for animals receiving RLS-0071. The eyes were then harvested, and ocular tissues isolated and processed for histology and staining with DAB using primary rabbit polyclonal antibody to RLS-0071.

### Results

Intravitreal injection of RLS-0071 is safe. Rats were intravitreally injected with 160 mg/kg (maximal deliverable dose) of RLS-0071 and eyes examined for pathology by slit lamp at the following time points: Pre-JVT, 0.5, 2, 24, 48 and 72 hours. Pathology was determined using a modified MacDonald-Shadduck Ocular Grading system with a score of 0 indicating no pathology and 3/4 indicating severe pathology. No RLS-0071 related toxicity was observed for all 4 animals similar to saline controls (Table 2). These results demonstrate the RLS-0071 can be safely delivered to the vitreous of the rat eye with no adverse effects out to 3 days.

TABLE 2
Safety assessment of IVT dosing of RLS-0071 in rats.

| | | Pre-IVT | | 30 min | | 2 hour | | 24 hour | | 48 hour | | 72 hour | |
|---|---|---|---|---|---|---|---|---|---|---|---|---|---|
| | Rat | | RLS- | | RLS- | | RLS- | | RLS- | | RLS- | | RLS- |
| | # | Saline | 0071 | Saline | 0071 | Saline | 0071 | Saline | 0071 | Saline | 0071 | Saline | 0071 |
| Conjunc- | 1 | 0 | 0 | 0 | 0 | 0 | 0 | 0 | 0 | 0 | 0 | 0 | 0 |
| tiva | 2 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 |
| Anterior | 1 | 0 | 0 | 0 | 0 | 0 | 0 | 0 | 0 | 0 | 0 | 0 | 0 |
| Chamber | 2 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 |
| Iris | 1 | 0 | 0 | 0 | 0 | 0 | 0 | 0 | 0 | 0 | 0 | 0 | 0 |
| | 2 | 0 | 0 | 0 | 0 | 0 | 0 | 0 | 0 | 0 | 0 | 0 | 0 |
| | 3 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 |
| Cornea | 1 | 0 | 0 | 0 | 0 | 0 | 0 | 0 | 0 | 0 | 0 | 0 | 0 |
| | 2* | 0 | 0 | 1 | 0 | 1 | 0 | 1 | 0 | 1 | 0 | 0 | 0 |
| | 3 | 0 | 0 | 0 | 0 | 0 | 0 | 0 | 0 | 0 | 0 | 0 | 0 |
| | 4 | 0 | 0 | 0 | 0 | 0 | 0 | 0 | 0 | 0 | 0 | 0 | 0 |

Intravitreally delivered RLS-0071 has an extended half-life. To assess the pharmacokinetics of IVT delivered RLS-0071, rat eyes were injected IVT with 160 mg/ml of RLS-0071 in a total volume of 5 microliters. At 5 minutes, 1 hour, 4 hours, 1 days, 4 days and 10 days after injection, eyes from the animals were removed at euthanasia and the vitreous fluid processed for analysis to detect RLS-0071 in a sandwich ELISA. Surprisingly, RLS-0071 could be detected up to 10 days post-IVT injection and was detected at 0.12 mg/ml at 24 hours (FIG. 22A-22B). In comparison, rats infused IV with 200 mg/ml (800 mg/kg) RLS-0071 showed a level of 0.07 mg/ml at 24 hours and was undetectable thereafter (FIG. 22C-22D). These data demonstrated that IVT delivered RLS-0071 unexpectedly has a much longer half-life in the eye than peptide delivered intravenously.

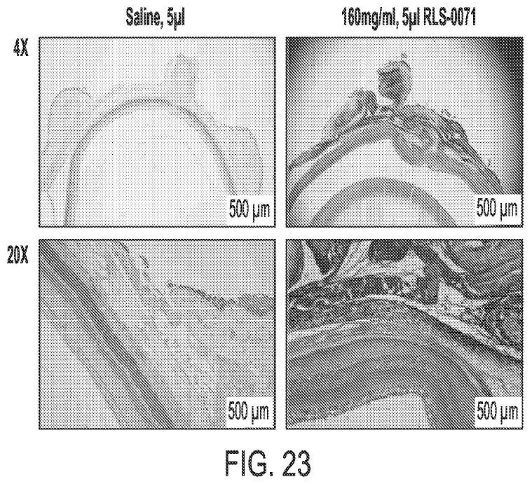

Intravitreally delivered RLS-0071 robustly stains retinal tissue. Rat absorption, distribution, metabolism, and excretion (ADME) studies have previously demonstrated that radiolabeled RLS-0071 has significant tissue distribution in various tissue beds when delivered IV. Additionally, RLS-0071 has been shown to bind ICAM1, 3, 4 and 5 in a plate binding assay; these adhesion molecules are present to varying degrees on endothelial and epithelial cells, suggesting RLS-0071 may bind to retinal tissue. To assess if RLS-0071 bound to retinal tissue in rats receiving IVT injection of the peptide, the retinal tissue was processed for histology and incubated with the polyclonal rabbit anti-RLS-0071 antibody followed by DAB staining. Compared to rat eyes injected IVT with saline, eyes receiving an IVT injection of RLS-0071 showed significant DAB signal 1 hour after injection (FIG. 23). The robust staining of all the tissue levels of the eye was unexpected, because the layers of the eye have barriers to compartmentalize and block infectious particles and other non-nutritive molecules from crossing from one layer to the next. Collectively, these findings demonstrate that IVT administered RLS-0071 has no adverse effects on the eye of the rat and shows prolonged half-life and tissue penetration of the retina. Without wishing to be bound by theory, it is suggested that RLS-0071 may have therapeutic benefit in inhibition of acute diseases of the eye where complement and neutrophil-mediated inflammation plays a pathogenic role.

### Example 7: RLS-0071 Inhibition of Complement Activation in Blood Versus Tissues at Low Dose in a 2-Hit Rat Acute Lung Injury (ALI) Model

### Background and Results

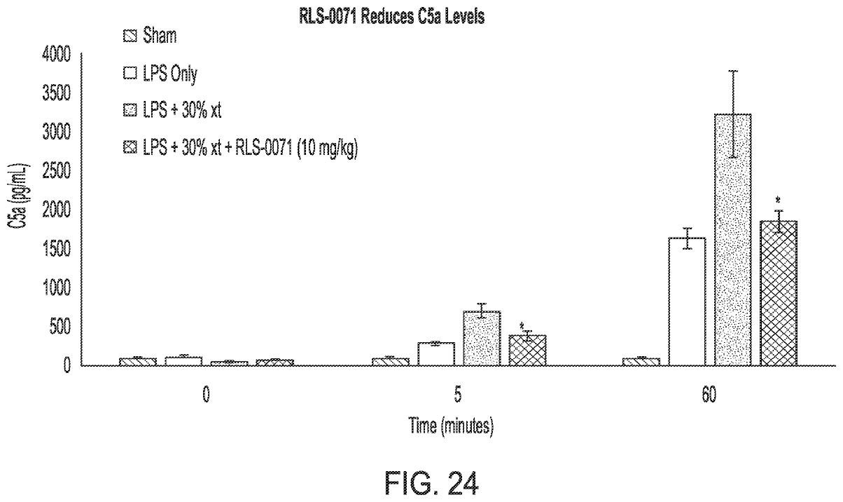

RLS-0071 was tested in a two-hit rat model of ALI. The first insult is neutrophil stimulation with lipopolysaccharide (LPS) followed 30 minutes later by a second insult of classical complement activation with incompatible erythrocytes. This model can produce dramatic neutrophil infiltration into alveolar walls, thickening the walls and reducing alveolar airspace by 85%. As shown herein, RLS-0071 given as a single dose IV at 10 mg/kg up to 160 mg/kg produced similar protection from lung damage. NET generation was measured by free DNA quantitation in plasma and showed that 10 mg/kg yielded similar reduction compared with 160 mg/kg. Reduced pro-inflammatory cytokine production (IL-1, IL-6, IL-17 and TNFα) was demonstrated in animals treated with RLS-0071. Complement inhibition was demonstrable by measurement of C5a in rat plasma for 10 mg/kg RLS-0071 at 5 and 60 minutes after the second hit (FIG. 24). Given the short 5-minute half-life of C5a, the measurement of elevated C5a at 60 minutes is consistent with tissue generated C5a. These data demonstrated that RLS-0071 can inhibit activation of complement in peripheral tissues at low doses, e.g., 10 mg/kg IV.

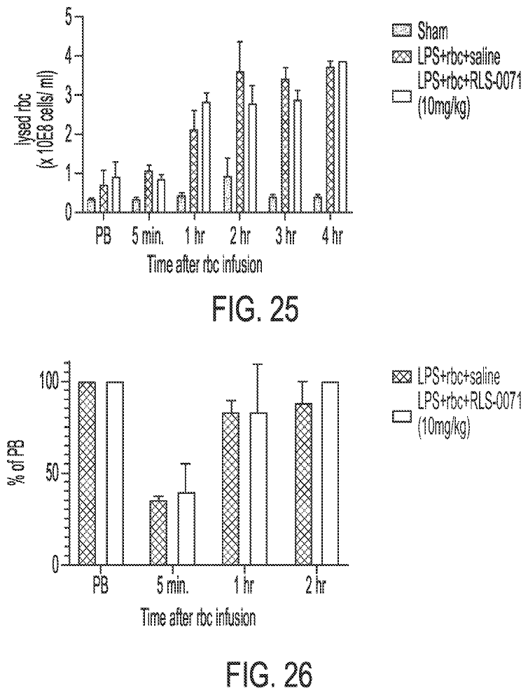

Complement inhibition in the bloodstream of rats in the 2-hit ALI model was measured by two different methods. The first method measured free hemoglobin in the plasma of the rats over time, by measuring intravascular hemolysis of the transfused incompatible erythrocytes. As seen in FIG. 25, rats receiving the incompatible transfusion showed increased intravascular hemolysis over time reaching a near maximal level by 1 hour. RLS-0071 given at 10 mg/kg IV demonstrated no inhibition of classical complement pathway mediated hemolysis compared with saline treatment (FIG. 25). The plasma samples were also analyzed by mCH50 ex vivo, and showed a transient decrease in mCH50 at 5 minutes due to complement component consumption resulting from classical pathway activation by the incompatible transfusion with a rebound to nearly normal mCH50 values at 1 hour (FIG. 26). RLS-0071 at 10 mg/kg IV did not inhibit mCH50 compared with saline treated control (FIG. 26). These two assays demonstrate that RLS-0071 at 10 mg/kg IV did not yield measurable classical complement inhibition in the bloodstream compared with a saline control. This result is in contrast to RLS-0071 at 10 mg/kg IV which yielded a 50% decrease in C5a generation in the tissues.

These animal data demonstrate the surprising finding that RLS-0071 can inhibit complement activation in tissue, as well as inflammatory tissue damage, in multiple animal models at low doses (e.g., 10 mg/kg IV), that do not inhibit complement activation in the bloodstream.

### Example 8: RLS-0071 and Treatment of Severe Asthma

Neutrophilic asthma is a severe form of asthma which can be refractory to high doses of inhaled corticosteroids and β2-agonists, leading to frequent exacerbations and hospitalization. Currently there are no FDA-approved therapies for steroid-resistant asthma. The inventors recently adapted a neutrophilic asthma Wistar rat model mediated by intraperitoneal ovalbumin (OVA) sensitization at day 0 and 7 followed by intranasal OVA challenge at days 14 and 15 and intranasal OVA/LPS (lipopolysaccharide) challenge on days 21-23 with euthanasia of the animals at day 24. This regimen mimics the disease process observed in neutrophilic asthma patients with neutrophil infiltration into the lungs, protein accumulation suggestive of pulmonary vascular permeability and increased levels of MPO as well as free DNA indicative of neutrophil activation and neutrophil extracellular trap formation (NETosis) in the bronchoalveolar lavage fluid (BALF). The objective of this study was to evaluate the role of RLS-0071 in this animal model. Adolescent male Wistar rats subjected to this protocol were dosed intravenously with 160 mg/kg RLS-0071 on days 21-23 (prophylactic dosing) or days 22 and 23 (rescue dosing). Compared to animals not receiving RLS-0071, the BALF of animals treated with RLS-0071 showed a reduction in neutrophil count and protein levels as well as MPO and free DNA in the BALF. These results demonstrate that RLS-0071 can modulate neutrophil-mediated asthma in this rat model.

### Materials and Methods

### Animal Experiments

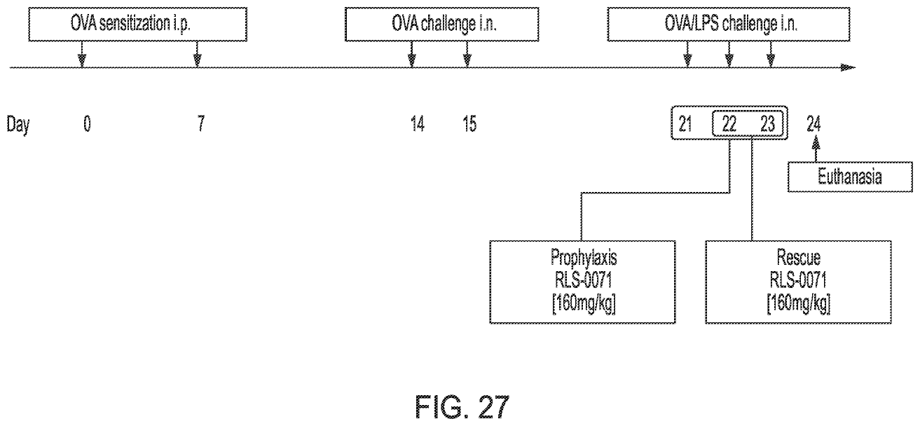

The OVA/LPS rat model of neutrophilic asthma was adapted from previously published rodent models [An T J, Rhee C K, Kim J H, Lee Y R, Chon J Y, Park C K, et al (2018) Effects of Macrolide and Corticosteroid in Neutrophilic Asthma Mouse Model. Tuberc Respir Dis (Seoul). January; 81(1):80-87. doi: 10.4046/trd.2017.0108; Thakur V R, Khuman V, Beladiya J V, Chaudagar K K, Mehta A A (2019) An experimental model of asthma in rats using ovalbumin and lipopolysaccharide allergens. Heliyon. November 19; 5(11):e02864. doi: 10.1016/j.heliyon.2019.e02864]. The experimental design is shown in FIG. 27.

For OVA (MilliporeSigma, Burlington, MA, USA) administration on Day 0 and Day 7, rats were sedated with 5% isoflurane (MWI Animal Health, Boise, ID, USA) and 1.83 mg/kg of OVA in 2 mg Al(OH)₃ solution intraperitoneal (IP) administered. For intranasal (IN) administration of OVA (0.92 mg/kg) on Days 14 and 15, rats were sedated with 5% isoflurane followed by IP administration of ketamine (McKesson, Las Colinas, TX, USA) at 75 mg/kg and xylazine (Lloyd Laboratories, Shenandoah, IA, USA) at 7 mg/kg. On Days 21, 22 and 23, rats were sedated with isoflurane and ketamine/xylazine and 0.92 mg/kg OVA and 0.18 mg/kg lipopolysaccharide (LPS, from Escherichia coli O111:B4 [MilliporeSigma, Burlington, MA, USA], reconstituted in saline and diluted into the OVA Al(OH)₃ solution) were administered IN. Animals were euthanized as described above on Day 24. For experimental groups receiving RLS-0071 treatment, the peptide was manufactured by PolyPeptide Group (San Diego, CA) to ≥95% purity as verified by HPLC and mass spectrometry analysis. Lyophilized RLS-0071 was solubilized in 0.05 M histidine buffer and pH adjusted to 6.5. RLS-0071 was administered IV through the indwelling jugular catheter at 160 mg/kg to isoflurane sedated animals on Days 21, 22 and 23 (prophylactic dosing) or Days 22 and 23 (rescue dosing) 4 hours after OVA/LPS challenge (FIG. 1). Vehicle control animals received saline without peptide IV. Animals receiving RLS-0071 and vehicle controls were sacrificed on Day 24.

Bronchoalveolar lavage fluid (BALF) was collected after euthanasia. The trachea was exposed via a midline incision, followed by the insertion of a 22-gauge 0.5-inch Luer stub (Instech Laboratories, Plymouth Meeting, PA, USA) through the tracheal rings. 1 mL of sterile saline was introduced into the lungs using a 1 mL syringe and recovered after gently massaging the chest of the rat. This was repeated 5 times for a total volume of 5 mL sterile saline. The recovered lavage fluid (approximately 4 mL) was centrifuged at 1,500 rpm for 5 min at 4° C. to pellet the cells. The BALF supernatant was collected, aliquoted, and stored at −20° C. until further analysis. The cells were resuspended in 2 mL of RPMI 1640 Medium (Thermo Fisher Scientific, Waltham, MA, USA), then cell concentrations were determined with an automated cell counter (Countess Automated Cell Counter, Thermo Fisher Scientific, Waltham, MA, USA) after staining cells with Trypan Blue dye (Thermo Fisher Scientific, Waltham, MA, USA). Cells were cytospun onto slides at a final concentration of 100,000 cells/slide for further analysis.

### Leucocyte Quantification in the BALF

The number of leukocytes present in the BALF was determined by staining cells on cytospun slides with Romanowsky-Geisma stain (Dade Behring, Deerfield, IL, USA), and slides were then thoroughly rinsed with tap water. Cells were visualized with a microscope (BX50, Olympus) at 40× magnification and different types of leukocytes (i.e., neutrophils, eosinophils, lymphocytes, and macrophages) were counted in random fields of view throughout the slide until a total of 600 cells was reached. The relative percentage of each leukocyte type was then determined. To reduce any bias during counting, the investigator was blinded, and the experimental groups were randomized.

### Protein Measurements in the BALF

The total protein concentration in the BALF supernatant was measured using the BCA Protein Assay (Thermo Fisher Scientific, Waltham, MA, USA). Briefly, 25 μL of diluted samples was mixed with 200 μL of a working reagent solution in a 96-well plate. Samples were incubated for 30 minutes at 37° C., allowed to cool for 8 minutes, then the absorbance was read at 562 nm with a BioTek microplate reader. All samples were assayed in duplicate, and the protein concentration of each sample was determined from a standard curve of known concentrations of bovine serum albumin (BSA).

### MPO Measurements in the BALF

MPO levels were measured in the BALF supernatant with a colorimetric assay. Briefly, 100 μL of sample was added in duplicate to a multi-well plate, followed by 50 μL of TMB (Thermo Fisher Scientific, Waltham, MA, USA). The reaction was incubated for 3 minutes at room temperature, then stopped with 50 μL of 2N sulfuric acid. The absorbance was read at 450 nm with a BioTek microplate reader. Known concentrations of MPO was used to generate a standard curve, which was used to calculate MPO levels in the samples.

### DNA Measurements in the BALF

Free DNA in the BALF supernatant was measured by PicoGreen. Briefly, BALF samples were diluted in 10 mM Tris-HCl, 1 mM EDTA, pH 8.0 (TE) buffer and 50 uL of each sample was added to the wells along with 50 uL of a 1:200 dilution of PicoGreen (Life Technologies, Carlsbad, CA, USA) and incubated at room temperature for 10 minutes, protected from light. A DNA standard curve was prepared in TE buffer. The fluorescence was then read at an excitation wavelength of 485 nm and an emission wavelength of 520 nm using a BioTek microplate reader. All free DNA measurements were done in duplicate.

### Statistical Analysis

Data are represented as mean and standard error of the mean. Statistical analysis was performed on the data using a Student t-test to compare significance between experimental groups. All statistical tests were performed using GraphPad Prism (San Diego, CA). All tests were two-sided with the significance level set at 0.05.

### Results

### RLS-0071 Reduces Neutrophil Levels in the BALF

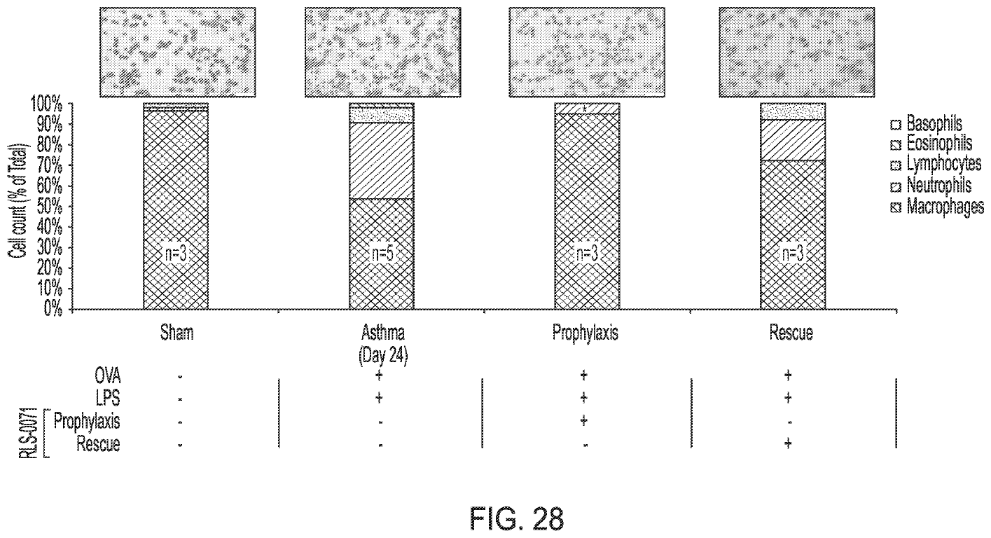

RLS-0071 is a dual targeting anti-inflammatory molecule that can inhibit both classical complement pathway activation and neutrophil effector functions (MPO activity and NETosis). To evaluate the ability of RLS-0071 to mitigate neutrophilic asthma, the inventors adapted existing murine models of neutrophil asthma that utilize intraperitoneal (IP) and intranasal (IN) infusions of OVA/LPS (FIG. 27). To determine the levels of neutrophils in animals receiving the OVA/LPS, rats were sacrificed on Day 24, the BALF was collected and leukocytes quantified by microscopy. Sham animals showed >95% alveolar macrophages in the BALF as expected (FIG. 28). In contrast, animals receiving the OVA/LPS regimen had >40% neutrophils and a >5% increase of lymphocytes in the BALF. To determine if RLS-0071 modulates neutrophil sequestration to the lungs in this model, RLS-0071 peptide was administered as a bolus dose of 160 mg/kg IV on Days 21, 22 and 23 to mimic a prophylactic dosing regimen or on Days 22 and 23 to simulate a rescue dosing scenario. Both dosing regimens were based upon peak neutrophil accumulation at Day 22 as determined in pilot experiments. Prophylactic dosing of RLS-0071 demonstrated a significant reduction in neutrophil accumulation in the BALF compared to animals receiving no peptide (P<0.03). Rescue dosing also showed a reduction in neutrophils but did not reach statistical significance (P<0.1844). These results demonstrate that IV administration of RLS-0071 can reduce neutrophil accumulation in the lungs of rats subject to neutrophilic asthma in a prophylactic or rescue dosing scenario.

### RLS-0071 Reduces Protein Levels in the BALF

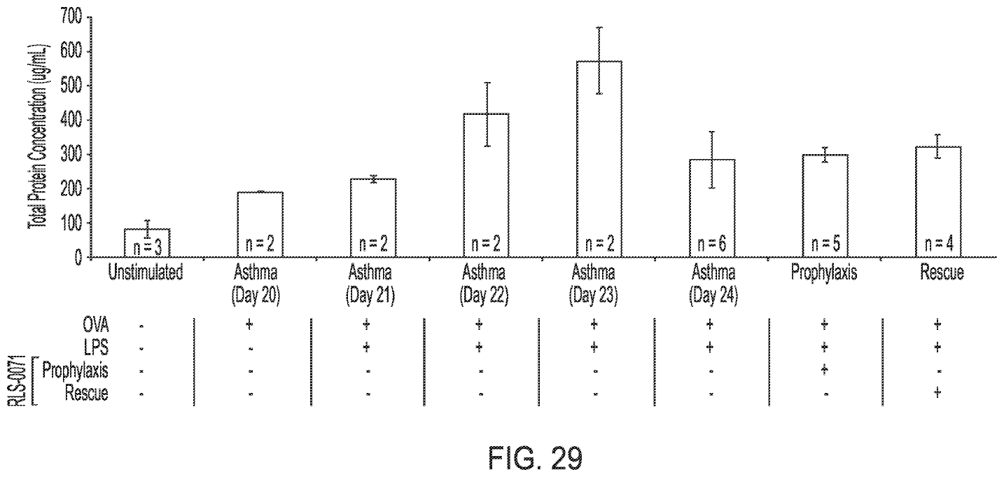

To ascertain the level of pulmonary vascular leakage in animals receiving the OVA/LPS regimen, animals were sacrificed at Days 20-24, the BALF was collected and total protein concentration determined. Compared with sham rats, animals receiving the OVA/LPS protocol showed increasing levels of protein in the BALF on Days 20-23 with drop off on Day 24 most likely indicative of protein reabsorption into the vascular tissue (FIG. 29). Consistent with these findings, animals receiving prophylactical or rescue dosing of RLS-0071 had similar levels of protein as asthma rats on Day 24.

### RLS-0071 Reduces MPO Levels and Free DNA in the BALF

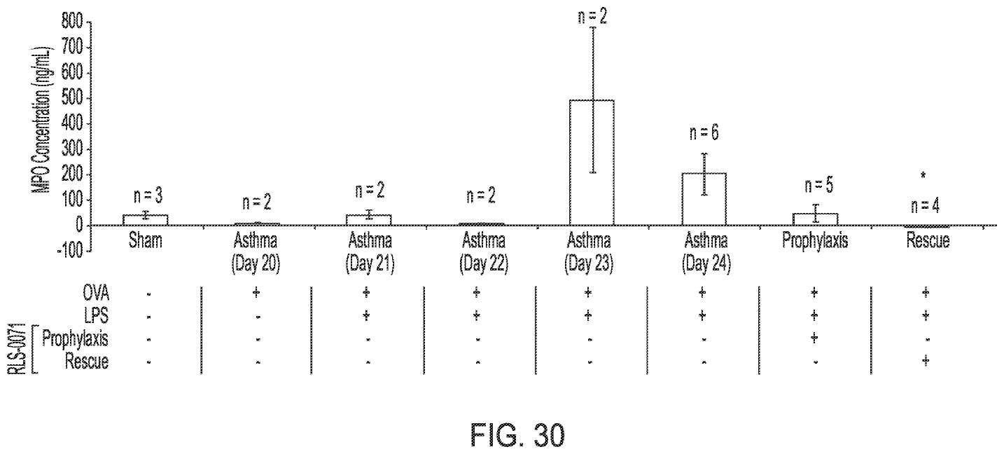

To ascertain the effect of RLS-0071 on MPO levels in the BALF of animals receiving the OVA/LPS protocol, animals were sacrificed at Days 20-24, the BALF was collected and total MPO concentration determined. Sham rats and animals receiving the OVA/LPS regimen showed background levels of free MPO when the BALF was collected on Days 20-22 (FIG. 30). MPO levels increased dramatically on Day 23 and tapered down by Day 24 in asthma rats. Animals receiving RLS-0071 as a prophylactic regimen showed a reduction in MPO levels that did not reach statistical significance (p=0.12) compared to Day 24 animals that did not receive peptide, whereas rescue dosing showed a significant reduction in MPO levels (P=0.05).

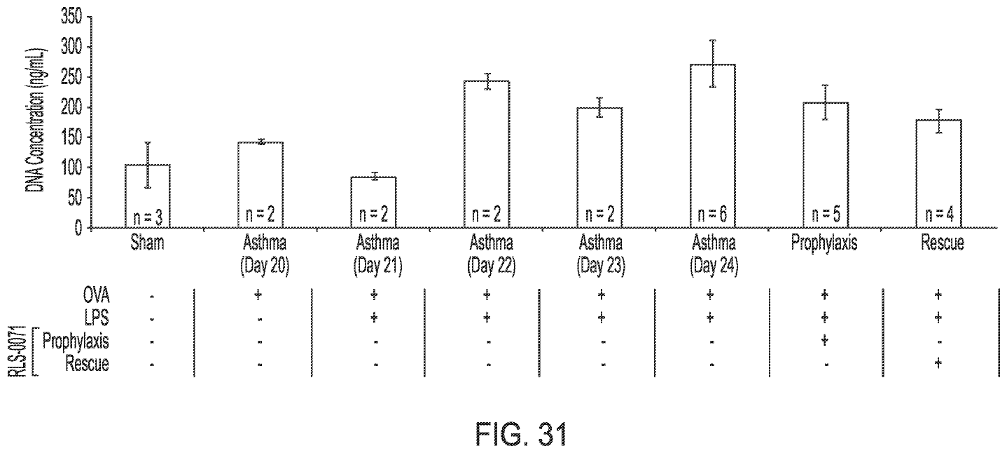

MPO is a key player in production of neutrophil extracellular traps (NETs). It combines with hydrogen peroxide in neutrophil granules to mediate NETosis and RLS-0071 has been shown to inhibit the formation of NETs in vitro. NETs have been previously shown to play a pathogenic role in a variety of autoimmune, metabolic, and inflammatory diseases including neutrophilic asthma [Lachowicz-Scroggins M E, Dunican E M, Charbit A R, Raymond W, Looney M R, Peters M C, et al. (2019) Extracellular DNA, Neutrophil Extracellular Traps, and Inflammasome Activation in Severe Asthma. Am J Respir Crit Care Med. 199(9):1076-1085; Varricchi G, Modestino L, Poto R, Cristinziano L, Gentile L, Postiglione L, et al. (2021) Neutrophil extracellular traps and neutrophil-derived mediators as possible biomarkers in bronchial asthma. Clin Exp Med. 2021 Aug. 3. doi: 10.1007/s10238-021-00750-8]. To ascertain the effect on NET formation in the OVA/LPS treated animals, free DNA from the BALF was determined. Free or extracelluar DNA is frequently used as a biomarker for NET formation in autoimmune and inflammatory diseases. Low levels of free DNA were observed in the BALF from sham animals and asthma animals isolated on Days 20 and 21 with an increase in free DNA in the BALF harvested from asthma animals on Days 22, 23, and 24 (FIG. 31). In animals dosed with RLS-0071 prophylactically or in a rescue dosing regimen, a decrease in free DNA was observed compared to free DNA levels from asthma rats on Days 22-24, however the levels of free DNA did not return to baseline levels as seen in sham animals. Without wishing to be bound by theory, it is possible that the reduction in MPO and free DNA levels demonstrates that RLS-0071 can reduce neutrophil mediated effector functions in the BALF of animals subject to neutrophilic asthma.

### Discussion

The objective of this Example was to determine if the anti-inflammatory molecule RLS-0071 was able to mitigate severe or neutrophilic asthma in an OVA/LPS murine model adapted from the literature [An T J, Rhee C K, Kim J H, Lee Y R, Chon J Y, Park C K, et al (2018) Effects of Macrolide and Corticosteroid in Neutrophilic Asthma Mouse Model. Tuberc Respir Dis (Seoul). January; 81(1):80-87. doi: 10.4046/trd.2017.0108; Thakur V R, Khuman V, Beladiya J V, Chaudagar K K, Mehta A A (2019) An experimental model of asthma in rats using ovalbumin and lipopolysaccharide allergens. Heliyon. November 19; 5(11):e02864. doi: 10.1016/j.heliyon.2019.e02864]. As noted by others, the OVA/LPS regimen resulted in neutrophil influx into the lungs, vascular inflammation, and neutrophil activation as evidenced by released MPO and free DNA indicative of NET formation. RLS-0071 has been demonstrated to inhibit classical complement activation in in vitro, in vivo and ex vivo studies and inhibit NET formation via inhibition of MPO in in vitro and ex vivo studies. Given the dual anti-inflammatory activities of complement inhibition and neutrophil modulation, the inventors hypothesized that RLS-0071 would inhibit neutrophilic asthma in this animal model. The results presented herein demonstrate that RLS-0071 delivered either prophylactically or as a rescue dose was able to reduce neutrophil sequestration and activation in the lung. This was demonstrated by reduced neutrophil counts in the lung, and decreased levels of protein, MPO and free DNA which serves as a biomarker for NETosis in the BALF.

Asthma is a chronic, complicated, inflammatory disease with a variety of inflammatory cells (eosinophils, basophils, neutrophils, monocytes, macrophages and activated mast cells) playing a pathological role. A number of inflammatory mediators such as interleukins, cytokines and leukotrienes released from inflammatory cells contribute to the inflammation characteristic of asthma and it is believed that the activation of type 1 helper T cell (Th1) and type 2 helper T cell (Th2) by allergens play a prominent role [P. J. Barnes (1996) Pathophysiology of asthma, Br. J. Clin. Pharmacol. 42 (1) 3-10]. Animal models using the dual allergen challenge of OVA and LPS have demonstrated a Th1 helper T cell response, mediated presumably mediated by LPS activation of TLR-4 leads to a severe form of asthma driven by neutrophilic activation. This neutrophil-driven disease process mimics severe asthma seen in humans which is refractory to steroid or b2-agonists. Without wishing to be bound by theory, it is suggested that the ability of RLS-0071 to mitigate neutrophilic asthma in this rodent model indicates that RLS-0071 has potential for utility as a clinical therapeutic for steroid refractory, neutrophilic asthma. Additionally, it may have efficacy in other acute neutrophil-mediated pulmonary exacerbations characterized by a dysregulated immune response, such as COPD.

### Example 9: RLS-0071 and RLS-0088-Mediated Modulation of Angiogenesis and Binding to VEGF

### RLS-Peptides Binding to VEGF and Inhibition of VEGF Signaling in a Cell-Based Bioassay

Vascular endothelial growth factor (VEGF) is an important signaling protein that is secreted from epithelial cells, tumor cells and macrophages. It has many functions, including stimulation of angiogenesis, increase of vascular permeability, enhancement of tumor invasion and survival, and inhibition of antitumor response in Treg cells. There are several VEGF receptor subtypes-VEGFR1, VEGFR2 and VEGFR3. VEGFR2 (also known as KDR) mediates almost all of the known receptor cellular responses to VEGF. All members of the VEGF family stimulate cellular response by binding to receptors of the receptor tyrosine kinase, namely VEGFR-1 (Flt-1) and VEGFR-2 (Flk-1/KDR). When VEGF binds to KDR, the receptor dimerizes and becomes activated through transphosphorylation.

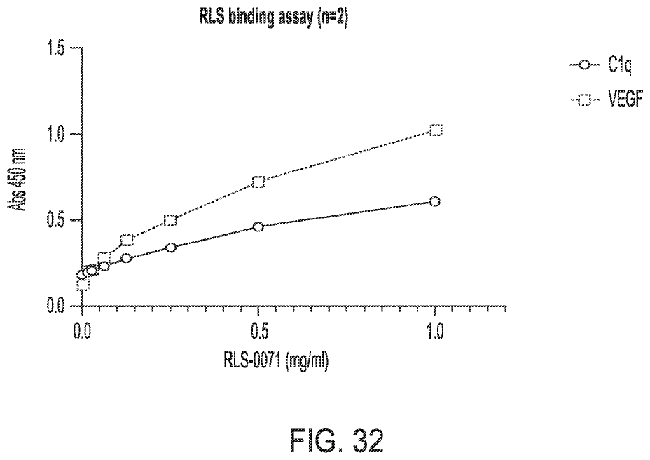

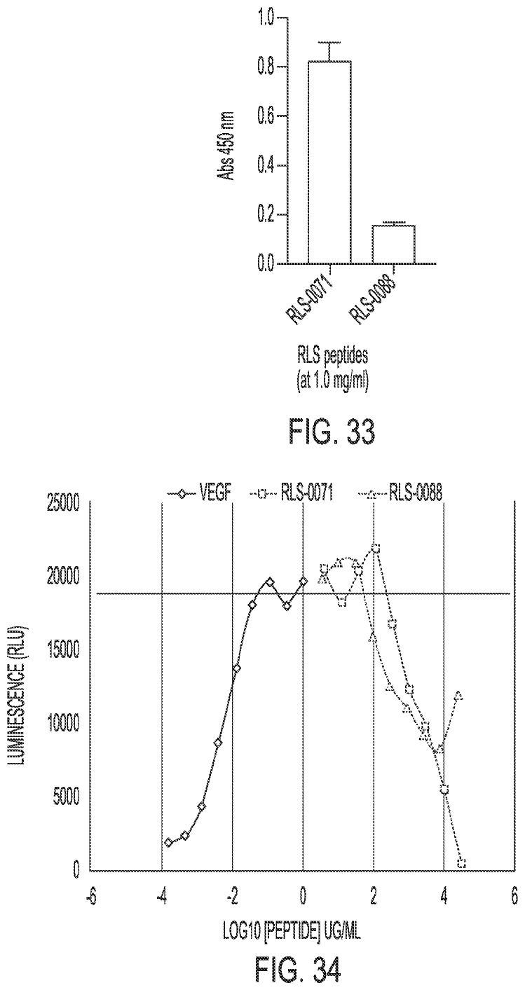

RLS-0071 is shown herein to downregulate VEGF in the inventors' 2-hit rat acute lung injury model (FIG. 4). The inventors wished to determine if RLS-0071 and RLS-0088 could directly interact with human VEGF in an ELISA based assay. VEGF was coated onto a microtiter plate and incubated with RLS-0071 at increasing concentration which were subsequently detected with an antibody to the peptide, followed by secondary antibody-HRP conjugate. The signal generated from the HRP conjugate was then read in a plate reader at an OD of 450 nm. As shown in FIG. 32, RLS-0071 dose-dependently binds human VEGF to a greater degree than C1q (positive control). While there is significant binding of RLS-0071 to VEGF, binding of RLS-0088 was much less pronounced (FIG. 33). To determine if binding of VEGF correlated with functional activity, the inventors utilized a VEGF bioassay (Promega), which is a bioluminescent cell-based assay that measures VEGF stimulation and inhibition of KDR (VEGFR-2) using luciferase as a readout. This assay is used for discovery and development of novel biologic therapies aimed at either inducing or inhibiting the VEGF response. The VEGF responsive cells have been engineered to express the response element (RE) upstream of luc2P, as well as exogenous VEGF receptor. When VEGF binds to VEGF responsive cells, the receptor transduces intracellular signals resulting in luminescence. The bioluminescent signal is detected and quantified using Bio-Glo™ Luciferase Assay System and a standard luminometer. In this assay, VEGF was a positive control, and increasing concentrations of VEGF result in a dose-dependent increase in luminescence, indicative of VEGF binding to VEGFR-2 and affecting intracellular signaling (FIG. 34, line marked with diamonds). RLS-0071 and RLS-0088 were both able to inhibit VEGF binding to VEGFR-2 resulting in a dose-dependent inhibition of intracellular signaling (FIG. 34, lines marked with squares and triangles, respectively). These results demonstrate the surprising finding that RLS-0071 and RLS-0088 can inhibit VEGF-mediated signaling. Without wishing to be bound by theory, it is suggested that RLS-0071 and RLS-0088 may have utility as therapeutic molecules to inhibit various VEGF-mediated disease processes.

### RLS-Peptides Inhibition of Non-VEGF Mediated Angiogenesis in a Cell-Based Assay

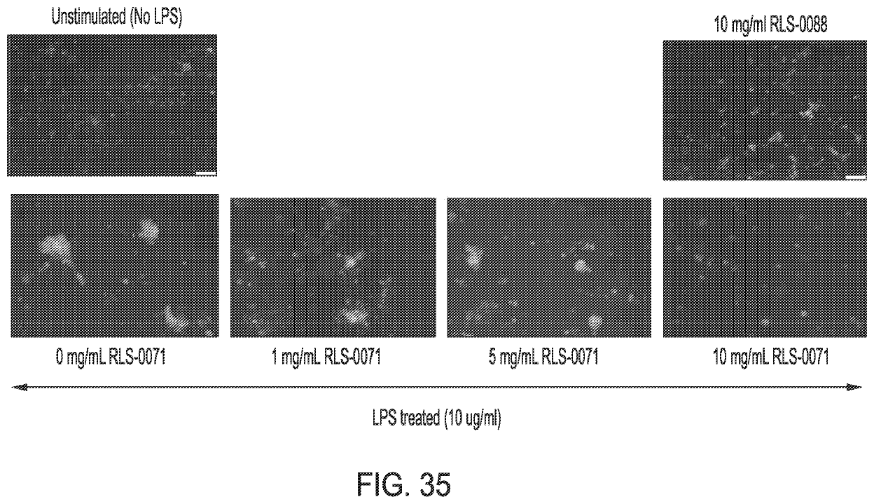

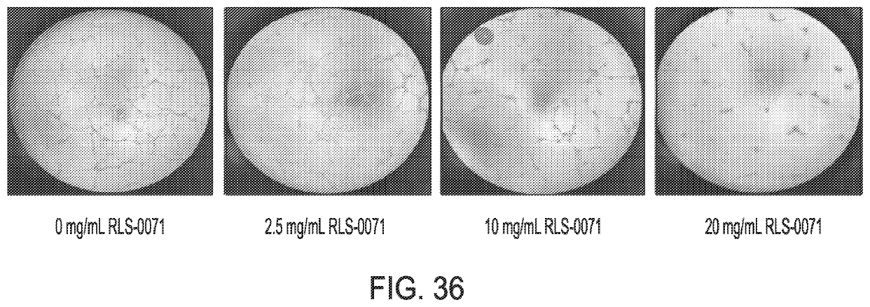

To ascertain if RLS-0071 and RLS-0088 can inhibit angiogenesis in a VEGF-independent manner, the inventors utilized a human umbilical vascular endothelial cell (HUVEC) 3-dimensional culture system. In this system, HUVEC cells were stained with CellTrace dye, pre-treated with the peptides for 1 hour at 37° C., mixed with an extracellular matrix (Sigma) that contains 10 ug/ml of lipopolysaccharide (LPS), plated, and then incubated in a humidified incubator at 37° C. for 18 hours. LPS can cause the cells to undergo non-VEGF mediated angiogenesis, which results in the formation of endothelial sprouting and tube formation that can be observed by microscopy. As shown in FIGS. 35 and 36, cells not receiving LPS (unstimulated (No LPS)) had no observable sign of angiogenesis, whereas cells that were treated with LPS and no peptide (0 mg/ml RLS-0071 panel) show sprouting and nascent tube formation indicative of angiogenesis. In the presence of increasing amounts of RLS-0071, a dose-dependent reduction in angiogenesis was observed. RLS-0088 also demonstrated a reduction in angiogenesis at a concentration of 10 mg/ml of peptide. The same results were obtained with RLS-0071 in a different HUVEC cell system that used a different extracellular matrix (Geltrex, Sigma) and no CellTrace dye. See also Table 3, showing the relative activity of RLS-0071 and RLS-0088 in each of these assays. These results demonstrate the surprising finding that RLS-0071 and RLS-0088 can inhibit non-VEGF mediated angiogenesis. Without wishing to be bound by theory, it is possible that RLS-0071 and RLS-0088 can have potential as anti-angiogenic therapeutic molecules.

TABLE 3
Relative activity of RLS-0071 and RLS-0088

| Peptide | VEGF | VEGF Bioassay | Anti- |
|---|---|---|---|
| Code | Binding | inhibition | angiogenesis |
| RLS-0071 | +++ | ++ | ++++ |
| RLS-0088 | +/− | +++ | ++ |

### Example 10: Administration of Ophthalmic Formulations

An ophthalmic composition comprising a therapeutically effective amount of SEQ ID NO: 2 is administered to a subject's eye to treat an ophthalmic disease or condition. The administration can be topical (e.g., ointment, eye drops, foam, eye packs), via injection (e.g., intra-vitreal injection, intra-aqueous injection, subconjunctival injection), or by via implantation of an intraocular or intravitreal implant. The ophthalmic disease or condition may be characterized by an altered expression of a cell surface receptor, such as an integrin or an ICAM, e.g., ICAM-1, ICAM-3, ICAM-4, and/or ICAM-5. Non-limiting exemplary ophthalmic diseases or conditions include autoimmune and infectious uveitis, retinitis, AMD, DED, infectious and non-infectious keratitis, corneal injury and repair, retinopathy of prematurity (ROP), ocular graft versus host disease (GvHD), diabetic retinopathy, macular edema following retinal vein occlusion (RVO) and diabetic macular edema (DME).

### Example 11: Administration of Nasal Formulations

A nasal composition comprising a therapeutically effective amount of SEQ ID NO: 2 is administered to a subject to treat asthma. The administration can be via inhalation, insufflation, or nebulization. The composition can be in the form of a spray, solution, gel, cream, lotion, aerosol or solution for a nebulizer, or as a microfine powder for insufflation. The asthma may be characterized by an altered expression of a cell surface receptor, such as an integrin or an ICAM, e.g., ICAM-1, ICAM-3, ICAM-4, and/or ICAM-5. Non-limiting exemplary types of asthma include severe asthma, steroid-refractory asthma, and neutrophilic asthma.

### Example 11: Administration of Pharmaceutical Formulations

A pharmaceutical composition comprising a therapeutically effective amount of SEQ ID NO: 2 and/or 3 is administered to a subject in need thereof to treat a disease or condition. The administration can be by any appropriate route (e.g., injection, infusion, implantation, topical administration, nasal administration). The disease or condition may be characterized by an altered expression of a cell surface receptor such as an integrin or an ICAM, e.g., ICAM1, ICAM-3, ICAM-4, and/or ICAM-5.

A pharmaceutical composition comprising a therapeutically effective amount of SEQ ID NO: 2 and/or 3 is administered to a subject in need thereof to regulate the complement system in the subject. The administration can be by any appropriate route (e.g., injection, infusion, implantation, topical administration, nasal administration).

A pharmaceutical composition comprising a therapeutically effective amount of SEQ ID NO: 2 and/or 3 is administered to a subject in need thereof to alter cytokine expression in the subject. The administration can be by any appropriate route (e.g., injection, infusion, implantation, topical administration, nasal administration).

A pharmaceutical composition comprising a therapeutically effective amount of SEQ ID NO: 2 and/or 3 is administered to a subject in need thereof to inhibit or alter neutrophil binding and/or adhesion in the subject. The administration can be by any appropriate route (e.g., injection, infusion, implantation, topical administration, nasal administration).

A pharmaceutical composition comprising a therapeutically effective amount of SEQ ID NO: 2 and/or 3 is administered to a subject in need thereof to improve neutrophil survival in the subject. The administration can be by any appropriate route (e.g., injection, infusion, implantation, topical administration, nasal administration).

A pharmaceutical composition comprising a therapeutically effective amount of SEQ ID NO: 2 and/or 3 is administered to a subject in need thereof to inhibit or alter neutrophil binding to cell surface receptors in the subject. The administration can be by any appropriate route (e.g., injection, infusion, implantation, topical administration, nasal administration). Non-limiting examples of cell surface receptors include integrins and ICAMs, e.g., ICAM-1, ICAM-3, ICAM-4, and ICAM-5.

The following is a non-exhaustive list of items encompassed in the present invention.
1. A method of altering cytokine expression comprising administering to the subject in need thereof a composition comprising a therapeutically effective amount of a synthetic peptide comprising SEQ ID NO: 2 and/or 3.
2. A method of inhibiting or altering neutrophil binding and/or adhesion comprising administering to the subject in need thereof a composition comprising a therapeutically effective amount of a synthetic peptide comprising SEQ ID NO: 2 and/or 3.
3. A method of improving neutrophil survival comprising administering to the subject in need thereof a composition comprising a therapeutically effective amount of a synthetic peptide comprising SEQ ID NO: 2 and/or 3.
4. A method of inhibiting or altering neutrophil binding to cell surface receptors comprising administering to the subject in need thereof a composition comprising a therapeutically effective amount of a synthetic peptide comprising SEQ ID NO: 2 and/or 3.
5. A method of treating a disease or condition characterized by an altered expression of a cell surface receptor comprising administering a composition comprising a therapeutically effective amount of a synthetic peptide comprising SEQ ID NO: 2 and/or 3.
6. A method of treating and/or preventing acute lung injury and/or acute respiratory distress syndrome comprising administering a composition comprising a therapeutically effective amount of a synthetic peptide comprising SEQ ID NO: 2 and/or 3.
7. A method of treating and/or preventing an ocular disease and/or condition characterized by dysregulated complement activation and/or neutrophil modulation comprising administering a composition comprising a therapeutically effective amount of a synthetic peptide comprising SEQ ID NO: 2.
8. A method of treating asthma comprising administering a composition comprising a therapeutically effective amount of a synthetic peptide comprising SEQ ID NO: 2.
9. A method of modulating angiogenesis comprising administering a composition comprising a therapeutically effective amount of a synthetic peptide comprising SEQ ID NO: 2 and/or 3.
10. The methods of any of items 1-9, wherein the composition further comprises at least one pharmaceutically acceptable carrier, diluent, stabilizer, or excipient.
11. The methods of any of items 1-10, wherein the therapeutically effective amount of SEQ ID NO: 2 and/or 3 is about 10 mg/kg to about 160 mg/kg.
12. The methods of any of items 1-10, wherein the therapeutically effective amount of SEQ ID NO: 2 and/or 3 is about 20 mg/kg to about 160 mg/kg.
13. The methods of any of items 1-10, wherein the therapeutically effective amount of SEQ ID NO: 2 and/or 3 is about 40 mg/kg to about 160 mg/kg.
14. The methods of any of items 1-10, wherein the therapeutically effective amount of SEQ ID NO: 2 and/or 3 is administered in at least one dose, the first dose comprising about 10 mg/kg to about 160 mg/kg SEQ ID NO: 2 and/or 3.
15. The method of item 14, wherein a second dose comprising a therapeutically effective amount of SEQ ID NO: 2 and/or 3 is administered, the second dose comprising about 40 mg/kg to about 60 mg/kg SEQ ID NO: 2 and/or 3.
16. The method of any of items 1-10, wherein the therapeutically effective amount of SEQ ID NO: 2 and/or 3 is administered in two doses, the first dose comprising about 10 mg/kg to about 160 mg/kg SEQ ID NO: 2 and/or 3 and the second dose comprising about 40 mg/kg to about 60 mg/kg SEQ ID NO: 2 and/or 3.
17. The methods of any of items 1-16, wherein the composition is formulated for ophthalmic administration.
18. The method of item 17, wherein the composition further comprises an ophthalmically acceptable carrier and/or excipient.
19. The method of items 17 or 18, wherein the ophthalmic administration comprises topical administration, periocular injection, subconjunctival injection, intra-aqueous injection, intraocular injection, intravitreal injection, or introduction of an intracorneal or intraocular implant.
19. The method of item 4, wherein the cell surface receptor comprises an integrin or an intercellular adhesion molecule (ICAM).
20. The method of item 19, wherein the ICAM comprises ICAM-1, ICAM-3, ICAM-4, and/or ICAM-5.
21. The method of item 5, wherein the disease or condition is characterized by an increase in at least one of ICAM-1, ICAM-3, ICAM-4, and/or ICAM-5.
22. The method of item 7, wherein the ocular disease or condition is characterized by complement inhibition and/or inhibition of myeloperoxidase activity or NETosis.
23. The method of item 7, wherein the ocular disease or condition is autoimmune and infectious uveitis, acute macular degeneration (AMD), dry eye disease (DED), infectious and non-infectious keratitis, corneal injury and repair, retinopathy of prematurity (ROP), ocular graft versus host disease (GvHD), diabetic retinopathy, macular edema following retinal vein occlusion (RVO) and diabetic macular edema (DME).
24. The method of item 8, wherein the asthma is severe asthma, steroid-refractory asthma, or neutrophilic asthma.
25. The method of any of items 1-16, wherein the composition is formulated for nasal administration.
26. The method of item 25, wherein the nasal administration comprises inhalation, insufflation, or nebulization.
27. The method of item 25, wherein the composition is in the form of a spray, solution, gel, cream, lotion, aerosol or solution for a nebulizer, or as a microfine powder for insufflation.

### SEQUENCE LISTING

SEQ ID NO: 1:
IALILEPICCQERAA
SEQ ID NO: 2:
IALILEPICCQERAA-dPEG24,
containing a C-terminal monodisperse
24-mer PEGylated moiety (RLS-0071;
PA-dPEG24; SEQ ID NO: 2)
SEQ ID NO: 3:
IALILEP(Sar)CCQERAA,
containing a sarcosine residue
at position 8 (RLS-0088;
PA-I8Sar; SEQ ID NO: 3)

While several possible embodiments are disclosed above, embodiments of the present invention are not so limited. These exemplary embodiments are not intended to be exhaustive or to unnecessarily limit the scope of the invention, but instead were chosen and described in order to explain the principles of the present invention so that others skilled in the art may practice the invention. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

1. Fosgerau K and Hoffmann T (2014) Peptide therapeutics: current status and future directions. Drug Disc Today 20: 122-129.
2. Ali A M, Atmaj J, Van Oosterwijk N, Groves M R, Domling A (2019) Stapled peptide inhibitors: a new window for target drug discovery. Comp Struct Biotech J 17: 263-281
3. Sharp J A, Hair P S, Pallera H K, Kumar P S, Mauriello C T, et al. (2015) Peptide Inhibitor of Complement C1 (PIC1) Rapidly Inhibits Complement Activation after Intravascular Injection in Rats. PLoS ONE 10: e0132446.
4. Hair P S, Sass L A, Krishna N K, Cunnion K M (2017) Inhibition of Myeloperoxidase Activity in Cystic Fibrosis Sputum by Peptide Inhibitor of Complement C1 (PIC1). PLoS ONE 12: e0170203.
5. Hair P S, Cunnion K M, Krishna N K (2017) Peptide Inhibitor of Complement C1 Inhibits the Peroxidase Activity of Hemoglobin and Myoglobin. Int J Pept 2017: 9454583.
6. Gregory Rivera M, Hair P S, Cunnion K M, Krishna N K (2018) Peptide Inhibitor of Complement C1 (PIC1) demonstrates antioxidant activity via single electron transport (SET) and hydrogen atom transfer (HAT). PLoS ONE 13: e0193931.
7. Hair P S, Enos A I, Krishna N K, Cunnion K M (2018) Inhibition of Immune Complex Complement Activation and Neutrophil Extracellular Trap Formation by Peptide Inhibitor of Complement C1. Front Immunol 9: 558.
8. Hair P S, Rivera M G, Enos A I, Pearsall S E, Sharp J A, et al. (2017) Peptide Inhibitor of Complement C1 (PIC1) Inhibits Growth of Pathogenic Bacteria. International Journal of Peptide Research and Therapeutics DOI 101007/s10989-017-9651-z.
9. Matsui S M, Kiang D, Ginzton N, Chew T, Geigenmuller-Gnirke U (2001) Molecular biology of astroviruses: selected highlights. Novartis Found Symp 238: 219-233; discussion 233-216.
10. Bonaparte R S, Hair P S, Banthia D, Marshall D M, Cunnion K M, et al. (2008) Human astrovirus coat protein inhibits serum complement activation via C1, the first component of the classical pathway. J Virol 82: 817-827.
11. Hair P S, Enos A I, Krishna N K, Cunnion K M. (2019) Inhibition of complement activation, myeloperoxidase, NET formation and oxidant activity by PIC1 peptide variants. PLoS ONE 14: e0226875.
12. Cunnion K M, Lee J C, Frank M M (2001) Capsule production and growth phase influence binding of complement to Staphylococcus aureus. Infect Immun 69: 6796-6803.
Barnes B J, Adrover J M, Baxter-Stoltzfus A, et al. Targeting potential drivers of COVID-19: Neutrophil extracellular traps. J Exp Med. 2020 Jun. 1; 217(6).
Caudrillier A, Kessenbrock K, Gilliss B M, et al. Platelets induce neutrophil extracellular traps in transfusion-related acute lung injury. J Clin Invest. 2012 July; 122(7):2661-71.
Hair P S, Enos A I, Krishna N K, et al. Inhibition of Immune Complex Complement Activation and Neutrophil Extracellular Trap Formation by Peptide Inhibitor of Complement C1. Front Immunol. 2018; 9:558.
Mehta P, McAuley D F, Brown M, et al. COVID-19: consider cytokine storm syndromes and immunosuppression. Lancet. 2020 Mar. 28; 395(10229):1033-1034.
Rivera G M, Sampson A C, Hair P S, et al. Incompatible erythrocyte transfusion with lipopolysaccharide induces acute lung injury in a novel rat model. PLoS One. 2020; 1-25 in press.
Silliman C C, Paterson A J, Dickey W O, et al. The association of biologically active lipids with the development of transfusion-related acute lung injury: a retrospective study. Transfusion. 1997 July; 37(7):719-26.
Silliman C C. The two-event model of transfusion-related acute lung injury. Crit Care Med. 2006 May; 34(5 Suppl):S124-31.
Thomas G M, Carbo C, Curtis B R, et al. Extracellular DNA traps are associated with the pathogenesis of TRALI in humans and mice. Blood. 2012 Jun. 28; 119(26):6335-43.
Jha P, Bora P S, Bora N S. The complement system and ocular diseases. Mol Immunol. 2007 September; 44(16):3901-3908.
Estua-Acosta G A, Zamora-Ortiz R, Buentello-Volante B, et al. Neutrophil extracellular traps: current perspectives in the eye. Cells. 2019; 8:979.
Ghosh S, Padmanabhan A, Vaidya T et al. Neutrophils homing into the retina trigger pathology in early age-related macular degeneration. Commun Biol. 2019; 2:348:1-17.
Cooper N R. The classical complement pathway: activation and regulation of the first complement component. Adv Immunol. 1985; 37:151-216.
Thielens N M, Tedesco F, Bohlson S S. C1q: A fresh look upon an old molecule. Mol Immunol. 2017; September; 89:73-83.
Sharp J A, Hair P S, Pallera H K, et al. Peptide Inhibitor of Complement C1 (PIC1) Rapidly Inhibits Complement Activation after Intravascular Injection in Rats. PLoS ONE. 2015; 10(7): e0132446.
Mica J, Jor O, Holub M, Sklienka P, Bursa F, Burda M, et al. Past and Present ARDS Mortality Rates: A Systematic Review. Respir Care 2017; 62(1):113-122.
Cho M S, Modi P, Sharma S. Transfusion-related Acute Lung Injury. 2020; In: StatPearls. Treasure Island (F L): StatPearls Publishing; 2020 January
Kumar A K, Anjum F. Ventilator-Induced Lung Injury (VILI). 2020 Dec. 15. In: StatPearls. Treasure Island (F L): StatPearls Publishing; 2020 January
Arroyo-Hernández M, Maldonado F, Lozano-Ruiz F, Muñoz-Montano W, Nunez-Baez M, Arrieta O. Radiation-induced lung injury: current evidence. BMC Pulm Med 2021; 21(1):9.
Klomp M, Ghosh S, Mohammed S, Nadeem Khan M. From virus to inflammation, how influenza promotes lung damage. J Leukoc Biol 2020; September 8.
Alvarez A E, Marson F A, Bertuzzo C S, Arns C W, Ribeiro J D. Epidemiological and genetic characteristics associated with the severity of acute viral bronchiolitis by respiratory syncytial virus. J Pediatr (Rio J)2013; 89(6):531-43.
Lee C, Choi W J. Overview of COVID-19 inflammatory pathogenesis from the therapeutic perspective. Arch Pharm Res 2021; January 4:1-18.
Pandya P H, Wilkes D S. Complement system in lung disease. Am J Respir Cell Mol Biol 2014; 51:467-473. doi: 10.1165/rcmb.2013-0485T R.
Khan M A, Nicolls M R, Surguladze B, Saadoun I. Complement components as potential therapeutic targets for asthma treatment. Respir Med 2014; 108:543-549. doi: 10.1016/j.rmed.2014.01.005.
Silliman C C, Paterson A J, Dickey W O, Stroneck D F, Popovsky M A, Caldwell S A, et al. The association of biologically active lipids with the development of transfusion-related acute lung injury: a retrospective study. Transfusion 1997; 37(7):719-26.
Silliman C C, McLaughlin N J. Transfusion-related acute lung injury. Blood Rev 2006; 20(3):139-59.
Rebetz J, Semple J W, Kapur R. The Pathogenic Involvement of Neutrophils in Acute Respiratory Distress Syndrome and Transfusion-Related Acute Lung Injury. Transfus Med Hemother 2018; 45(5):290-298.
Jongerius I, Porcelijn L, van Beek A E, Semple J W, van der Schoot C E, Vlaar A P J, et al. The Role of Complement in Transfusion-Related Acute Lung Injury. Transfus Med Rev 2019; 33(4):236-242.
Polidoro R B, Hagan R S, de Santis Santiago R, Schmidt N W. Overview: Systemic Inflammatory Response Derived From Lung Injury Caused by SARS-CoV-2 Infection Explains Severe Outcomes in COVID-19. Front Immunol 2020; 11:1626.
Gregory Rivera M, Hair P S, Cunnion K M, Krishna N K. Peptide Inhibitor of Complement C1 (PIC1) demonstrates antioxidant activity via single electron transport (SET) and hydrogen atom transfer (HAT). PLoS One 2018; 13(3):e0193931.

1. (canceled)

2. A method of inhibiting or altering neutrophil binding and/or adhesion comprising administering to the subject in need thereof a composition comprising a therapeutically effective amount of a synthetic peptide comprising SEQ ID NO: 2 and/or 3.

3. (canceled)

4. A method of inhibiting or altering neutrophil binding to cell surface receptors comprising administering to the subject in need thereof a composition comprising a therapeutically effective amount of a synthetic peptide comprising SEQ ID NO: 2 and/or 3.

5. A method of treating a disease or condition characterized by an altered expression of a cell surface receptor and/or dysregulated complement activation and/or neutrophil modulation, the method comprising administering a composition comprising a therapeutically effective amount of a synthetic peptide comprising SEQ ID NO: 2 and/or 3.

6-9. (canceled)

10. The method of claim 2, wherein the composition further comprises at least one pharmaceutically acceptable carrier, diluent, stabilizer, or excipient.

11. The method of claim 2, wherein the therapeutically effective amount of SEQ ID NO: 2 and/or 3 is about 10 mg/kg to about 160 mg/kg.

12. The method of claim 2, wherein the therapeutically effective amount of SEQ ID NO: 2 and/or 3 is about 20 mg/kg to about 160 mg/kg.

13. The method of claim 2, wherein the therapeutically effective amount of SEQ ID NO: 2 and/or 3 is about 40 mg/kg to about 160 mg/kg.

14. The method of claim 2, wherein the therapeutically effective amount of SEQ ID NO: 2 and/or 3 is administered in at least one dose, the first dose comprising about 10 mg/kg to about 160 mg/kg SEQ ID NO: 2 and/or 3.

15. The method of claim 14, wherein a second dose comprising a therapeutically effective amount of SEQ ID NO: 2 and/or 3 is administered, the second dose comprising about 40 mg/kg to about 60 mg/kg SEQ ID NO: 2 and/or 3.

16. The method of claim 2, wherein the therapeutically effective amount of SEQ ID NO: 2 and/or 3 is administered in two doses, the first dose comprising about 10 mg/kg to about 160 mg/kg SEQ ID NO: 2 and/or 3 and the second dose comprising about 40 mg/kg to about 60 mg/kg SEQ ID NO: 2 and/or 3.

17. The method of claim 5, wherein the composition is formulated for ophthalmic administration.

18. The method of claim 17, wherein the composition further comprises an ophthalmically acceptable carrier and/or excipient.

19. The method of claim 17, wherein the ophthalmic administration comprises topical administration, periocular injection, subconjunctival injection, intra-aqueous injection, intraocular injection, intravitreal injection, or introduction of an intracorneal or intraocular implant.

20. The method of claim 4, wherein the cell surface receptor comprises an integrin or an intercellular adhesion molecule (ICAM).

21-27. (canceled)

28. The method of claim 5, wherein the disease or condition is an ocular disease or condition characterized by complement inhibition and/or inhibition of myeloperoxidase activity or NETosis.

29. The method of claim 28, wherein the ocular disease or condition is autoimmune and infectious uveitis, acute macular degeneration (AMD), dry eye disease (DED), infectious and non-infectious keratitis, corneal injury and repair, retinopathy of prematurity (ROP), ocular graft versus host disease (GvHD), diabetic retinopathy, macular edema following retinal vein occlusion (RVO) and diabetic macular edema (DME).

30. The method of claim 5, wherein the disease or condition is severe asthma, steroid-refractory asthma, or neutrophilic asthma.

31. The method of claim 30, wherein the composition is formulated for nasal administration.

32. The method of claim 31, wherein the nasal administration comprises inhalation, insufflation, or nebulization.

33. The method of claim 31, wherein the composition is in the form of a spray, solution, gel, cream, lotion, aerosol or solution for a nebulizer, or as a microfine powder for insufflation.


SEQUENCE LISTING (3 sequences)

SEQ ID NO: 1 - protein, 15 aa, Artificial Sequence
  Description of Artificial Sequence Synthetic peptide
IALILEPICCQERAA

SEQ ID NO: 2 - protein, 15 aa, Artificial Sequence
  Description of Artificial Sequence Synthetic peptide
  C-terminal monodisperse 24-mer PEGylated moiety
IALILEPICCQERAA

SEQ ID NO: 3 - protein, 15 aa, Artificial Sequence
  Description of Artificial Sequence Synthetic peptide
  MOD_RES (8)..(8): Sarcosine
IALILEPXCCQERAA